(19) United States
(12) Patent Application Publication
(10) Pub. No.: US 2012/0282262 A1
(43) Pub. Date: Nov. 8, 2012

(54) USES AND COMPOSITIONS FOR TREATMENT OF PSORIASIS AND CROHN'S DISEASE

(76) Inventors: Martin M. Okun, Libertyville, IL (US); Rebecca S. Hoffman, Wilmette, IL (US); Thomas C. Harris, Gurnee, IL (US); Parvez M. Mulani, Gurnee, IL (US); Jingdong Chao, Lake Bluff, IL (US); Talat Ashraf, Pleasanton, CA (US)

(21) Appl. No.: 13/346,215

(22) Filed: Jan. 9, 2012

Related U.S. Application Data
(63) Continuation of application No. 12/352,503, filed on Jan. 12, 2009, now abandoned.
Application No. 12/352,503 is a continuation of application No. 12/130,831, filed on May 30, 2008, now abandoned.
(60) Provisional application No. 60/932,914, filed on Jun. 1, 2007.
(60) Provisional application No. 61/011,538, filed on Jan. 17, 2008.
(60) Provisional application No. 61/024,122, filed on Jan. 28, 2008.
(60) Provisional application No. 61/128,498, filed on May 22, 2008.

(51) Int. Cl. G06F 19/24; A61P 1/00; A61K 39/395

(52) U.S. Cl. 424/142.1; 702/19

(57) ABSTRACT

The invention provides methods, uses and compositions for the treatment of psoriasis or Crohn's disease. The invention describes methods and uses for treating psoriasis or Crohn's disease, wherein a TNFα inhibitor, such as a human TNFα antibody, or antigen-binding portion thereof, is used to treat psoriasis in a subject. The invention includes methods of improving patient reported outcomes using a human human TNFα antibody, or antigen-binding portion thereof, for the treatment of Crohn's or psoriasis. The invention also provides methods of improving fatigue or depression in patients having Crohns'.

### RELATED APPLICATIONS

This application is a continuation of U.S. application Ser. No. 12/352,503, filed on Jan. 12, 2009, which is a continuation of U.S. application Ser. No. 12/130,831, filed on May 30, 2008, which claims the benefit of priority to U.S. provisional patent application No. 60/932,914 filed on Jun. 1, 2007; U.S. provisional patent application No. 61/011,538, filed Jan. 17, 2008; U.S. provisional patent application No. 61/024,122, filed Jan. 28, 2008; and U.S. provisional application No. 61/128,498, filed May 22, 2008. The contents of all the above-mentioned priority applications are hereby incorporated by reference in their entirety.

### BACKGROUND OF THE INVENTION

Psoriasis is a chronic, immune-mediated disease affecting 1-3% of the population worldwide (Jacobson and Kimball, Epidemiology: Psoriasis In: Psoriasis and Psoriatic Arthritis (Eds: Gordon K B, Ruderman E M). Springer-Verlag Berlin Heidelberg, Germany; 2005:47-56), with the greatest disease prevalence occurring in North America and Europe (Krueger and Duvic, J. Invest. Dermatol, 102:145-185, 1994). The most common form of psoriasis is plaque-type psoriasis, present in 65-86% of patients and characterized by the presence of thick, scaly plaques. Based on the National Psoriasis Foundation's definitions of moderate to severe psoriasis, the prevalence of moderate to severe psoriasis in the United States is estimated at 0.31% of persons age 18 or older (Stern et al., J. Investig. Dermatol. Symp. Proc. 9:136-139, 2004). Patients with psoriasis report reduction in physical functioning and mental functioning comparable to that observed in patients with cancer, arthritis, hypertension, heart disease, diabetes, and depression (Rapp et al., J. Am. Acad. Dermatol. 41(3N1):401-407, 1999). In a US survey of the impact of psoriasis on quality of life, respondents reported difficulties in the workplace, difficulties socializing with family members and friends, exclusion from public facilities, difficulties in getting a job, and contemplation of suicide (Krueger et al., Arch. Dermatol., 137:280-284, 2001).

Traditionally, treatment for psoriasis has included medications that suppress the growth of skin cells. Treatment approaches for psoriasis often include creams and ointments, oral medications, and phototherapy. In recent years, biologic response modifiers that inhibit certain cytokines have become a potential new avenue of treatment for psoriasis patients. For example, tumor necrosis factor (TNF) is a cytokine involved in inflammatory response and scientific evidence suggests it plays a fundamental role in the pathogenesis of psoriasis (Kreuger et al. (2004) Arch Dermatol 140:218; Kupper (2003) N Engl J Med 349:1987).

Crohn's disease is an inflammatory bowel disease, the general name for diseases that cause swelling in the intestines. For patients afflicted with Crohn's disease, the disease can have a devastating impact on their lifestyle, as common symptoms of Crohn's disease include diarrhea, cramping, abdominal pain, fever, and even rectal bleeding. Crohn's disease and complications associated with it often results in the patient requiring surgery, often more than once.

There is no known cure for Crohn's disease, and long-term, effective treatment options are limited. The goals of treatment are to control inflammation, correct nutritional deficiencies, and relieve symptoms like abdominal pain, diarrhea, and rectal to bleeding. While treatment can help control the disease by lowering the number of times a person experiences a recurrence, there is no cure. Treatment may include drugs, nutrition supplements, surgery, or a combination of these options. Common treatments which may be administered for treatment include anti-inflammation drugs, including sulfasalazine, cortisone or steroids, including prednisone, immune system suppressors, such as 6-mercaptopurine or azathioprine, and antibiotics.

Crohn's disease is a T-helper Type 1 (Th 1) disease, which has an immune response pattern that includes an increased production of interleukin-12, tumour necrosis factor (TNF), and interferon γ (Romagnani. Inflamm Bowel Dis 1999; 5:285-94). Increased production of TNF by macrophages in patients with Crohn's disease (CD) results in elevated concentrations of TNF in the stool, blood, and mucosa (Murch et al. Gut 1991; 32:913-7; Braegger et al. Lancet 1992; 339:89-91; Murch et al. Gut 1993; 34:1705-9). Tumor necrosis factor (TNF) has been identified as an important cytokine in the pathogenesis of Crohn's disease (CD), with elevated concentrations playing a role in pathologic inflammation (Papadakis et al. Gastroenterology 2000; 119:1148-1157; Van Deventer Gut 1997; 40:443-448). In recent years biologic response modifiers that inhibit TNF activity have become potential therapies for treating Crohn's disease.

### SUMMARY OF THE INVENTION

There remains a need for an effective and safe treatment option for patients suffering from psoriasis and Crohn's disease and Crohn's related disorders. There also remains a need for improved methods and compositions that provide a safe and effective treatment of psoriasis and CD using TNFα inhibitors.

The instant invention provides improved methods and compositions for treating psoriasis and CD. The invention further provides a means for treating certain subpopulations of patients who have psoriasis or CD. The invention further provides a means by which the efficacy of a TNFα inhibitor for the treatment of psoriasis or CD can be determined. The invention also includes methods for treating certain types of psoriasis or CD, e.g., early CD. The invention further provides methods for identifying subjects having psoriasis or CD who will benefit from TNF antagonist therapy. Kits and labels which provide information pertaining to the methods, uses, and compositions of the invention are also described herein. Each of the examples described herein describes methods and compositions which can be used to determine whether a TNFα inhibitor is effective for treating the given disorder.

Accordingly, in one aspect the invention provides a method of determining the efficacy of a TNFα inhibitor for achieving a clinical response in Crohn's disease in a subject comprising, determining the HRQL of a patient population having Crohn's to disease and who were administered the TNFα inhibitor, wherein a statistically significant improvement in the HRQL of the patient population indicates that the TNFα inhibitor is an effective for achieving a clinical response in Crohn's disease in a subject.

In another aspect, the invention provides a method of determining the efficacy of a TNFα □inhibitor for maintaining remission of Crohn's disease in a subject comprising determining the HRQL of a patient population having Crohn's disease and who were administered the TNFα □inhibitor inhibitor, wherein a statistically significant improvement in the HRQL of the patient population indicates that the TNFα □inhibitor □inhibitor is an effective for maintaining remission of Crohn's disease in a subject.

In certain embodiments of these aspects of the invention, determining the HRQL comprises using one or more Patient Related Outcome scores or scales selected from the group consisting of IBDQ score, SF-36 PCS score, SF-36 MCS score, FACIT-fatigue score, Zung depression score, VAS score, and a combination thereof.

In one embodiment, a mean increase of 5 or more points in the IBDQ score of the patient population indicates that the TNFα □ inhibitor is effective for achieving a clinical response in Crohn's disease in a subject and/or maintaining remission of Crohn's disease in a subject. In another embodiment, a mean increase of 7 or more points in the IBDQ score of the patient population indicates that the TNFα □inhibitor □inhibitor is effective for achieving a clinical response in Crohn's disease in a subject and/or maintaining remission of Crohn's disease in a subject.

In one embodiment, a mean increase of 3 or more points in the SF-36 MCS of the patient population indicates that the TNFα inhibitor is effective for achieving a clinical response in Crohn's disease in a subject and/or maintaining remission of Crohn's disease in a subject. In another embodiment a mean increase of 5 or more points in the SF-36 MCS of the patient population indicates that the TNFα inhibitor is effective for achieving a clinical response in Crohn's disease in a subject and/or maintaining remission of Crohn's disease in a subject. In another embodiment a mean increase of 8 or more points in the SF-36 MCS of the patient population indicates that the TNFα inhibitor is effective for achieving a clinical response in Crohn's disease in a subject and/or maintaining remission of Crohn's disease in a subject. In another embodiment, a mean increase of 10 or more points in the SF-36 MCS of the patient population indicates that the TNFα inhibitor is effective for achieving a clinical response in Crohn's disease in a subject and/or maintaining remission of Crohn's disease in a subject.

In one embodiment, a mean increase of 3 or more points in the SF-36 PCS score of the patient population indicates that the TNFα inhibitor is effective for achieving a clinical response in Crohn's disease in a subject and/or maintaining remission of Crohn's disease in a subject. In another embodiment, a mean increase of 5 or more points in the SF-36 PCS score of the patient population indicates that the TNFα inhibitor is effective for achieving a clinical response in Crohn's disease in a subject and/or maintaining remission of Crohn's disease in a subject. In another embodiment, a mean increase of 8 or more points in the SF-36 PCS score of the patient population indicates that the TNFα inhibitor is effective for achieving a clinical response in Crohn's disease in a subject and/or maintaining remission of Crohn's disease in a subject.

In one embodiment, a mean increase of 3 or more points in the FACIT-fatigue score of the patient population indicates that the TNFα inhibitor is effective for achieving a clinical response in Crohn's disease in a subject and/or maintaining remission of Crohn's disease in a subject. In another embodiment, a mean increase of 10 or more points in the FACIT-fatigue score of the patient population indicates that the TNFα inhibitor is effective for achieving a clinical response in Crohn's disease in a subject.

In one embodiment, a mean decrease of 5 or more points in the Zung depression score of the patient population indicates that the TNFα inhibitor is effective for achieving a clinical response in Crohn's disease in a subject and/or maintaining remission of Crohn's disease in a subject. In another embodiment a mean decrease of 9 or more points in the Zung depression score of the patient population indicates that the TNFα inhibitor is effective for achieving a clinical response in Crohn's disease in a subject and/or maintaining remission of Crohn's disease in a subject.

In one embodiment, a mean increase of 4 or more points in the mean VAS score of the patient population indicates that the TNFα inhibitor is effective for achieving a clinical response in Crohn's disease in a subject and/or maintaining remission of Crohn's disease in a subject.

In one embodiment, the methods of the invention comprise determining the Crohn's Disease Activity Index (CDAI) score of the patient population, wherein a decreast of at least 70 in the CDAI score of at least 43% of the patient population indicates that the TNFα inhibitor is effective for achieving a clinical response in Crohn's disease in a subject and/or maintaining remission of Crohn's disease in a subject.

In another embodiment, the methods of the invention further comprise administering an effective TNFα inhibitor to the subject to achieve a clinical response to Crohn's disease, and/or maintaining remission of Crohn's disease in a subject.

In certain embodiments of the methods of the invention, the TNFα inhibitor is a human TNFα antibody, or an antigen binding portion thereof, and wherein dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less. In related embodiments, the human TNFα antibody, or an antigen-binding portion thereof, has the following characteristics:

a) dissociates from human TNFα with a K_{off} rate constant of 1×10⁻³ s⁻¹ or less, as determined by surface plasmon resonance;

b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;

c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

In another embodiment, the human TNFα antibody, or an antigen-binding portion thereof, comprises a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and comprises a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11.

In another embodiment, the human TNFα antibody, or an antigen-binding portion thereof, comprises a light chain variable region (LCVR) comprising the amino acid sequence of SEQ ID NO: 1 and a heavy chain variable region (HCVR) comprising the amino acid sequence of SEQ ID NO: 2.

In another embodiment, the human TNFα antibody, or an antigen-binding portion thereof, is adalimumab. The another embodiment of the invention, the anti-TNFα □antibody, or antigen-binding portion thereof, is infliximab or golimumab.

The invention further provides a method for improving fatigue in a patient having Crohn's disease comprising administering a human TNFα antibody, or antigen-binding portion thereof, to the patient, such that fatigue is improved. In one embodiment, the FACIT score of the patient is improved.

The invention also provides a method for treating depression in a patient having Crohn's disease comprising administering a human TNFα antibody, or antigen-binding portion thereof, to the patient, such that depression is treated.

In another aspect, the invention provides an article of manufacture comprising a human TNFα antibody, or antigen-binding portion thereof, and a label or package insert, wherein the label or package insert indicates that the human TNFα antibody, or antigen-binding portion thereof, may be used for the treatment of adult patients with Crohn's disease.

In another aspect, the invention provides an article of manufacture comprising a human TNFα antibody, or antigen-binding portion thereof, and a label or package insert, wherein the label or package insert indicates that the human TNFα antibody, or antigen-binding portion thereof, may be used for the treatment of adult patients with moderate to severe chronic plaque psoriasis.

### DETAILED DESCRIPTION OF THE INVENTION

### I. Definitions

The term “human TNFα” (abbreviated herein as hTNFα, or simply hTNF), as used herein, is intended to refer to a human cytokine that exists as a 17 kD secreted form and a 26 kD membrane associated form, the biologically active form of which is composed of a trimer of noncovalently bound 17 kD molecules. The structure of hTNFα is described further in, for example, Pennica, D., et al. (1984) Nature 312:724-729; Davis, J. M., et al. (1987) Biochemistry 26:1322-1326; and Jones, E. Y., et al. (1989) Nature 338:225-228. The term human TNFα is intended to include recombinant human TNFα (rhTNFα), which can be prepared by standard recombinant expression methods or purchased commercially (R & D Systems, Catalog No. 210-TA, Minneapolis, Minn.). TNFα is also referred to as TNF.

The term “TNFα inhibitor” includes agents which interfere with TNFα activity. The term also includes each of the anti-TNFα human antibodies and antibody portions described herein as well as those described in U.S. Pat. Nos. 6,090,382; 6,258,562; 6,509,015, and in U.S. patent application Ser. Nos. 09/801,185 and 10/302,356. In one embodiment, the TNFα inhibitor used in the invention is an anti-TNFα antibody, or a fragment thereof, including infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (HUMIRA®® Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, each of which is incorporated by reference herein. In another embodiment, the TNFα inhibitor is a TNF fusion protein, e.g., etanercept (Enbrel®, Amgen; described in WO 91/03553 and WO 09/406,476, incorporated by reference herein). In another embodiment, the TNFα inhibitor is a to recombinant TNF binding protein (r-TBP-I) (Serono).

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The antibodies of the invention are described in further detail in U.S. Pat. Nos. 6,090,382; 6,258,562; and 6,509,015, each of which is incorporated herein by reference in its entirety.

The term “antigen-binding portion” or “antigen-binding fragment” of an antibody (or simply “antibody portion”), as used herein, refers to one or more fragments of an antibody that retain the ability to specifically bind to an antigen (e.g., hTNFα). It has been shown that the antigen-binding function of an antibody can be performed by fragments of a full-length antibody. Binding fragments include Fab, Fab′, F(ab′)₂, Fabc, Fv, single chains, and single-chain antibodies. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, to but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. USA 90:6444-6448; Poljak et al. (1994) Structure 2:1121-1123). The antibody portions of the invention are described in further detail in U.S. Pat. Nos. 6,090,382, 6,258,562, 6,509,015, each of which is incorporated herein by reference in its entirety.

Still further, an antibody or antigen-binding portion thereof may be part of a larger immunoadhesion molecules, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

A “conservative amino acid substitution”, as used herein, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Chimeric antibodies” refers to antibodies wherein one portion of each of the amino acid sequences of heavy and light chains is homologous to corresponding sequences in antibodies derived from a particular species or belonging to a particular class, while the remaining segment of the chains is homologous to corresponding sequences from another species. In one embodiment, the invention features a chimeric antibody or antigen-binding fragment, in which the variable regions of both light and heavy chains mimics the variable regions of antibodies derived from one species of mammals, while the constant portions are homologous to the sequences in antibodies derived from another species. In a preferred embodiment of the invention, chimeric to antibodies are made by grafting CDRs from a mouse antibody onto the framework regions of a human antibody.

“Humanized antibodies” refer to antibodies which comprise at least one chain comprising variable region framework residues substantially from a human antibody chain (referred to as the acceptor immunoglobulin or antibody) and at least one complementarity determining region (CDR) substantially from a non-human-antibody (e.g., mouse). In addition to the grafting of the CDRs, humanized antibodies typically undergo further alterations in order to improve affinity and/or immunogenicity.

The term “multivalent antibody” refers to an antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” or “dual specific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Such chimeric, humanized, human, and dual specific antibodies can be produced by recombinant DNA techniques known in the art, for example using methods described in PCT International Application No. PCT/US86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060, Queen et al., Proc. Natl. Acad. Sci. USA 86:10029-10033 (1989), U.S. Pat. No. 5,530,101, U.S. Pat. No. 5,585,089, U.S. Pat. No. 5,693,761, U.S. Pat. No. 5,693,762, Selick et al., WO 90/07861, and Winter, U.S. Pat. No. 5,225,539.

An “isolated antibody”, as used herein, is intended to refer to an antibody that is substantially free of other antibodies having different antigenic specificities (e.g., an isolated antibody that specifically binds hTNFα is substantially free of antibodies that specifically_bind antigens other than hTNFα). An isolated antibody that specifically binds hTNFα may, however, have cross-reactivity to other antigens, such as TNFα molecules from other species. Moreover, an isolated antibody may be substantially free of other cellular material and/or chemicals.

A “neutralizing antibody”, as used herein (or an “antibody that neutralized hTNFα activity”), is intended to refer to an antibody whose binding to hTNFα results in inhibition of the biological activity of hTNFα. This inhibition of the biological activity of hTNFα can be assessed by measuring one or more indicators of hTNFα biological activity, such as hTNFα-induced cytotoxicity (either in vitro or in vivo), hTNFα-induced cellular activation and hTNFα binding to hTNFα receptors. These indicators of hTNFα biological activity can be assessed by one or more of several standard in vitro or in vivo assays known in the art (see U.S. Pat. No. 6,090,382). Preferably, the ability of an antibody to neutralize hTNFα activity is assessed by inhibition of hTNFα-induced cytotoxicity of L929 cells. As an additional or alternative parameter of hTNFα activity, the ability of an antibody to inhibit hTNFα-induced expression of ELAM-1 on HUVEC, as a measure of hTNFα-induced cellular activation, can be assessed.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 1 of U.S. Pat. No. 6,258,562 and Jonsson et al. (1993) Ann. Biol. Clin. 51:19; Jonsson et al. (1991) Biotechniques 11:620-627; Johnsson et al. (1995) J. Mol. Recognit. 8:125; and Johnnson et al. (1991) Anal. Biochem. 198:268.

The term “K_{off}”, as used herein, is intended to refer to the off rate constant for dissociation of an antibody from the antibody/antigen complex.

The term “K_{d}”, as used herein, is intended to refer to the dissociation constant of a particular antibody-antigen interaction.

The term “IC₅₀” as used herein, is intended to refer to the concentration of the inhibitor required to inhibit the biological endpoint of interest, e.g., neutralize cytotoxicity activity.

The term “dose,” as used herein, refers to an amount of TNFα inhibitor which is administered to a subject.

The term “dosing”, as used herein, refers to the administration of a substance (e.g., an anti-TNFα antibody) to achieve a therapeutic objective (e.g., treatment of psoriasis).

A “dosing regimen” describes a treatment schedule for a TNFα inhibitor, e.g., a treatment schedule over a prolonged period of time and/or throughout the course of treatment, e.g. administering a first dose of a TNFα inhibitor at week 0 followed by a second dose of a TNFα inhibitor on a biweekly dosing regimen.

The term “multiple-variable dose” includes different doses of a TNFα inhibitor which are administered to a subject for therapeutic treatment. “Multiple-variable dose regimen” or “multiple-variable dose therapy” describes a treatment schedule which is based on administering different amounts of TNFα inhibitor at various time points throughout the course of treatment. Multiple-variable dose regimens are described in U.S. patent application Ser. No. 11/104,117, filed Apr. 11, 2005 (US 20060009385) and PCT application no. PCT/US05/12007, which are incorporated by reference herein.

The term “maintenance therapy” or “maintenance dosing regime” refers to a treatment schedule for a subject or patient diagnosed with a disorder/disease, e.g., to psoriasis, to enable them to maintain their health in a given state, e.g, remission. Generally, the first goal of treatment of psoriasis is to induce remission in the subject in need thereof. The next challenge is to keep the subject in remission. Maintenance doses may be used in a maintenance therapy for maintaining remission in a subject who has achieved remission of a disease or who has reached a state of the disease which is advantageous, e.g. reduction in symptoms. In one embodiment, a maintenance therapy of the invention is used for a subject or patient diagnosed with a disorder/disease, e.g., psoriasis to enable them to maintain their health in a state which is completely free of symptoms associated with the disease. In one embodiment, a maintenance therapy of the invention is used for a subject or patient diagnosed with a disorder/disease, e.g., psoriasis, to enable them to maintain their health in a state which is substantially free of symptoms associated with the disease. In one embodiment, a maintenance therapy of the invention is used for a subject or patient diagnosed with a disorder/disease, e.g., psoriasis, to enable them to maintain their health in a state where there is a significant reduction in symptoms associated with the disease.

The term “induction dose” or “loading dose,” used interchangeably herein, refers to the first dose of TNFα inhibitor which is initially used to induce remission of psoriasis. Often, the loading dose is larger in comparison to the subsequent maintenance or treatment dose. The induction dose can be a single dose or, alternatively, a set of doses. In one embodiment, an induction dose is subsequently followed by administration of smaller doses of TNFα inhibitor, e.g., the treatment or maintenance dose. The induction dose is administered during the induction or loading phase of therapy. In one embodiment of the invention, the induction dose is at least twice the given amount of the treatment dose. In one embodiment of the invention, the induction dose is 80 mg. In another embodiment, the induction dose is 160 mg.

The term “treatment phase” or “maintenance phase”, as used herein, refers to a period of treatment comprising administration of a TNFα inhibitor to a subject in order to maintain a desired therapeutic effect, i.e., maintaining remission of psoriasis.

The term “maintenance dose” or “treatment dose” is the amount of TNFα inhibitor taken by a subject to maintain or continue a desired therapeutic effect. A maintenance dose can be a single dose or, alternatively, a set of doses. A maintenance dose is administered during the treatment or maintenance phase of therapy. In one embodiment, a maintenance dose(s) is smaller than the induction dose(s) and can be equal to each other when administered in succession. In one embodiment, the invention provides a maintenance dose of 40 mg of adalimumab administered subcutaneously to a subject who is in remission, every other week, or biweekly. In one embodiment, the maintenance dose is administered every other week beginning at week 4 of treatment.

The terms “biweekly dosing regimen”, “biweekly dosing”, and “biweekly administration”, as used herein, refer to the time course of administering a substance (e.g., an anti-TNFα antibody) to a subject to achieve a therapeutic objective, e.g, throughout the course of treatment. The biweekly dosing regimen is not intended to include a weekly dosing regimen. Preferably, the substance is administered every 9-19 days, more preferably, every 11-17 days, even more preferably, every 13-15 days, and most preferably, every 14 days. In one embodiment, the biweekly dosing regimen is initiated in a subject at week 0 of treatment. In another embodiment, a maintenance dose is administered on a biweekly dosing regimen. In one embodiment, both the loading and maintenance doses are administered according to a biweekly dosing regimen. In one embodiment, biweekly dosing includes a dosing regimen wherein doses of a TNFα inhibitor are administered to a subject every other week beginning at week 0. In one embodiment, biweekly dosing includes a dosing regimen where doses of a TNFα inhibitor are administered to a subject every other week consecutively for a given time period, e.g., 4 weeks, 8 weeks, 16, weeks, 24 weeks, 26 weeks, 32 weeks, 36 weeks, 42 weeks, 48 weeks, 52 weeks, 56 weeks, etc. Biweekly dosing methods are also described in US 20030235585, incorporated by reference herein.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The term “combination therapy”, as used herein, refers to the administration of to two or more therapeutic substances, e.g., an anti-TNFα antibody and another drug. The other drug(s) may be administered concomitant with, prior to, or following the administration of an anti-TNFα antibody.

The term “treatment,” as used within the context of the present invention, is meant to include therapeutic treatment, as well as prophylactic or suppressive measures, for the treatment of psoriasis. For example, the term treatment may include administration of a TNFα inhibitor prior to or following the onset of psoriasis thereby preventing or removing signs of the disease or disorder. As another example, administration of a TNFα inhibitor after clinical manifestation of psoriasis or Crohn's disease to combat the symptoms and/or complications and disorders associated with psoriasis or Crohn's disease comprises “treatment” of the disease. Further, administration of the agent after onset and after clinical symptoms and/or complications have developed where administration affects clinical parameters of the disease or disorder and perhaps amelioration of the disease, comprises “treatment” of the psoriasis or Crohn's disease. In one embodiment, treatment of psoriasis in a subject comprises inducing and maintaining remission of psoriasis or Crohn's disease in a subject. In another embodiment, treatment of psoriasis or Crohn's disease in a subject comprises maintaining remission of psoriasis or Crohn's disease in a subject.

Those “in need of treatment” include mammals, such as humans, already having psoriasis, including those in which the disease or disorder is to be prevented.

“HRQL” or “QOL” refer to a patent's subjective evaluations of the influences of their current health status, health care, and health promoting activities on their perceived physical and mental health over time. Changes in the HQRL or QOL of a patient represent the functional effects of an illness (e.g., Crohn's disease) and consequent therapy (e.g., TNFα inhibitor therapy) upon the patient, as perceived by the patient. The HQRL or QOL of a patient or patient population can be determined using one or more measures of patient-related outcomes (PRO).

The term “patient reported outcome” or “PRO”, as used herein, refers to information provided by the patient. For example, a patient or group of patients, or a potential patient or former patient or a group of such individuals, may be asked to complete one or more questionnaires. In one embodiment, a patient or group of patients may be asked to complete a self-administered questionnaire. In one embodiment, a patient or group of patients may be asked to participate in an interviewer-administered questionnaire, wherein the interviewer gains the patient's views. In other embodiments, the patient or group of patients may be asked to complete a combination of questionnaires. Other known terms in the art for a PRO questionnaire are “instrument”, “measure”, “scale” and “tool”. A PRO may assess a single characteristic or multiple characteristics. In some embodiments, a PRO inquires about symptoms and/impairments, functioning and/or disability, health related quality of life, and/or quality of life. A PRO may be generic, in that it can provide information regarding a wide variety of diseases or conditions. An instrument may be condition-specific or specific to a set of diseases or disorders with common symptoms, organs, organ systems or tissues. Examples of PRO methods for determining HQRL or QOL according to the methods of the invention include, but are not limited to, IBDQ score, SF-36 score, SF-12 score, WKAI score, FACIT-f score, Zung Depression score and VAS score.

Various aspects of the invention are described in further detail herein.

The invention provides improved uses and compositions for treating psoriasis or Crohn's disease with a TNFα inhibitor, e.g., a human TNFα antibody, or an antigen-binding portion thereof. Compositions and articles of manufacture, including kits, relating to the methods and uses for treating psoriasis or Crohn's disease are also contemplated as part of the invention.

### II. TNF Inhibitors

A TNFα inhibitor which is used in the methods and compositions of the invention includes any agent which interferes with TNFα activity. In a preferred embodiment, the TNFα inhibitor can neutralize TNFα activity, particularly detrimental TNFα activity which is associated with psoriasis, and related complications and symptoms.

In one embodiment, the TNFα inhibitor used in the invention is an TNFα antibody (also referred to herein as a TNFα antibody), or an antigen-binding fragment thereof, including chimeric, humanized, and human antibodies. Examples of TNFα antibodies which may be used in the invention include, but not limited to, infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab; Medarex and Centocor, see WO 02/12502), and adalimumab (HUMIRA® Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, each of which is incorporated by reference herein.

Other examples of TNFα inhibitors which may be used in the methods and compositions of the invention include etanercept (Enbrel, described in WO 91/03553 and WO 09/406,476), soluble TNF receptor Type I, a pegylated soluble TNF receptor Type I (PEGs TNF-R1), p55TNFR1gG (Lenercept), and recombinant TNF binding protein (r-TBP-I) (Serono).

In one embodiment, the term “TNFα inhibitor” excludes infliximab. In one embodiment, the term “TNFα inhibitor” excludes adalimumab. In another embodiment, the term “TNFα inhibitor” excludes adalimumab and infliximab.

In one embodiment, the term “TNFα inhibitor” excludes etanercept, and, optionally, adalimumab, infliximab, and adalimumab and infliximab.

In one embodiment, the term “TNFα antibody” excludes infliximab. In one embodiment, the term “TNFα antibody” excludes adalimumab. In another embodiment, the term “TNFα antibody” excludes adalimumab and infliximab.

In one embodiment, the invention features uses and compositions for treating or determining the efficacy of a TNFα inhibitor for the treatment of psoriasis or Crohn's disease, wherein the TNFα antibody is an isolated human antibody, or antigen-binding portion thereof, that binds to human TNFα with high affinity and a low off rate, and also has a high neutralizing capacity. Preferably, the human antibodies used in the invention are recombinant, neutralizing human anti-hTNFα antibodies. The most preferred recombinant, neutralizing antibody of the invention is referred to herein as D2E7, also referred to as HUMIRA® or adalimumab (the amino acid sequence of the D2E7 VL region is shown in SEQ ID NO: 1; the amino acid sequence of the D2E7 VH region is shown in SEQ ID NO: 2). The properties of D2E7 (adalimumab/HUMIRA®) have been described in Salfeld et al., U.S. Pat. Nos. 6,090,382, 6,258,562, and 6,509,015, which are each incorporated by reference herein. The methods of the invention may also be performed using chimeric and humanized murine anti-hTNFα antibodies which have undergone clinical testing for treatment of rheumatoid arthritis (see e.g., Elliott, M. J., et al. (1994) Lancet 344:1125-1127; Elliot, M. J., et al. (1994) Lancet 344:1105-1110; Rankin, E. C., et al. (1995) Br. J. Rheumatol. 34:334-342).

In one embodiment, the method of the invention includes determining the efficacy of D2E7 antibodies and antibody portions, D2E7-related antibodies and antibody portions, or other human antibodies and antibody portions with equivalent properties to D2E7, such as high affinity binding to hTNFα with low dissociation kinetics and high neutralizing capacity, for the treatment of psoriasis. In one embodiment, the invention provides treatment with an isolated human antibody, or an antigen-binding portion thereof, that dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less. More preferably, the isolated human antibody, or antigen-binding portion thereof, dissociates from human TNFα with a K_{off} of 5×10⁻⁴ s⁻¹ or less, or even more preferably, with a K_{off} of 1×10⁻⁴ s⁻¹ or less. More preferably, the isolated human antibody, or antigen-binding portion thereof, neutralizes human TNFα cytotoxicity in a standard in vitro L929 assay with an IC₅₀ of to 1×10⁻⁸ M or less, even more preferably with an IC₅₀ of 1×10⁻⁹ M or less and still more preferably with an IC₅₀ of 1×10⁻¹⁰ M or less. In a preferred embodiment, the antibody is an isolated human recombinant antibody, or an antigen-binding portion thereof.

It is well known in the art that antibody heavy and light chain CDR3 domains play an important role in the binding specificity/affinity of an antibody for an antigen. Accordingly, in another aspect, the invention pertains to treating psoriasis or Crohn's disease by administering human antibodies that have slow dissociation kinetics for association with hTNFα and that have light and heavy chain CDR3 domains that structurally are identical to or related to those of D2E7. Position 9 of the D2E7 VL CDR3 can be occupied by Ala or Thr without substantially affecting the K_{off}.

Accordingly, a consensus motif for the D2E7 VL CDR3 comprises the amino acid sequence: Q-R-Y-N-R-A-P-Y-(T/A) (SEQ ID NO: 3). Additionally, position 12 of the D2E7 VH CDR3 can be occupied by Tyr or Asn, without substantially affecting the K_{off}. Accordingly, a consensus motif for the D2E7 VH CDR3 comprises the amino acid sequence: V-S-Y-L-S-T-A-S-S-L-D-(Y/N) (SEQ ID NO: 4). Moreover, as demonstrated in Example 2 of U.S. Pat. No. 6,090,382, the CDR3 domain of the D2E7 heavy and light chains is amenable to substitution with a single alanine residue (at position 1, 4, 5, 7 or 8 within the VL CDR3 or at position 2, 3, 4, 5, 6, 8, 9, 10 or 11 within the VH CDR3) without substantially affecting the K_{off}. Still further, the skilled artisan will appreciate that, given the amenability of the D2E7 VL and VH CDR3 domains to substitutions by alanine, substitution of other amino acids within the CDR3 domains may be possible while still retaining the low off rate constant of the antibody, in particular substitutions with conservative amino acids. Preferably, no more than one to five conservative amino acid substitutions are made within the D2E7 VL and/or VH CDR3 domains. More preferably, no more than one to three conservative amino acid substitutions are made within the D2E7 VL and/or VH CDR3 domains. Additionally, conservative amino acid substitutions should not be made at amino acid positions critical for binding to hTNFα. Positions 2 and 5 of the D2E7 VL CDR3 and positions 1 and 7 of the D2E7 VH CDR3 appear to be critical for interaction with hTNFα and thus, conservative amino acid substitutions preferably are not made at these positions (although an alanine substitution at position 5 of the D2E7 VL CDR3 is acceptable, as described above) (see U.S. Pat. No. 6,090,382).

Accordingly, in another embodiment, the antibody or antigen-binding portion thereof preferably contains the following characteristics:

a) dissociates from human TNFα with a K_{off} rate constant of 1×10⁻³ s⁻¹ or less, as determined by surface plasmon resonance;

b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;

c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

More preferably, the antibody, or antigen-binding portion thereof, dissociates from human TNFα with a K_{off} of 5×10⁻⁴ s⁻¹ or less. Even more preferably, the antibody, or antigen-binding portion thereof, dissociates from human TNFα with a K_{off} of 1×10⁻⁴ s⁻¹ or less.

In yet another embodiment, the antibody or antigen-binding portion thereof preferably contains a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and with a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11. Preferably, the LCVR further has a CDR2 domain comprising the amino acid sequence of SEQ ID NO: 5 (i.e., the D2E7 VL CDR2) and the HCVR further has a CDR2 domain comprising the amino acid sequence of SEQ ID NO: 6 (i.e., the D2E7 VH CDR2). Even more preferably, the LCVR further has CDR1 domain comprising the amino acid sequence of SEQ ID NO: 7 (i.e., the D2E7 VL CDR1) and the HCVR has a CDR1 domain comprising the amino acid sequence of SEQ ID NO: 8 (i.e., the D2E7 VH CDR1). The framework regions for VL preferably are from the V_{K}I human germline family, more preferably from the A20 human germline Vk gene and most preferably from the D2E7 VL framework sequences shown in FIGS. 1A and 1B of U.S. Pat. No. 6,090,382. The framework regions for VH preferably are from the V_{H}3 human germline family, more preferably from the DP-31 human germline VH gene and most preferably from the D2E7 VH framework sequences shown in FIGS. 2A and 2B of U.S. Pat. No. 6,090,382.

Accordingly, in another embodiment, the antibody or antigen-binding portion thereof preferably contains a light chain variable region (LCVR) comprising the amino acid sequence of SEQ ID NO: 1 (i.e., the D2E7 VL) and a heavy chain variable region (HCVR) comprising the amino acid sequence of SEQ ID NO: 2 (i.e., the D2E7 VH). In certain embodiments, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

In still other embodiments, the invention includes uses of an isolated human antibody, or an antigen-binding portions thereof, containing D2E7-related VL and VH CDR3 domains. For example, antibodies, or antigen-binding portions thereof, with a light chain variable region (LCVR) having a CDR3 domain comprising an amino acid sequence selected from the group consisting of SEQ ID NO: 3, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25 and SEQ ID NO: 26 or with a heavy chain variable region (HCVR) having a CDR3 domain comprising an amino acid sequence selected from the group consisting of SEQ ID NO: 4, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 and SEQ ID NO: 35.

The TNFα antibody used in the methods and compositions of the invention may be modified for improved treatment of psoriasis or Crohn's disease. In some embodiments, the TNFα antibody or antigen binding fragments thereof, is chemically modified to provide a desired effect. For example, pegylation of antibodies and antibody fragments of the invention may be carried out by any of the pegylation reactions known in the art, as described, for example, in the following references: Focus on Growth Factors 3:4-10 (1992); EP 0 154 316; and EP 0 401 384 (each of which is incorporated by reference herein in its entirety). Preferably, the pegylation is carried out via an acylation reaction or an alkylation reaction with a reactive polyethylene glycol molecule (or an analogous reactive water-soluble polymer). A preferred water-soluble polymer for pegylation of the antibodies and antibody fragments of the invention is polyethylene glycol (PEG). As used herein, “polyethylene glycol” is meant to encompass any of the forms of PEG that have been used to derivatize other proteins, such as mono (Cl—ClO) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

Pegylated antibodies and antibody fragments may generally be used to treat psoriasis by administration of the TNFα antibodies and antibody fragments described herein. Generally the pegylated antibodies and antibody fragments have increased half-life, as compared to the nonpegylated antibodies and antibody fragments. The pegylated antibodies and antibody fragments may be employed alone, together, or in combination with other pharmaceutical compositions.

In yet another embodiment of the invention, TNFα antibodies or fragments thereof can be altered wherein the constant region of the antibody is modified to reduce at least one constant region-mediated biological effector function relative to an unmodified antibody. To modify an antibody of the invention such that it exhibits reduced binding to the Fc receptor, the immunoglobulin constant region segment of the antibody can be mutated at particular regions necessary for Fc receptor (FcR) interactions (see e.g., Canfield, S. M. and S. L. Morrison (1991) J. Exp. Med. 173:1483-1491; and Lund, J. et al. (1991) J. of Immunol. 147:2657-2662). Reduction in FcR binding ability of the antibody may also reduce other effector functions which rely on FcR interactions, such as opsonization and phagocytosis and antigen-dependent cellular cytotoxicity.

An antibody or antibody portion used in the methods of the invention can be derivatized or linked to another functional molecule (e.g., another peptide or protein). Accordingly, the antibodies and antibody portions of the invention are intended to include derivatized and otherwise modified forms of the human anti-hTNFα antibodies described herein, including immunoadhesion molecules. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody or antibody portion of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

An antibody, or antibody portion, used in the methods and compositions of the invention, can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

To express adalimumab (D2E7) or an adalimumab (D2E7)-related antibody, DNA fragments encoding the light and heavy chain variable regions are first obtained. These DNAs can be obtained by amplification and modification of germline light and heavy chain variable sequences using the polymerase chain reaction (PCR). Germline DNA sequences for human heavy and light chain variable region genes are known in the art (see e.g., the “Vbase” human germline sequence database; see also Kabat, E. A., et al. (1991) Sequences of Proteins of Immunological Interest, Fifth Edition, U.S. Department of Health and Human Services, NIH Publication No. 91-3242; Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J.P. L. et al. (1994) “A Directory of Human Germ-line V₇₈ Segments Reveals a Strong Bias in their Usage” Eur. J. Immunol. 24:827-836; the contents of each of which are expressly incorporated herein by reference). To obtain a DNA fragment encoding the heavy chain variable region of D2E7, or a D2E7-related antibody, a member of the V_{H}3 family of human germline VH genes is amplified by standard PCR. Most to preferably, the DP-31 VH germline sequence is amplified. To obtain a DNA fragment encoding the light chain variable region of D2E7, or a D2E7-related antibody, a member of the V_{κ}I family of human germline VL genes is amplified by standard PCR. Most preferably, the A20 VL germline sequence is amplified. PCR primers suitable for use in amplifying the DP-31 germline VH and A20 germline VL sequences can be designed based on the nucleotide sequences disclosed in the references cited supra, using standard methods.

Once the germline VH and VL fragments are obtained, these sequences can be mutated to encode the D2E7 or D2E7-related amino acid sequences disclosed herein. The amino acid sequences encoded by the germline VH and VL DNA sequences are first compared to the D2E7 or D2E7-related VH and VL amino acid sequences to identify amino acid residues in the D2E7 or D2E7-related sequence that differ from germline. Then, the appropriate nucleotides of the germline DNA sequences are mutated such that the mutated germline sequence encodes the D2E7 or D2E7-related amino acid sequence, using the genetic code to determine which nucleotide changes should be made. Mutagenesis of the germline sequences is carried out by standard methods, such as PCR-mediated mutagenesis (in which the mutated nucleotides are incorporated into the PCR primers such that the PCR product contains the mutations) or site-directed mutagenesis.

Moreover, it should be noted that if the “germline” sequences obtained by PCR amplification encode amino acid differences in the framework regions from the true germline configuration (i.e., differences in the amplified sequence as compared to the true germline sequence, for example as a result of somatic mutation), it may be desirable to change these amino acid differences back to the true germline sequences (i.e., “backmutation” of framework residues to the germline configuration).

Once DNA fragments encoding D2E7 or D2E7-related VH and VL segments are obtained (by amplification and mutagenesis of germline VH and VL genes, as described above), these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (CH1, CH2 and CH3). The sequences to of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region, but most preferably is a kappa constant region.

To create a scFv gene, the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, or antibody portions used in the invention, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Prior to insertion of the D2E7 or D2E7-related light or heavy chain sequences, the expression vector may already carry antibody constant region sequences. For example, one approach to converting the D2E7 or D2E7-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In addition to the antibody chain genes and regulatory sequences, the recombinant expression vectors used in the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It is understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to hTNFα. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than hTNFα by crosslinking an antibody of the invention to a second antibody by standard chemical crosslinking methods.

In a preferred system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are culture to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

In view of the foregoing, nucleic acid, vector and host cell compositions that can be used for recombinant expression of the antibodies and antibody portions used in the invention include nucleic acids, and vectors comprising said nucleic acids, comprising the human TNFα antibody adalimumab (D2E7). The nucleotide sequence encoding the D2E7 light chain variable region is shown in SEQ ID NO: 36. The CDR1 domain of the LCVR encompasses nucleotides 70-102, the CDR2 domain encompasses nucleotides 148-168 and the CDR3 domain encompasses nucleotides 265-291. The nucleotide sequence encoding the D2E7 heavy chain variable region is shown in SEQ ID NO: 37. The CDR1 domain of the HCVR encompasses nucleotides 91-105, the CDR2 domain encompasses nucleotides 148-198 and the CDR3 domain encompasses nucleotides 295-330. It will be appreciated by the skilled artisan that nucleotide sequences encoding D2E7-related antibodies, or portions thereof (e.g., a CDR domain, such as a CDR3 domain), can be derived from the nucleotide sequences encoding the D2E7 LCVR and HCVR using the genetic code and standard molecular biology techniques.

Recombinant human antibodies of the invention in addition to D2E7 or an antigen binding portion thereof, or D2E7-related antibodies disclosed herein can be isolated by screening of a recombinant combinatorial antibody library, preferably a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SurfZAP™ phage display kit, catalog no. 240612), examples of methods and reagents particularly amenable for use in generating and screening antibody display libraries can be found in, for example, Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-65; Huse et al. (1989) Science 246:1275-1281; McCafferty et al., Nature (1990) 348:552-554; Griffiths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; and Barbas et al. (1991) PNAS 88:7978-7982.

In a preferred embodiment, to isolate human antibodies with high affinity and a low off rate constant for hTNFα, a murine anti-hTNFα antibody having high affinity and a low off rate constant for hTNFα (e.g., MAK 195, the hybridoma for which has deposit number ECACC 87 050801) is first used to select human heavy and light chain sequences having similar binding activity toward hTNFα, using the epitope imprinting methods described in Hoogenboom et al., PCT Publication No. WO 93/06213. The antibody libraries used in this method are preferably scFv libraries prepared and screened as described in McCafferty et al., PCT Publication No. WO 92/01047, McCafferty et al., Nature (1990) 348:552-554; and Griffiths et al., (1993) EMBO J. 12:725-734. The scFv antibody libraries preferably are screened using recombinant human TNFα as the antigen.

Once initial human VL and VH segments are selected, “mix and match” experiments, in which different pairs of the initially selected VL and VH segments are screened for hTNFα binding, are performed to select preferred VL/VH pair combinations. Additionally, to further improve the affinity and/or lower the off rate constant for hTNFα binding, the VL and VH segments of the preferred VL/VH pair(s) can be randomly mutated, preferably within the CDR3 region of VH and/or VL, in a process analogous to the in vivo somatic mutation process responsible for affinity maturation of antibodies during a natural immune response. This in vitro affinity maturation can be accomplished by amplifying VH and VL regions using PCR primers complimentary to the VH CDR3 or VL CDR3, respectively, which primers have been “spiked” with a random mixture of the four nucleotide bases at certain positions such that the resultant PCR products encode VH and VL segments into which random mutations have been introduced into the VH and/or VL CDR3 regions. These randomly mutated VH and VL segments can be rescreened for binding to hTNFα and sequences that exhibit high affinity and a low off rate for hTNFα binding can be selected.

Following screening and isolation of an anti-hTNFα antibody of the invention from a recombinant immunoglobulin display library, nucleic acid encoding the selected antibody can be recovered from the display package (e.g., from the phage genome) and subcloned into other expression vectors by standard recombinant DNA techniques. If desired, the nucleic acid can be further manipulated to create other antibody forms of the invention (e.g., linked to nucleic acid encoding additional immunoglobulin domains, such as additional constant regions). To express a recombinant human antibody isolated by screening of a combinatorial library, the DNA encoding the antibody is cloned into a recombinant expression vector and introduced into a mammalian host cells, as described in further detail in above.

Methods of isolating human neutralizing antibodies with high affinity and a low off rate constant for hTNFα are described in U.S. Pat. Nos. 6,090,382, 6,258,562, and 6,509,015, each of which is incorporated by reference herein.

Antibodies, antibody-portions, and other TNFα inhibitors for use in the methods of the invention, can be incorporated into pharmaceutical compositions suitable for administration to a subject. Typically, the pharmaceutical composition comprises an antibody, antibody portion, or other TNFα inhibitor, and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody, antibody portion, or other TNFα inhibitor.

The compositions for use in the methods and compositions of the invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies or other TNFα inhibitors. The preferred mode of administration is parenteral (e.g., intravenous, subcutaneous, intraperitoneal, intramuscular). In a preferred embodiment, the antibody or other TNFα inhibitor is administered by intravenous infusion or injection. In another preferred embodiment, the antibody or other TNFα inhibitor is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody, antibody portion, or other TNFα inhibitor) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In one embodiment, the invention includes pharmaceutical compositions comprising an effective TNFα inhibitor and a pharmaceutically acceptable carrier, wherein the effective TNFα inhibitor may be used to treat psoriasis. In another embodiment, the invention includes pharmaceutical compositions comprising an effective TNFα inhibitor and a pharmaceutically acceptable carrier, wherein the effective TNFα inhibitor may be used to treat Crohn's disease.

In one embodiment, the antibody or antibody portion for use in the methods of the invention is incorporated into a pharmaceutical formulation as described in PCT/IB03/04502 and U.S. Appln. No. 20040033228 (10/525,292), incorporated by reference herein. This formulation includes a concentration 50 mg/ml of the antibody D2E7 (adalimumab), wherein one pre-filled syringe contains 40 mg of antibody for subcutaneous injection.

In one embodiment, the invention includes a device for delivering a TNFα inhibitor. In one embodiment, the invention includes an automatic injection device as described in PCT/US2007/015095 and U.S. patent application Ser. Nos. 11/824,516 and 12/074,704, incorporated by reference herein.

The antibodies, antibody-portions, and other TNFα inhibitors of the present invention can be administered by a variety of methods known in the art, although for many therapeutic applications, the preferred route/mode of administration is parenteral, e.g., subcutaneous injection. In another embodiment, administration is via intravenous injection or infusion.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, Robinson, ed., Dekker, Inc., New York, 1978.

In one embodiment, the TNFα antibodies and inhibitors used in the invention are delivered to a subject subcutaneously. In one embodiment, the subject administers the TNFα inhibitor, including, but not limited to, TNFα antibody, or antigen-binding portion thereof, to himself/herself.

The TNFα antibodies and inhibitors used in the invention may also be administered in the form of protein crystal formulations which include a combination of protein crystals encapsulated within a polymeric carrier to form coated particles. The coated particles of the protein crystal formulation may have a spherical morphology and be microspheres of up to 500 micro meters in diameter or they may have some other morphology and be microparticulates. The enhanced concentration of protein crystals allows the antibody of the invention to be delivered subcutaneously. In one embodiment, the TNFα antibodies of the invention are delivered via a protein delivery system, wherein one or more of a protein crystal formulation or composition, is administered to a subject with a TNFα-related disorder. Compositions and methods of preparing stabilized formulations of whole antibody crystals or antibody fragment crystals are also described in WO 02/072636, which is incorporated by reference herein. In one embodiment, a formulation comprising the crystallized antibody fragments described in PCT/IB03/04502 and U.S. Appln. No. 20040033228, incorporated by reference herein, are used to treat rheumatoid arthritis using the treatment methods of the invention.

In certain embodiments, an antibody, antibody portion, or other TNFα inhibitor of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, an antibody or antibody portion for use in the methods of the invention is coformulated with and/or coadministered with one or more additional therapeutic agents, including an Psoriasis inhibitor or antagonist. For example, an anti-hTNFα antibody or antibody portion of the invention may be coformulated and/or coadministered with one or more additional antibodies that bind other targets associated with TNFα related disorders (e.g., antibodies that bind other cytokines or that bind cell surface molecules), one or more cytokines, soluble TNFα receptor (see e.g., PCT Publication No. WO 94/06476) and/or one or more chemical agents that inhibit hTNFα production or activity (such as cyclohexane-ylidene derivatives as described in PCT Publication No. WO 93/19751) or any combination thereof. Furthermore, one or more antibodies of the invention may be used in combination with two or more of the foregoing therapeutic agents. Such combination therapies may advantageously utilize lower dosages of the administered therapeutic agents, thus avoiding possible side effects, complications or low level of response by the patient associated with the various monotherapies.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody, antibody portion, or other TNFα inhibitor may vary according to factors such as the disease state, age, sex, and weight of the individual, and the ability of the antibody, antibody portion, other TNFα inhibitor to elicit a desired response in the individual. A therapeutically effective amount is also one in which any toxic or detrimental effects of the antibody, antibody portion, or other TNFα inhibitor are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Additional description regarding methods and uses of the invention comprising administration of a TNFα inhibitor are described in Parts III and IV of this specification.

The invention also pertains to packaged pharmaceutical compositions or kits for administering the anti-TNF antibodies of the invention for the treatment of psoriasis or Crohn's disease. In one embodiment of the invention, the kit comprises a TNFα inhibitor, such as an antibody and instructions for administration of the TNFα inhibitor for treatment of psoriasis or Crohn's disease. The instructions may describe how (e.g., subcutaneously), when (e.g., at week 0, week 1, week 2, week 4, biweekly, etc.), and the different doses (e.g., induction and treatment doses) of TNFα inhibitor that shall be administered to a subject for treatment.

Another aspect of the invention pertains to kits containing a pharmaceutical composition comprising a TNFα inhibitor, such as an antibody, and a pharmaceutically acceptable carrier and one or more pharmaceutical compositions each comprising an additional therapeutic agent useful for treating psoriasis or Crohn's disease, and a pharmaceutically acceptable carrier. Alternatively, the kit comprises a single pharmaceutical composition comprising an anti-TNFα antibody, one or more drugs useful for treating psoriasis or Crohn's disease, and a pharmaceutically acceptable carrier. The instructions may describe how (e.g., subcutaneously), when (e.g., at week 0, week 1, week 2, week 4, biweekly, etc.), and the different doses (e.g., induction and treatment doses of TNFα inhibitor and/or the additional therapeutic agent that shall be administered to a subject for treatment.

The kit may contain instructions for dosing of the pharmaceutical compositions for the treatment of psoriasis or Crohn's disease. Additional description regarding articles of manufacture of the invention are described in subsections III and IV.

The package or kit alternatively can contain the TNFα inhibitor and it can be promoted for use, either within the package or through accompanying information, for the uses or treatment of the disorders described herein. The packaged pharmaceuticals or kits further can include a second agent (as described herein) packaged with or copromoted with instructions for using the second agent with a first agent (as described herein).

### III. Uses and Compositions for Treating Psoriasis

Tumor necrosis factor has been implicated in the pathophysiology of psoriasis (Takematsu et al. (1989) Arch Dermatol Res. 281:398; Victor and Gottlieb (2002) J Drugs Dermatol. 1(3):264). Psoriasis is described as a skin inflammation (irritation and redness) characterized by frequent episodes of redness, itching, and thick, dry, silvery scales on the skin. In particular, lesions are formed which involve primary and secondary alterations in epidermal proliferation, inflammatory responses of the skin, and an expression of regulatory molecules such as lymphokines and inflammatory factors. Psoriatic skin is morphologically characterized by an increased turnover of epidermal cells, thickened epidermis, abnormal keratinization, inflammatory cell infiltrates into the epidermis and polymorphonuclear leukocyte and lymphocyte infiltration into the epidermis layer resulting in an increase in the basal cell cycle. Psoriasis often involves the nails, which frequently exhibit pitting, separation of the nail, thickening, and discoloration. Psoriasis is often associated with other inflammatory disorders, for example arthritis, including rheumatoid arthritis, inflammatory bowel disease (IBD), and to Crohn's disease.

Evidence of psoriasis is most commonly seen on the trunk, elbows, knees, scalp, skin folds, or fingernails, but it may affect any or all parts of the skin. Normally, it takes about a month for new skin cells to move up from the lower layers to the surface. In psoriasis, this process takes only a few days, resulting in a build-up of dead skin cells and formation of thick scales. Symptoms of psoriasis include: skin patches, that are dry or red, covered with silvery scales, raised patches of skin, accompanied by red borders, that may crack and become painful, and that are usually lovated on the elbows, knees, trunk, scalp, and hands; skin lesions, including pustules, cracking of the skin, and skin redness; joint pain or aching which may be associated with of arthritis, e.g., psoriatic arthritis.

Treatment for psoriasis often includes a topical corticosteroids, vitamin D analogs, and topical or oral retinoids, or combinations thereof. In one embodiment, the TNFα inhibitor of the invention is administered in combination with or the presence of one of these common treatments. Additional therapeutic agents which can also be combined with the TNFα inhibitor of the invention for treatment of psoriasis are described in more detail below.

The diagnosis of psoriasis is usually based on the appearance of the skin. Additionally a skin biopsy, or scraping and culture of skin patches may be needed to rule out other skin disorders. An x-ray may be used to check for psoriatic arthritis if joint pain is present and persistent.

In one embodiment of the invention, a TNFα inhibitor is used to treat psoriasis, including chronic plaque psoriasis, guttate psoriasis, inverse psoriasis, pustular psoriasis, pemphigus vulgaris, erythrodermic psoriasis, psoriasis associated with inflammatory bowel disease (IBD), and psoriasis associated with rheumatoid arthritis (RA). Specific types of psoriasis included in the treatment methods of the invention are described in detail below:

a. Chronic Plaque Psoriasis

Tumor necrosis factor has been implicated in the pathophysiology of chronic plaque psoriasis (Asadullah et al. (1999) Br J. Dermatol. 141:94). Chronic plaque psoriasis (also referred to as psoriasis vulgaris) is the most common form of psoriasis. Chronic plaque psoriasis is characterized by raised reddened patches of skin, ranging from coin-sized to much larger. In chronic plaque psoriasis, the plaques may be single or multiple, they may vary in size from a few millimeters to several centimeters. The plaques are usually red with a scaly surface, and reflect light when gently scratched, creating a “silvery” effect. Lesions (which are often symmetrical) from chronic plaque psoriasis occur all over body, but with predilection for extensor surfaces, including the knees, elbows, lumbosacral regions, scalp, and nails. Occasionally chronic plaque psoriasis can occur on the penis, vulva and flexures, but scaling is usually absent. Diagnosis of patients with chronic plaque psoriasis is usually based on the clinical features described above. In particular, the distribution, color and typical silvery scaling of the lesion in chronic plaque psoriasis are characteristic of chronic plaque psoriasis.

b. Guttate Psoriasis

Guttate psoriasis refers to a form of psoriasis with characteristic water drop shaped scaly plaques. Flares of guttate psoriasis generally follow an infection, most notably a streptococcal throat infection. Diagnosis of guttate psoriasis is usually based on the appearance of the skin, and the fact that there is often a history of recent sore throat.

c. Inverse Psoriasis

Inverse psoriasis is a form of psoriasis in which the patient has smooth, usually moist areas of skin that are red and inflammed, which is unlike the scaling associated with plaque psoriasis. Inverse psoriasis is also referred to as intertiginous psoriasis or flexural psoriasis. Inverse psoriasis occurs mostly in the armpits, groin, under the breasts and in other skin folds around the genitals and buttocks, and, as a result of the locations of presentation, rubbing and sweating can irriate the affected areas.

d. Pustular Psoriasis

Pustular psoriasis is a form of psoriasis that causes pus-filled blisters that vary in size and location, but often occur on the hands and feet. The blisters may be localized, or spread over large areas of the body. Pustular psoriasis can be both tender and painful, can cause fevers.

e. Other Psoriasis Disorders

Other examples of psoriatic disorders which can be treated with the TNFα antibody of the invention include erythrodermic psoriasis, vulgaris, psoriasis associated with IBD, and psoriasis associated with arthritis, including rheumatoid arthritis.

TNFα is an important cytokine in the pathogenesis of psoriasis, with elevated concentrations of TNFα playing a role in pathologic inflammation. Psoriasis is a chronic, inflammatory proliferative disease of the skin that affects 1-3% of the general population (Greaves and Weinstein (1995) N Engl J Med 332: 581). Treatment of moderate to severe psoriasis with systemic therapy such as methotrexate or cyclosporine or biologic therapy such as efalizumab can be limited by lack of efficacy or precluded by side effects. Ultraviolet light therapy is often inconvenient. The methods and uses described herein provide a means of determining the efficacy of a TNFα inhibitor for treating psoriasis. In one embodiment, the invention provides a method for treating psoriasis in a subject having psoriasis.

### Clinical Severity of Psoriasis

Severity of psoriasis may be determined according to standard clinical definitions. For example, the Psoriasis Area and Severity Index (PASI) is used by dermatologists to assess psoriasis disease intensity. This index is based on the quantitative assessment of three typical signs of psoriatic lesions: erythema, infiltration, and desquamation, combined with the skin surface area involvement. Since its development in 1978, this instrument has been used throughout the world by clinical investigators (Fredriksson T, Petersson U: Severe psoriasis—oral therapy with a new retinoid. Dermatologica 1978; 157: 238-41). PASI is indicated as PASI 50 (a 50 percent improvement in PASI from baseline), PASI 75 (a 75 percent improvement in PASI from baseline), PASI 90 (a 90 percent improvement in PASI from baseline), and PASI 100 (a 100 percent improvement in PASI from baseline). The efficacy of a TNFα inhibitor for treatment of psoriasis in a patient population who has psoriasis, may be evaluated by determining the percentage of the patient population in whom a PASI 50, PASI 75, PASI 90, or PASI 100 response has been achieved following administration of the TNFα inhibitor.

The Physicians Global Assessment (PGA) is used to assess psoriasis activity and follow clinical response to treatment. It is a six-point score that summarizes the overall quality (erythema, scaling and thickness) and extent of plaques relative to the baseline assessment. A patient's response is rated as worse, poor (0-24%), fair (25-49%), good (50-74%), excellent (75-99%), or cleared (100%) (van der Kerkhof P, Br J Dermatol 1997; 137:661-662). The efficacy of a TNFα inhibitor for psoriasis in a patient may be evaluated by determining the percentage of the patient population in whom an improvement of at least 2 points in the PGA score is achieved following administration of the TNFα inhibitor.

Other measures of improvements in the disease state of a subject having psoriasis include clinical responses, such as the Dermatology Life Quality Index (DLQI) and the Minimum Clinically Important Difference (MCID), described in more detail below.

### Methods of Treatment

Methods of treatment described herein may include administration of a TNFα inhibitor to a subject to achieve a therapeutic goal, e.g., treatment of psoriasis, increase in PASI response, maintenance of a level of PASI response, improvement in PASI score, and/or achievement of a PGA score of “clear” or “almost clear.” Also included in the scope of the invention are uses of a TNFα inhibitor in the manufacture of a medicament to achieve a therapeutic goal, e.g., treatment, of psoriasis, increase in PASI response, maintenance of a level of PASI response, and/or improvement in PASI score, and/or achievement of a PGA score of “clear” or “almost clear.” Thus, where methods are described herein, it is also intended to be part of this invention that the use of the TNFα inhibitor in the manufacture of a medicament for the purpose of the method is also considered within the scope of the invention. Likewise, where a use of a TNFα inhibitor in the manufacture of a medicament for the purpose of achieving a therapeutic goal is described, methods of treatment resulting in the therapeutic goal are also intended to be part of the invention.

The invention also provides pharmacokinetic parameters which have been identified as providing a therapeutic benefit to a subject having psoriasis. Certain mean steady-state trough levels of a TNFα inhibitor have be identified as corresponding to therapeutic benefits for subject having psoriasis, including, but not limited to, treatment of psoriasis. The term “trough level” refers to the serum TNFα inhibitor concentration at a time after delivery of a previous dose and immediately prior to delivery of the next subsequent dose of drug in a series of doses. Generally, the trough serum concentration is a minimum sustained efficacious drug concentration in the series of drug administrations. Also, the trough serum concentration is frequently targeted as a minimum serum concentration for efficacy because it represents the serum concentration at which another dose of drug is to be administered as part of the treatment regimen. In one embodiment, the invention provides a method of treating psoriasis in a subject comprising administering adalimumab, wherein the mean steady-state trough concentration was approximately 5 to 6 μg/mL during adalimumab 40 mg every other week monotherapy treatment. In one embodiment, the invention provides a method of treating of psoriasis in a subject comprising administering a of the TNFα inhibitor, e.g., human TNFα antibody, or antigen-binding portion thereof, to the subject, wherein the maintenance dose provides a mean serum trough level of about 7 μg/mL of the TNFα inhibitor.

In one embodiment, treatment and remission of psoriasis is achieved using multiple variable dosing methods of treatment. Examples of such multiple variable dosing regimens are described in PCT Appln. no. PCT/US05/12007, incorporated by reference herein.

In one embodiment, the invention provides a method of treating psoriasis in a subject in need thereof comprising administering a loading dose of a TNFα inhibitor, e.g., human TNFα antibody, or antigen-binding portion thereof, to the subject, wherein the loading dose provides a mean serum TNFα inhibitor trough level of about 12 μg/mL. Once treatment has been achieved, e.g., PASI 50 or PASI 75 response has been achieved, a maintenance dose(s) of the TNFα inhibitor, e.g., human TNFα antibody, or antigen-binding portion thereof, may be administered to the subject in order to maintain treatment of psoriasis, wherein the maintenance dose provides a mean serum trough level of about 7 μg/mL of the TNFα inhibitor.

In one embodiment, the invention provides a method of treating psoriasis in a subject comprising administering an initial loading dose of a TNFα inhibitor to the subject at week 0. In one embodiment, the initial dose is given in its entirety on one day or is divided over 2 days. In one embodiment, the initial dose is administered subcutaneously. Following administration of the initial loading dose, a second dose of the TNFα inhibitor may be administered to the subject, wherein the second dose is about half the dose amount of the initial loading dose. In one embodiment, the second dose is administered to the subject about two weeks after the first dose. In one embodiment, the second dose is administered to the subject about two weeks after the first dose. In one embodiment, the second dose is administered subcutaneously. Subsequent doses may be administered following the second dose in order to achieve treatment of the subject. Such additional doses may, in one embodiment of the invention, comprise half the dose amount of the second dose or, in another embodiment, comprise the same dose as the second dose. Once treatment is achieved, at least one maintenance dose of the TNFα inhibitor is administered to the subject in order to maintain treatment of psoriasis. In one embodiment, the maintenance dose is about half the dose amount of the second dose or, in another embodiment, comprise the same dose as the second dose. In one embodiment, the maintenance dose is administered to the subject about two weeks after the second dose. In one embodiment, the maintenance therapy for administering the TNFα inhibitor comprises a biweekly dosing regimen. In one embodiment, the maintenance dose is administered subcutaneously.

In one embodiment, the invention provides a method of treating psoriasis in a subject comprising administering an initial loading dose of a human TNFα antibody, or antigen-binding portion thereof, e.g., adalimumab, to the subject at week 0. The initial dose may be given in its entirety on one day or may be divided over 2 days. In one embodiment, the initial dose of the human TNFα antibody, or antigen-binding portion thereof, comprises 80 mg. In one embodiment, the initial dose is administered subcutaneously. Following administration of the initial loading dose, a second dose of the human TNFα antibody, or antigen-binding portion thereof, e.g., adalimumab, is administered to the subject. In one embodiment, the second dose comprises 80 mg of the human TNFα antibody, or antigen-binding portion thereof. In one embodiment, the second dose comprises 40 mg of the human TNFα antibody, or antigen-binding portion thereof. In one embodiment, the second dose is administered to the subject about one week after the first dose. In one embodiment, the second dose is administered subcutaneously. In order to maintain treatment psoriasis once it is achieved, at least one maintenance dose of the human TNFα antibody, or antigen-binding portion thereof, e.g., adalimumab, is administered to the subject. In one embodiment, the maintenance dose is about half the dose amount of the second dose or, in another embodiment, comprise the same dose as the second dose. In one embodiment, the maintenance dose of the human TNFα antibody, or antigen-binding portion thereof, comprises 40 mg. In one embodiment, the maintenance therapy for administering the human TNFα antibody, or antigen-binding portion thereof, comprises a biweekly dosing regimen. In one embodiment, the maintenance dose is administered subcutaneously.

In another embodiment, the initial dose of the human TNFα antibody, or antigen-binding portion thereof, comprises 80 mg and may be given at week 0, followed by at least one maintenance dose of the human TNFα antibody, or antigen-binding portion thereof, comprising 40 mg, administered on a biweekly dosing regimen. In some embodiments, the initial dose of the human TNFα antibody, or antigen-binding portion thereof, comprises 80 mg and may be given at week 0, followed by at least one maintenance dose of the human TNFα antibody, or antigen-binding portion thereof, comprising 40 mg, administered on a biweekly dosing regimen, starting given one week after the 80 mg dose. In some embodiments, the initial dose of 80 mg is followed by a maintenance dose of 40 mg one week later, followed by an additional maintenance dose of 40 mg, two weeks after the first maintenance dose of 40 mg. In some embodiments, the second maintenance dose of 40 mg is followed by additional maintenance doses of 40 mg each, every two weeks.

In one embodiment, maintenance of remission of psoriasis is achieved by administering a TNFα inhibitor to a subject in accordance with a biweekly dosing regimen. Biweekly dosing regimens can be used to treat disorders in which TNFα activity is detrimental, and are further described in U.S. application Ser. No. 10/163,657 (US 20030235585), incorporated by reference herein. In one embodiment, treatment of psoriasis is achieved by administering a human TNFα antibody, or an antigen-binding portion thereof, to a subject having psoriasis, wherein the human TNFα antibody, or an antigen-binding portion thereof, is administered on a biweekly dosing regimen. In one embodiment, biweekly dosing includes a dosing regimen wherein doses of a TNFα inhibitor are administered to a subject every other week consecutively for a given time period, e.g., 4 weeks, 8 weeks, 16, weeks, 24 weeks, 26 weeks, 32 weeks, 36 weeks, 42 weeks, 48 weeks, 52 weeks, 56 weeks, etc. Biweekly dosing is preferably administered parenterally, including subcutaneously. In one embodiment, the human TNFα antibody, or an antigen-binding portion thereof, is administered in a dose of about 40 mg.

Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

Dosage regimens described herein may be adjusted to provide the optimum desired response, e.g., maintaining remission of psoriasis, in consideration of the teachings herein. It is to be noted that dosage values may vary with the type and severity of psoriasis. It is to be further understood that for any particular subject, specific dosage regimens may be adjusted over time according to the teachings of the specification and the individual need and the professional judgment of the person administering or supervising the administration of the compositions, and that dosage amounts and ranges set forth herein are exemplary only and are not intended to limit the scope or practice of the claimed invention.

The invention also provides a method of treating psoriasis-related disorders, comprising administering a TNFα inhibitor to a subject. The TNFα inhibitors used in the present invention may be administered by a variety of methods known in the art, although for many therapeutic applications, the preferred route/mode of administration is parenteral, including intravenous or subcutaneous injection. Examples of other methods and uses of TNFα inhibitors for the treatment of psoriasis are also described in U.S. patent application Ser. No. 11/880,433; PCT/US2007/009131; U.S. patent application Ser. No. 11/811,141; and U.S. patent application Ser. No. 11/800,531, incorporated by reference herein.

### Subpopulations

The invention provides uses and methods for treating certain subpopulations of psoriasis patients with a TNFα inhibitor.

In one embodiment, the invention provides a method of treating moderate to severe psoriasis in a subject comprising administering to the subject a TNFα inhibitor, such that moderate to severe psoriasis is treated. Subjects having moderate to severe psoriasis may be administered a TNFα inhibitor such that moderate to severe psoriasis is treated and advancement of the disease is prevented. The invention also provides use of a TNFα inhibitor in the manufacture of a medicament for the treatment of moderate to severe psoriasis in a subject who has moderate to severe psoriasis. In a preferred to embodiment, a patient having moderate to severe psoriasis is defined as a patient having a PASI<10. In another embodiment, a patient having moderate to severe psoriasis is defined as a patient having a PASI<15. In another embodiment, a patient having moderate to severe psoriasis is defined as a patient having a PASI<20. In another embodiment, a patient having moderate to severe psoriasis is defined as a patient having a PASI<25. In another embodiment, a patient having moderate to severe psoriasis is defined as a patient having a PASI<30. In another embodiment, a patient having moderate to severe psoriasis is defined as a patient having a PASI<35. In another embodiment, a patient having moderate to severe psoriasis is defined as a patient having a PASI<40.

Numbers below and intermediate to the above recited PASI responses, e.g., 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, and 39%, as well as all other numbers recited herein, are also intended to be part of this invention. Ranges of values using a combination of any of the above recited values as upper and/or lower limits are intended to be included in the scope of the invention. For example, in one embodiment a PASI 90 response score of in at least between 0% and 40% of the patient population indicates that the patient has moderate to severe psoriasis.

The invention also provides a method for treating a subpopulation of psoriasis patients who are intolerant to, who have had a subtherapeutic response to, or who are not clinical candidates for a psoriasis therapy, e.g., phototherapy.

The invention also provides a method for treating a subpopulation of psoriasis patients who are intolerant to or have lost response to a first TNFα inhibitor, e.g., infliximab, for the treatment of psoriasis. Clinical trials have demonstrated the efficacy of infliximab, a chimeric monoclonal antibody to TNF, for treatment of patients with moderate to severe psoriasis. Infusions of infliximab, especially when given episodically, may result in the development of antibodies to infliximab, however, which in turn may lead to infusion reactions, loss of efficacy, and delayed hypersensitivity reactions (Baert et al. N Engl J Med 2003; 348:601-608; Cheifetz et al. Am J Gastroenterol 2003; 98:1315-1324; Farrell et al. Gastroenterology 2003; 124:917-924; Hanauer et al. Gastroenterology 1999; 116:A731; and Hanauer et al. Clin Gastroenterol Hepatol 2004; 2:542-553). In certain instances, some patients who are administered a TNFα inhibitor for the treatment of psoriasis and respond to said treatment, may eventually lose their response to the first TNFα inhibitor. In other patient populations, intolerance to a certain TNFα inhibitor may be marked from the initial administration of the TNFα inhibitor. In one embodiment, the invention provides use of a TNFα inhibitor in the manufacture of a medicament for treating psoriasis in a subject who has lost response to or is intolerant to a different TNFα inhibitor. In one embodiment, the TNFα inhibitor which the subject has lost response to or is intolerant to is infliximab.

In one embodiment, the invention also provides methods and compositions for use in a subject who has not previously been administered infliximab. Thus, in one embodiment, the methods and compositions of the invention are directed to a subpopulation of psoriasis patients who have not previously received infliximab.

In one embodiment, the invention provides an article of manufacture comprising adalimumab and a package insert, wherein the package insert indicates that adalimumab may be used to treat psoriasis in patients who have had an inadequate response to conventional therapy and/or who have lost response to or are intolerant to infliximab.

### Articles of Manufacture

The invention also provides a packaged pharmaceutical composition wherein the TNFα□ inhibitor, e.g., human TNFα□ antibody, is packaged within a kit or an article of manufacture. The kit or article of manufacture of the invention contains materials useful for the treatment, including induction and/or remission, prevention and/or diagnosis of psoriasis. The kit or article of manufacture comprises a container and a label or package insert or printed material on or associated with the container which provides information regarding use of the TNFα inhibitor, e.g., a TNFα antibody, for the treatment of psoriasis.

A kit or an article of manufacture refers to a packaged product comprising components with which to administer a TNFα inhibitor for treatment of a psoriasis. The kit preferably comprises a box or container that holds the components of the kit. The box or container is affixed with a label or a Food and Drug Administration approved label, including a protocol for administering the TNFα inhibitor. The box or container holds components of the invention which are preferably contained within plastic, polyethylene, polypropylene, ethylene, or propylene vessels. The vessels can be capped-tubes or bottles. The kit can also include instructions for administering the TNFα antibody of the invention. In one embodiment the kit of the invention includes the formulation comprising the human antibody adalimumab (or D2E7), as described in PCT/IB03/04502 and U.S. application Ser. No. 10/222,140, incorporated by reference herein.

The term “package insert” or “label” is used to refer to instructions customarily included in commercial packages of therapeutic products, that contain information about the indications, usage, dosage, administration, contraindications and/or warnings concerning the use of such therapeutic products.

In one embodiment, the article of manufacture of the invention comprises (a) a first container with a composition contained therein, wherein the composition comprises a TNFα antibody; and (b) a package insert indicating that the TNFα antibody may be used to for reducing signs and symptoms and inducing and maintaining remission of psoriasis. In a preferred embodiment, the label or package insert indicates that the TNFα inhibitor, e.g., a TNFα antibody, is used for treating psoriasis, e.g, plaque psoriasis.

In one embodiment, the label or package insert indicates that the package contains suitable containers for the TNFα inhibitor (e.g., a TNFα antibody), for example, bottles, vials, syringes, pens, etc. The containers may be formed from a variety of materials such as glass or plastic. The container holds a composition which is by itself or when combined with another composition effective for treating, preventing and/or diagnosing the condition and may have a sterile access port. In one embodiment, the label or package insert indicates that the TNFα antibody, e.g., adalimumab, is provided as one or more 40 mg/0.8 mL prefilled single-use pen(s). In another embodiment, the label or package insert indicates that the TNFα antibody, e.g., adalimumab, is provided in one or more 40 mg/0.8 mL single-use prefilled glass syringe(s).

In one embodiment, the article of manufacture comprises a TNFα inhibitor, e.g., a human TNFα antibody, and a label or package insert which indicates to a subject who will be administering the TNFα inhibitor about using the TNFα inhibitor for the treatment of psoriasis. The label may be anywhere within or on the article of manufacture. In one embodiment, the article of manufacture comprises a container, such as a box, which comprises the TNFα inhibitor and a package insert or label providing information pertaining to use of the TNFα inhibitor for the treatment of psoriasis. In another embodiment, the information is printed on a label which is on the outside of the article of manufacture, in a position which is visible to a reader, e.g., a prospective purchaser.

In one embodiment, the label or package insert of the invention informs a reader, including a subject, e.g., a purchaser, who will be administering the TNFα inhibitor for treatment, that the TNFα inhibitor, e.g., a TNFα antibody such as adalimumab, is an indicated treatment of psoriasis, including of moderately to severely active disease in adult patients.

In one embodiment, the label or package insert describes certain patient populations who may respond favorably to the TNFα inhibitor within the article of manufacture. For example, the label or package insert may indicate that the TNFα antibody, e.g., adalimumab, may be used to treat psoriasis in patients who have had an inadequate response to conventional therapy and/or who have lost response to or are intolerant to infliximab. The label or package insert may indicate that the TNFα antibody, e.g., adalimumab, may be used for the treatment of adult patients with moderate to severe chronic plaque psoriasis who are candidates for systemic therapy or phototherapy, and when other systemic therapies are medically less appropriate. The package insert may also indicate that adalimumab should only be administered to patients who will be closely monitored and have regular follow-up visits with a physician. In one embodiment, the package insert of the invention indicates that adalimumab may be used to treat moderate to severe chronic (lasting a long time) plaque psoriasis (Ps) in adults who have the condition in many areas of their body and who may benefit from taking injections or pills (systemic therapy) or phototherapy (treatment using ultraviolet light alone or with pills).

In another embodiment, the label of the invention indicates that adalimumab is indicated for treatment of moderately to severely active psoriasis in adult patients who have had an inadequate response to conventional therapy. In another embodiment, the label of the invention indicates that the TNFα inhibitor, e.g., a TNFα antibody such as adalimumab, is also indicated for treatment in adult patients with moderately to severely active psoriasis who have lost response to or are intolerant to infliximab.

In one embodiment, the package insert of the invention describes certain therapeutic benefits of the TNFα antibody, e.g., adalimumab, including specific symptoms of psoriasis which may be reduced by using the TNFα antibody, e.g., adalimumab. It should be noted that the package insert may also contain information pertaining to other disorders which are treatable using the TNFα antibody, e.g., adalimumab. Information described herein which is provided in a package insert and pertains to other disorders, i.e., diseases other than psoriasis, is also included within the scope of the invention. The package insert of the invention may indicate that extra TNFα in your body can attack normal healthy body tissues and cause inflammation especially in the tissues in your bones, cartilage, joints and digestive tract. The package insert of the invention may also indicate that adalimumab helps reduce the signs and symptoms of immune diseases, including rheumatoid and psoriatic arthritis (pain and swollen joints), ankylosing spondylitis (morning stiffness and back pain), and psoriasis (abdominal pain and diarrhea).

In another embodiment, the label or package insert of the invention describes the dose and administration of adalimumab, for the treatment of psoriasis. The label may indicate that the initiation of therapy includes an initial dose of 80 mg dose at week 0 and 80 mg at week 1. The label may also indicate that the maintenance dosing for the treatment of psoriasis with adalimumab is 40 mg every other week. In one embodiment, the package insert indicates that the week 0 dose may be administered as 4 injections in one day or divided over 2 days. In one embodiment, the package insert indicates that the week 0 dose may be administered as 2 injections in one day or divided over 2 days. The label may also indicate that some patients with psoriasis may derive additional benefit by increasing frequency to 40 mg every week. In another embodiment, the package insert of the invention indicates that adalimumab is administered by subcutaneous injection.

In another embodiment, the label of the invention indicates that the recommended TNFα inhibitor, e.g., a TNFα antibody such as adalimumab, dose regimen for adult patients with psoriasis is 80 mg at week 0 (dose can be administered as four injections in one day or as two injections per day for two consecutive days), followed by 40 mg every other week beginning at week 2. In another embodiment the label indicates that the recommended TNFα inhibitor, e.g., a TNFα antibody such as adalimumab, dose regimen for adult patients with psoriasis is 80 mg at week 0, followed by 40 mg every other week beginning at week 1 The label of the invention may also indicate that some patients may derive additional benefit from increasing the dosing frequency of the TNFα inhibitor, e.g., a TNFα antibody such as adalimumab from 40 mg every other week to 40 mg every week.

In another embodiment, the label of the invention indicates that the recommended TNFα inhibitor, e.g., a TNFα antibody such as adalimumab, dose regimen for adult patients with psoriasis is 40 mg at week 0 (dose can be administered as four injections in one day or as two injections per day for two consecutive days), followed by 40 mg every other week beginning at week 2. The label of the invention may also indicate that some patients may derive additional benefit from increasing the dosing frequency of the TNFα inhibitor, e.g., a TNFα antibody such as adalimumab from 40 mg every other week to 40 mg every week.

The invention also includes a package insert which indicates treatment regimens for diseases other than psoriasis. For example, the package insert may indicate that the recommended adalimumab dose regimen for adult patients with Crohn's disease is 160 mg initially at Day 1 (given as four 40 mg injections in one day or as two 40 mg injections per day for two consecutive days), followed by 80 mg two weeks later (Day 15). Two weeks later (Day 29) begin a maintenance dose of 40 mg every other week. The package insert may also indicate that aminosalicylates, corticosteroids, and/or immunomodulatory agents (e.g., 6-mercaptopurine and azathioprine) may be continued during treatment with adalimumab. The package insert may also indicate that the use of adalimumab in Crohn's disease beyond one year has not been evaluated in controlled clinical studies.

The label or package insert of the invention may also provide information to subjects who will be receiving adalimumab regarding combination uses for both safety and efficacy purposes. In another embodiment, the label of the invention indicates that aminosalicylates, corticosteroids, and/or immunomodulatory agents (e.g., 6-mercaptopurine and azathioprine) may be continued during treatment with the TNFα inhibitor, e.g., a TNFα antibody, including adalimumab. In one embodiment, the invention provides an article of manufacture comprising a packaging material; a TNFα antibody, or antigen-binding portion thereof; and a label or package insert contained within the packaging material indicating that aminosalicylates, corticosteroids, and/or immunomodulatory agent, e.g., 6-mercaptopurine and azathioprine, may be continued during treatment with the TNFα antibody, or antigen-binding portion thereof.

The label or package insert of the invention may contain warnings and precautions regarding the use of the TNFα inhibitor, e.g., a TNFα antibody such as adalimumab. In one embodiment, the label cautions against starting treatment during an active infection. In one embodiment, the label indicates that infections that develop during treatment should be monitored and may recommend cessation of treatment if infection becomes serious. In another embodiment, the package insert describes safety for using adalimumab and concurrent vaccinations.

In one embodiment, the information provided in the label describes malignancies. The label of the invention may indicate that during the controlled portions of TNFα antibody, such as adalimumab, trials in patients with rheumatoid arthritis, psoriatic arthritis, ankylosing spondylitis, Crohn's disease, and psoriasis, malignancies, other than lymphoma and non-melanoma skin cancer, were observed at a rate (95% confidence interval) of 0.6 (0.3, 1.0)/100 patient-years among 2887 adalimumab-treated patients versus a rate of 0.4 (0.2, 1.1)/100 patient-years among 1570 control patients (median duration of treatment of 5.7 months for adalimumab-treated patients and 5.5 months for control-treated patients). The label may also indicate that the size of the control group and limited duration of the controlled portions of studies precludes the ability to draw firm conclusions. In one embodiment, the label indicates that in the controlled and uncontrolled open-label portions of the clinical trials of adalimumab, the more frequently observed malignancies, other than lymphoma and non-melanoma skin cancer, were breast, colon, prostate, lung and melanoma. In one embodiment, the label indicates that these malignancies in adalimumab treated and control-treated patients were similar in type and number to what would be expected in the general population. The label may further indicate that during the controlled portions of adalimumab rheumatoid arthritis, psoriatic arthritis, ankylosing spondylitis, Crohn's disease, and psoriasis trials, the rate (95% confidence interval) of non-melanoma skin cancers was 0.8 (0.47, 1.24)/100 patient-years among adalimumab-treated patients 0.2 (0.05, 0.82)/100 patient-years among control patients. In one embodiment, the label indicates that the potential role of TNF blocking therapy in the development of malignancies is not known. In one embodiment, the label indicates that in the controlled portions of clinical trials of all the TNF-blocking agents, more cases of lymphoma have been observed among patients receiving TNF blockers compared to control patients. In one embodiment, the label indicates that in controlled trials in patients with rheumatoid arthritis, psoriatic arthritis, ankylosing spondylitis, and psoriasis, 2 lymphomas were observed among 2887 HUMIRA®-treated patients versus 1 among 1570 control patients. In one embodiment, the label of the invention indicates to that in combining the controlled and uncontrolled open-label portions of these clinical trials with a median duration of approximately 2 years, including 4843 patients and over 13,000 patient-years of therapy, the observed rate of lymphomas is approximately 0.12/100 patient-years, and that this is approximately 3.5-fold higher than expected in the general population.

In one embodiment, the label or package insert indicates that serious allergic reactions may occur. In one embodiment, the label indicates that Hepatitis B virus reactivation may occur. In other embodiments, the package insert or label may indicate that exacerbation of or new onset of demyelinating disease may occur; symptoms of cytopenias or pancytopenia may develop; worsening or new onset of heart failure may occur; lupus-like syndrome may develop; and/or tuberculosis reactivation may occur.

In one embodiment, the label or the package insert of the invention may contain warnings regarding adverse reactions that are specific for psoriasis. In one embodiment, the label or package insert may indicate that common adverse reactions include infections (e.g., upper respiratory, sinusitis), injection site reactions, headache and rash. In another embodiment, the package insert may indicate that clinical trials have shown that adalimumab-treated patients had a higher incidence of arthralgia when compared to controls. In another embodiment, the package insert may indicate that certain aminotransferases, e.g., ALT, AST, were elevated in certain patients who were administered adalimumab.

The label of the invention may contain information regarding the use of the TNFα inhibitor, e.g., a TNFα antibody such as adalimumab, in clinical studies for psoriasis. In one embodiment, the label of the invention describes the studies described herein as Examples, either as a whole or in portion. The label of the invention may also indicate that adalimumab has been studied in over 1200 patients with psoriasis in placebo-controlled and open-label extension studies. The label of the invention may also indicate that the safety profile for patients with psoriasis treated with HUMIRA® was similar to the safety profile seen in patients with rheumatoid arthritis.

The label of the invention may contain information regarding the pharmacodynamics of the TNFα inhibitor, e.g., a TNFα antibody such as adalimumab. In one embodiment, the label of the invention indicates that after treatment with adalimumab, a rapid decrease in levels of acute phase reactants of inflammation (C-reactive protein (CRP) and erythrocyte sedimentation rate (ESR) and serum cytokines (IL-6) was observed compared to baseline in patients with rheumatoid arthritis. In one embodiment, the label of the invention indicates that a rapid decrease in CRP levels was also observed in patients with psoriasis. The label may further indicate that serum levels of matrix metalloproteinases (MMP-1 and MMP-3) that produce tissue remodeling responsible for cartilage destruction were also decreased after adalimumab administration.

The label of the invention may also contain information regarding the pharmacokinetics of the TNFα inhibitor, e.g., a TNFα antibody such as adalimumab. In one embodiment, the label of the invention indicates that in patients with psoriasis, the loading dose of 80 mg adalimumab on week 0 followed by 80 mg adalimumab on week 1 achieves serum adalimumab trough concentrations of approximately 12 μg/mL. The label of the invention may also indicate that mean steady-state trough levels of approximately 7 μg/mL were observed in psoriasis patients who received a maintenance dose of 40 mg adalimumab every other week

In one embodiment, the invention provides an article of manufacture comprising a TNFα inhibitor and a package insert, wherein the package insert indicates that in patients with psoriasis who have been administered the TNFα inhibitor, the loading dose on week 0 followed by a second dose on week 2 achieves serum adalimumab trough concentrations of approximately 12 μg/mL.

In one embodiment, an article of manufacture comprising a TNFα inhibitor and a package insert, wherein the package insert indicates that in patients with psoriasis who have been administered the TNFα inhibitor, the mean steady-state trough levels of approximately 7 μg/mL were observed in psoriasis patients who received a maintenance dose of the TNFα inhibitor every other week.

The label of the invention may also contain information regarding drug interactions of the TNFα inhibitor, e.g., a TNFα antibody such as adalimumab, with other drugs. In one embodiment, the label indicates that methotrexate (MTX) reduced adalimumab apparent clearance after single and multiple dosing by 29% and 44% respectively, in patients with rheumatoid arthritis. In another embodiment, the label may contain information regarding drug interaction with Anakinra (e.g., increased risk of infection). In another embodiment, the label may include a warning against administration of live vaccines during treatment with a TNFα inhibitor, e.g. adalilmumab.

In one embodiment of the invention, the kit comprises a TNFα inhibitor, such as an antibody, a second pharmaceutical composition comprising an additional therapeutic agent, and instructions for administration of both agents for the treatment of psoriasis. The instructions may describe how, e.g., subcutaneously, and when, e.g., at week 0, week 2, and biweekly thereafter, doses of TNFα antibody and/or the additional therapeutic agent shall be administered to a subject for treatment.

Another aspect of the invention pertains to kits containing a pharmaceutical composition comprising an anti-TNFα antibody and a pharmaceutically acceptable carrier and one or more additional pharmaceutical compositions each comprising a drug to useful for treating a TNFα related disorder and a pharmaceutically acceptable carrier. Alternatively, the kit comprises a single pharmaceutical composition comprising an anti-TNFα antibody, one or more drugs useful for treating a TNFα related disorder and a pharmaceutically acceptable carrier. The kits further contain instructions for dosing of the pharmaceutical compositions for the treatment of a TNFα related disorder.

The package or kit alternatively may contain the TNFα inhibitor and it may be promoted for use, either within the package or through accompanying information, for the uses or treatment of the disorders described herein. The packaged pharmaceuticals or kits further can include a second agent (as described herein) packaged with or copromoted with instructions for using the second agent with a first agent (as described herein).

### IV. Uses and Compositions for Treating Crohn's Disease

TNFα is an important cytokine in the pathogenesis of Crohn's disease (CD), with elevated concentrations of TNFα playing a role in pathologic inflammation.

One challenge in the treatment of Crohn's disease is maintaining remission in a subject once remission is achieved. One of the goals in treating Crohn's disease is to control active disease, e.g., to induce and maintain remission in patients/subjects. The methods and uses described herein provide a means of not only inducing remission, but also maintaining remission of Crohn's disease. In one embodiment, the invention provides a method for maintaining remission of Crohn's disease in a subject having Crohn's disease.

Remission of Crohn's disease may be determined according to standard clinical definitions. For example, clinical remission of Crohn's may be defined as a Crohn's Disease Activity Index (CDAI) score of less than 150. The CDAI score is derived as a weighted sum of eight different Crohn's disease-related subjective and objective assessments: extra-intestinal manifestations of Crohn's disease, abdominal mass, use of antidiarrheal drugs, body weight, hematocrit, total number of liquid or soft stools, abdominal pain/cramps, and general well-being. In one embodiment, remission of Crohn's disease is defined as a CDAI of <150. Other measures of improvements in the disease state of a subject having Crohn's disease include clinical responses, such as a decrease in the CDAI score of at least about 70 points or a decrease in the CDAI score of at least about 100.

In one embodiment, the invention provides a method for maintaining remission of Crohn's disease in a subject who has achieved remission of Crohn's disease comprising administering a human TNFα antibody, or an antigen-binding portion thereof, to the subject, such that remission of Crohn's disease is maintained. In one embodiment, the invention describes a use of a human TNFα antibody, or antigen-binding portion thereof, in the manufacture of a medicament for maintaining remission of Crohn's disease in a subject who has achieved remission. The medicament may be for administration to the subject on a maintenance dose regimen. In one embodiment, remission of Crohn's disease is determined by a Crohn's Disease Activity Index (CDAI) score of less than 150 being maintained in the subject. The TNFα antibody, or an antigen-binding portion thereof, may be administered to the subject on a maintenance dose regimen. In one embodiment, a human TNFα antibody, or an antigen-binding portion thereof, is administered to a subject to induce and maintain remission of Crohn's disease.

In clinical trials, the clinical activity of Crohn's disease has traditionally been assessed with the Crohn's Disease Activity Index (CDAI). However, the CDAI does not completely capture the burden of disease on the lives of patients. Therefore, assessment of HRQOL in clinical trials provides valuable information on the comprehensive benefits of treatment, which is complementary to assessment of disease activity. Previous research has examined the benefits of tumor necrosis factor (TNF) antagonists in improving disease-specific HRQOL measures, such as the Inflammatory Bowel Disease Questionnaire (IBDQ) (Guyatt et al. Gastroenterology. 1989; 96:804-10, the contents of which are expressly incorporated herein by reference), and in improving general HRQOL, as measured by the Medical Outcomes Study 36-item Short Form Health Survey (SF-36) (Ware J E, Sherbourne C D. Med Care 1992; 30:473-83; Feagan et al. Am J Gastroenterol 2003; 98:2232-8). The invention also provides a method of determining the efficacy of a TNFα inhibitor for treating Crohn's disease using an Inflammatory Bowel Disease Questionnaire (IBDQ) score of a patient population having Crohn's disease. The IBDQ, a 32-item questionnaire, was developed to provide a measure of health status for clinical trials in inflammatory bowel disease. It evaluates quality of life with respect to bowel function (e.g. loose stools and abdominal pain), systemic symptoms (fatigue and altered sleep pattern), social function (work attendance and the need to cancel social events) and emotional status (angry, depressed, or irritable). The score ranges from 32 to 224, with higher scores indicating a better quality of life. In one embodiment, the methods further comprises administering the effective TNFα inhibitor to a subject having Crohn's disease. However, some important aspects of the burden of Crohn's disease, such as symptoms of depression and fatigue, have rarely been evaluated via depression- or fatigue-specific instruments. Thus, the invention provides a means for treating depression in patients having Crohn's disease, as well as improving fatigue in said patients.

Methods of treatment described herein may include administration of a TNFα inhibitor to a subject to achieve a therapeutic goal, e.g., induction and/or remission of Crohn's disease, decrease in CDAI score, maintenance of a level of CDAI score, and/or improvement in IBDQ score. Also included in the scope of the invention are uses of a TNFα inhibitor in the manufacture of a medicament to achieve a therapeutic goal, e.g., induction and/or remission, of Crohn's disease, decrease in CDAI score, maintenance of a level of CDAI score, and/or improvement in IBDQ score. Thus, where methods are described herein, it is also intended to be part of this invention that the use of the TNFα inhibitor in the manufacture of a medicament for the purpose of the method is also considered within the scope of the invention. Likewise, where a use of a TNFα inhibitor in the manufacture of a medicament for the purpose of achieving a therapeutic goal is described, methods of treatment resulting in the therapeutic goal are also intended to be part of the invention.

In one embodiment, the method and use of a human TNFα antibody, or an antigen-binding portion thereof, for maintaining remission of Crohn's disease, further comprises a method of decreasing steroid use in the subject. While steroids may work effectively, steroids are not effective in preventing flare-ups and thus are rarely used as a maintenance medication in Crohn's disease. Steroids also have many potentially serious side effects—such as elevated blood sugar, high blood pressure, cataracts, osteoporosis (even leading to bone fractures), among others. The risk of adverse effects increases with the duration of the treatment using steroids. The invention provides a means for phasing steroid use out while using a TNFα inhibitor to maintain remission of Crohn's disease. In one embodiment, use of the TNFα inhibitor, including a TNFα antibody, or an antigen-binding portion thereof, results in maintaining remission of Crohn's disease and decreasing steroid use in the subject. In another embodiment, steroid use by a subject having Crohn's is diminished by administering a TNFα inhibitor, e.g., a human TNFα antibody, or an antigen-binding portion thereof, to the subject.

The invention also provides a method of treating Crohn's-related disorders, comprising administering a TNFα inhibitor to a subject. The TNFα inhibitors used in the present invention may be administered by a variety of methods known in the art, although for many therapeutic applications, the preferred route/mode of administration is parenteral, including intravenous or subcutaneous injection. In one embodiment, the invention provides a method of treating fistulas associated with Crohn's disease.

In one embodiment, induction and remission of Crohn's disease is achieved using multiple variable dosing methods of treatment. Examples of such multiple variable dosing regimens are described in PCT Appln. no. PCT/US05/12007, incorporated by reference herein.

In one embodiment, the invention provides a method of inducing and maintaining remission of Crohn's disease in a subject comprising administering an initial loading dose of a human TNFα antibody, or antigen-binding portion thereof, e.g., to adalimumab, to the subject at week 0. In some embodiments, the initial dose may be given in its entirety on one day or may be divided over 2 days. In one embodiment, the initial dose of the human TNFα antibody, or antigen-binding portion thereof, comprises 160 mg. In some embodiments, the initial dose comprises 160 mg and is administered as four 40 mg injections in one day or as two 40 mg injections per day for two consecutive days. In one embodiment, the initial dose is administered subcutaneously. Following administration of the initial loading dose, a second dose of the human TNFα antibody, or antigen-binding portion thereof, e.g., adalimumab, is administered to the subject, wherein the second dose is about half the dose amount of the loading dose. In one embodiment, the second dose comprises 80 mg of the human TNFα antibody, or antigen-binding portion thereof. In one embodiment, the second dose is administered to the subject about two weeks after the first dose. In one embodiment, the second dose is administered subcutaneously. In some embodiments, in order to maintain remission of Crohn's disease once it is achieved, at least one maintenance dose of the human TNFα antibody, or antigen-binding portion thereof, e.g., adalimumab, is administered to the subject. In one embodiment, the maintenance dose is about half the dose amount of the second dose. In one embodiment, the maintenance dose of the human TNFα antibody, or antigen-binding portion thereof, comprises 40 mg. In one embodiment, the maintenance dose is administered to the subject about two weeks after the second dose. In one embodiment, the maintenance therapy for administering the human TNFα antibody, or antigen-binding portion thereof, comprises a biweekly dosing regimen. In one embodiment, the maintenance dose is administered subcutaneously.

In one embodiment, maintenance of remission of Crohn's disease is achieved by administering a human TNFα antibody, or an antigen-binding portion thereof, to a subject having Crohn's disease, wherein the human TNFα antibody, or an antigen-binding portion thereof, is administered on a maintenance therapy comprising a biweekly dosing regimen. Biweekly dosing regimens can be used to treat disorders in which TNFα activity is detrimental, and are further described in U.S. application Ser. No. 10/163,657 (US 20030235585), incorporated by reference herein. In one embodiment, the human TNFα antibody, or an antigen-binding portion thereof, is administered in a dose of about 40 mg. In one embodiment, the human TNFα antibody, or an antigen-binding portion thereof, is adalimumab.

In one embodiment, the invention provides a method for decreasing the risk of hospitalization which is associated with Crohn's disease. By administering a TNFα inhibitor, such as a human TNFα antibody, or an antigen-binding portion thereof, a subject having Crohn's disease may decrease the likelihood that hospitalization will be required. In one embodiment, a TNFα inhibitor, such as a human TNFα antibody, or an antigen-binding portion thereof, is administered as a maintenance therapy to a subject to having Crohn's disease such that the risk of hospitalization is decreased. Decreasing the hospitalization risk of a subject also decreases the cost which is associated with Crohn's disease.

Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

Dosage regimens described herein may be adjusted to provide the optimum desired response, e.g., maintaining remission of Crohn's disease, in consideration of the teachings herein. It is to be noted that dosage values may vary with the type and severity of Crohn's disease. It is to be further understood that for any particular subject, specific dosage regimens may be adjusted over time according to the teachings of the specification and the individual need and the professional judgment of the person administering or supervising the administration of the compositions, and that dosage amounts and ranges set forth herein are exemplary only and are not intended to limit the scope or practice of the claimed invention. Examples of other methods and uses of TNFα inhibitors for the treatment of Crohn's disease are also described in U.S. patent application Ser. No. 11/800,531; U.S. patent application Ser. No. 11/804,725; PCT/US2007/008962; and U.S. patent application Ser. No. 12/008,064, all of which are hereby incorporated in their entirety.

Crohn's-Related Disorders

In addition, the invention provides methods and compositions for treating disorders often associated with Crohn's disease, i.e., Crohn's-related disorders. The term “Crohn's disease-related disorder,” is used to describe disorders and complications associated with Crohn's disease. Examples of Crohn's-related disorders include fistulas in the bladder, vagina, and skin; bowel obstructions; abscesses; nutritional deficiencies; complications from corticosteroid use; inflammation of the joints; erythem nodosum; pyoderma gangrenosum; and lesions of the eye. Other disorders commonly associated with Crohn's disease include Crohn's-related arthralgias, fistulizing Crohn's, indeterminant colitis, and pouchitis.

In one embodiment, the invention provides a method of maintaining remission of a Crohn's-related fistula in a subject comprising administering a TNFα inhibitor to the subject, such that remission of the Crohn's-related fistula is maintained. In one embodiment, the invention provides use of a TNFα inhibitor in the manufacture of a medicament for maintaining remission of a Crohn's-related fistula in a subject.

### Subpopulations

The invention provides uses and methods for treating certain subpopulations of Crohn's patients with a TNFα inhibitor.

In one embodiment, the invention provides a method of treating early Crohn's disease in a subject comprising administering to the subject a TNFα inhibitor, such that early Crohn's disease is treated. Subjects having early Crohn's disease may be administered a TNFα inhibitor such that early Crohn's disease is treated and advancement of the disease is prevented. The invention also provides use of a TNFα inhibitor in the manufacture of a medicament for the treatment of early Crohn's disease in a subject who has early Crohn's disease. In one embodiment, early Crohn's is defined as a disease duration of less than 2 years.

The invention also provides a method for treating a subpopulation of Crohn's patients who are intolerant to or have lost response to a first TNFα inhibitor, e.g., infliximab, for the treatment of Crohn's. Clinical trials have demonstrated the efficacy of infliximab, a chimeric monoclonal antibody to TNF, for induction and maintenance therapy of patients with moderate to severe CD, including those with draining fistulas (Hanauer et al. Lancet 2002; 359:1541-1549; Present et al. N Engl J Med 1999; 340:1398-1405; Rutgeerts et al. Gastroenterology 1999; 117:761-769; Sands et al. N Engl J Med 2004; 350:876-885; and Targan et al. N Engl J Med 1997; 337:1029-1035). Infusions of infliximab, especially when given episodically, may result in the development of antibodies to infliximab, however, which in turn may lead to infusion reactions, loss of efficacy, and delayed hypersensitivity reactions (Baert et al. N Engl J Med 2003; 348:601-608; Cheifetz et al. Am J Gastroenterol 2003; 98:1315-1324; Farrell et al. Gastroenterology 2003; 124:917-924; Hanauer et al. Gastroenterology 1999; 116:A731; and Hanauer et al. Clin Gastroenterol Hepatol 2004; 2:542-553). In certain instances, some patients who are administered a TNFα inhibitor for the treatment of Crohn's disease and respond to said treatment, may eventually lose their response to the first TNFα inhibitor. In other patient populations, intolerance to a certain TNFα inhibitor may be marked from the initial administration of the TNFα inhibitor. In one embodiment, the invention provides use of a TNFα inhibitor in the manufacture of a medicament for inducing remission of Crohn's disease in a subject who has lost response to or is intolerant to a different TNFα inhibitor. In one embodiment, the TNFα inhibitor which the subject has lost response to or is intolerant to is infliximab.

In one embodiment, the invention also provides methods and compositions for use in a subject who has not previously been administered infliximab. Thus, in one to embodiment, the methods and compositions of the invention are directed to a subpopulation of Crohn's patients who have not previously received infliximab.

In one embodiment, the invention provides an article of manufacture comprising adalimumab and a package insert, wherein the package insert indicates that adalimumab may be used to treat Crohn's disease in patients who have had an inadequate response to conventional therapy and/or who have lost response to or are intolerant to infliximab.

### Articles of Manufacture

The invention also provides a packaged pharmaceutical composition wherein the TNFα□ inhibitor, e.g., TNFα□ antibody, is packaged within a kit or an article of manufacture. The kit or article of manufacture of the invention contains materials useful for the treatment, including induction and/or remission, prevention and/or diagnosis of Crohn's disease. The kit or article of manufacture comprises a container and a label or package insert or printed material on or associated with the container which provides information regarding use of the TNFα inhibitor, e.g., a TNFα antibody, for the treatment of Crohn's disease.

A kit or an article of manufacture refers to a packaged product comprising components with which to administer a TNFα inhibitor for treatment of a Crohn's disease. The kit preferably comprises a box or container that holds the components of the kit. The box or container is affixed with a label or a Food and Drug Administration approved label, including a protocol for administering the TNFα inhibitor. The box or container holds components of the invention which are preferably contained within plastic, polyethylene, polypropylene, ethylene, or propylene vessels. The vessels can be capped-tubes or bottles. The kit can also include instructions for administering the TNFα antibody of the invention. In one embodiment the kit of the invention includes the formulation comprising the human antibody adalimumab (or D2E7), as described in PCT/IB03/04502 and U.S. application Ser. No. 10/222,140, incorporated by reference herein.

The term “package insert” is used to refer to instructions customarily included in commercial packages of therapeutic products, that contain information about the indications, usage, dosage, administration, contraindications and/or warnings concerning the use of such therapeutic products.

In one embodiment, the article of manufacture of the invention comprises (a) a first container with a composition contained therein, wherein the composition comprises a TNFα antibody; and (b) a package insert indicating that the TNFα antibody may be used for reducing signs and symptoms and inducing and maintaining remission of Crohn's disease. In a preferred embodiment, the label or package insert indicates that the TNFα inhibitor, e.g., a TNFα antibody, is used for inducing and maintaining remission Crohn's disease.

Suitable containers for the TNFα inhibitor, e.g., a TNFα antibody, include, for example, bottles, vials, syringes, pens, etc. The containers may be formed from a variety of materials such as glass or plastic. The container holds a composition which is by itself or when combined with another composition effective for treating, preventing and/or diagnosing the condition and may have a sterile access port.

In one embodiment, the article of manufacture comprises a TNFα inhibitor, e.g., a TNFα antibody, and a label which indicates to a subject who will be administering the TNFα inhibitor about using the TNFα inhibitor for the treatment of Crohn's disease. The label may be anywhere within or on the article of manufacture. In one embodiment, the article of manufacture comprises a container, such as a box, which comprises the TNFα inhibitor and a package insert or label providing information pertaining to use of the TNFα inhibitor for the treatment of Crohn's disease. In another embodiment, the information is printed on a label which is on the outside of the article of manufacture, in a position which is visible to prospective purchasers.

In one embodiment, the package insert of the invention informs a reader, including a subject who will be administering the TNFα inhibitor for treatment, that the TNFα inhibitor, e.g., a TNFα antibody such as adalimumab, is an indicated treatment of Crohn's disease, including of moderately to severely active disease in adult patients.

In one embodiment, the package insert describes certain patient populations who may respond favorably to the TNFα inhibitor within the article of manufacture. For example, the package insert may indicate that the TNFα antibody, e.g., adalimumab, may be used to treat Crohn's disease in patients who have had an inadequate response to conventional therapy and/or who have lost response to or are intolerant to infliximab. In another embodiment, the label of the invention indicates that adalimumab is indicated for treatment of moderately to severely active Crohn's disease in adult patients who have had an inadequate response to conventional therapy. In another embodiment, the label of the invention indicates that the TNFα inhibitor, e.g., a TNFα antibody such as adalimumab, is also indicated for treatment in adult patients with moderately to severely active Crohn's disease who have lost response to or are intolerant to infliximab.

In one embodiment, the package insert of the invention describes certain therapeutic benefits of the TNFα antibody, e.g., adalimumab, including specific symptoms of Crohn's disease which may be reduced by using the TNFα antibody, e.g., adalimumab. It should be noted that the package insert may also contain information pertaining to other disorders which are treatable using the TNFα antibody, e.g., adalimumab. Information described herein which is provided in a package insert and pertains to other disorders, i.e., diseases other than Crohn's disease, is also included within the scope of the invention. The package insert of the invention may indicate that extra TNFα in your body can attack normal healthy body tissues and cause inflammation especially in the tissues in your bones, cartilage, joints and digestive tract. The package insert of the invention may also indicate that adalimumab helps reduce the signs and symptoms of immune diseases, including rheumatoid and psoriatic arthritis (pain and swollen joints), ankylosing spondylitis (morning stiffness and back pain), and Crohn's disease (abdominal pain and diarrhea).

In another embodiment, the package insert of the invention describes the dose and administration of adalimumab, for the treatment of Crohn's disease. The label may indicate that the initiation of therapy includes a 160 mg dose at week 0 and 80 mg at week 2. The label may also indicate that the maintenance dosing for the treatment of Crohn's disease with adalimumab is 40 mg every other week. In one embodiment, the package insert indicates that the week 0 dose may be administered as 4 injections in one day or divided over 2 days. The label may also indicate that some patients with Crohn's disease may derive additional benefit by increasing frequency to 40 mg every week. In another embodiment, the package insert of the invention indicates that adalimumab is administered by subcutaneous injection.

In another embodiment, the label of the invention indicates that the recommended TNFα inhibitor, e.g., a TNFα antibody such as adalimumab, dose regimen for adult patients with Crohn's disease is 160 mg at week 0 (dose can be administered as four injections in one day or as two injections per day for two consecutive days), 80 mg at week 2, followed by 40 mg every other week beginning at week 4. The label of the invention may also indicate that some patients may derive additional benefit from increasing the dosing frequency of the TNFα inhibitor, e.g., a TNFα antibody such as adalimumab from 40 mg every other week to 40 mg every week.

The package insert of the invention may also provide information to subjects who will be receiving adalimumab regarding combination uses for both safety and efficacy purposes. In another embodiment, the label of the invention indicates that aminosalicylates, corticosteroids, and/or immunomodulatory agents (e.g., 6-mercaptopurine and azathioprine) may be continued during treatment with the TNFα inhibitor, e.g., a TNFα antibody, including adalimumab. In one embodiment, the invention provides an article of manufacture comprising a packaging material; a TNFα antibody, or antigen-binding portion thereof; and a label or package insert contained within the packaging material indicating that aminosalicylates, corticosteroids, and/or immunomodulatory agent, e.g., 6-mercaptopurine and azathioprine, may be continued during treatment with the TNFα antibody, or antigen-binding portion thereof.

The package insert of the invention may contain warnings and precautions regarding the use of the TNFα inhibitor, e.g., a TNFα antibody such as adalimumab. In one embodiment, the information provided in the label describes malignancies. The label of the invention may indicate that during the controlled portions of to TNFα antibody, such as adalimumab, trials in patients with rheumatoid arthritis, psoriatic arthritis, ankylosing spondylitis, and Crohn's disease, malignancies, other than lymphoma and non-melanoma skin cancer, were observed at a rate (95% confidence interval) of 0.6 (0.3, 1.0)/100 patient-years among 2887 adalimumab-treated patients versus a rate of 0.4 (0.2, 1.1)/100 patient-years among 1570 control patients (median duration of treatment of 5.7 months for adalimumab-treated patients and 5.5 months for control-treated patients). The label may also indicate that the size of the control group and limited duration of the controlled portions of studies precludes the ability to draw firm conclusions. In one embodiment, the label indicates that in the controlled and uncontrolled open-label portions of the clinical trials of adalimumab, the more frequently observed malignancies, other than lymphoma and non-melanoma skin cancer, were breast, colon, prostate, lung and melanoma. In one embodiment, the label indicates that these malignancies in adalimumab treated and control-treated patients were similar in type and number to what would be expected in the general population. The label may further indicate that during the controlled portions of adalimumab rheumatoid arthritis, psoriatic arthritis, ankylosing spondylitis, and Crohn's disease trials, the rate (95% confidence interval) of non-melanoma skin cancers was 0.8 (0.47, 1.24)/100 patient-years among adalimumab-treated patients 0.2 (0.05, 0.82)/100 patient-years among control patients. In one embodiment, the label indicates that the potential role of TNF blocking therapy in the development of malignancies is not known. In one embodiment, the label indicates that in the controlled portions of clinical trials of all the TNF-blocking agents, more cases of lymphoma have been observed among patients receiving TNF blockers compared to control patients. In one embodiment, the label indicates that in controlled trials in patients with rheumatoid arthritis, psoriatic arthritis, ankylosing spondylitis, and Crohn's disease, 2 lymphomas were observed among 2887 HUMIRA®-treated patients versus 1 among 1570 control patients. In one embodiment, the label of the invention indicates that in combining the controlled and uncontrolled open-label portions of these clinical trials with a median duration of approximately 2 years, including 4843 patients and over 13,000 patient-years of therapy, the observed rate of lymphomas is approximately 0.12/100 patient-years, and that this is approximately 3.5-fold higher than expected in the general population.

The label of the invention may contain information regarding the use of the TNFα inhibitor, e.g., a TNFα antibody such as adalimumab, in clinical studies for Crohn's disease. In one embodiment, the label of the invention describes the studies described herein as Example 1, either as a whole or in portion. The label of the invention may also indicate that adalimumab has been studied in over 1400 patients with Crohn's disease in four placebo-controlled and two open-label extension studies. The label of the invention may also indicate that the safety profile for patients with Crohn's to disease treated with HUMIRA® was similar to the safety profile seen in patients with rheumatoid arthritis.

The label of the invention may contain information regarding the pharmacodynamics of the TNFα inhibitor, e.g., a TNFα antibody such as adalimumab. In one embodiment, the label of the invention indicates that after treatment with adalimumab, a rapid decrease in levels of acute phase reactants of inflammation (C-reactive protein (CRP) and erythrocyte sedimentation rate (ESR) and serum cytokines (IL-6) was observed compared to baseline in patients with rheumatoid arthritis. In one embodiment, the label of the invention indicates that a rapid decrease in CRP levels was also observed in patients with Crohn's disease. The label may further indicate that serum levels of matrix metalloproteinases (MMP-1 and MMP-3) that produce tissue remodeling responsible for cartilage destruction were also decreased after adalimumab administration.

The label of the invention may also contain information regarding the pharmacokinetics of the TNFα inhibitor, e.g., a TNFα antibody such as adalimumab. In one embodiment, the label of the invention indicates that in patients with Crohn's disease, the loading dose of 160 mg adalimumab on week 0 followed by 80 mg adalimumab on week 2 achieves serum adalimumab trough concentrations of approximately 12 μg/mL. The label of the invention may also indicate that mean steady-state trough levels of approximately 7 μg/mL were observed in Crohn's disease patients who received a maintenance dose of 40 mg adalimumab every other week

In one embodiment, the invention provides an article of manufacture comprising a TNFα inhibitor and a package insert, wherein the package insert indicates that in patients with Crohn's disease who have been administered the TNFα inhibitor, the loading dose on week 0 followed by a second dose on week 2 achieves serum adalimumab trough concentrations of approximately 12 μg/mL.

In one embodiment, an article of manufacture comprising a TNFα inhibitor and a package insert, wherein the package insert indicates that in patients with Crohn's disease who have been administered the TNFα inhibitor, the mean steady-state trough levels of approximately 7 μg/mL were observed in Crohn's disease patients who received a maintenance dose of the TNFα inhibitor every other week.

The label of the invention may also contain information regarding drug interactions of the TNFα inhibitor, e.g., a TNFα antibody such as adalimumab, with other drugs. In one embodiment, the label indicates that methotrexate (MTX) reduced adalimumab apparent clearance after single and multiple dosing by 29% and 44% respectively, in patients with rheumatoid arthritis.

In one embodiment of the invention, the kit comprises a TNFα inhibitor, such as an antibody, an second pharmaceutical composition comprising an additional therapeutic to agent, and instructions for administration of both agents for the treatment of Crohn's disease. The instructions may describe how, e.g., subcutaneously, and when, e.g., at week 0, week 2, and biweekly thereafter, doses of TNFα antibody and/or the additional therapeutic agent shall be administered to a subject for treatment.

Another aspect of the invention pertains to kits containing a pharmaceutical composition comprising an anti-TNFα antibody and a pharmaceutically acceptable carrier and one or more additional pharmaceutical compositions each comprising a drug useful for treating a TNFα related disorder and a pharmaceutically acceptable carrier. Alternatively, the kit comprises a single pharmaceutical composition comprising an anti-TNFα antibody, one or more drugs useful for treating a TNFα related disorder and a pharmaceutically acceptable carrier. The kits further contain instructions for dosing of the pharmaceutical compositions for the treatment of a TNFα related disorder.

The package or kit alternatively may contain the TNFα inhibitor and it may be promoted for use, either within the package or through accompanying information, for the uses or treatment of the disorders described herein. The packaged pharmaceuticals or kits further can include a second agent (as described herein) packaged with or copromoted with instructions for using the second agent with a first agent (as described herein).

### V. Additional Therapeutic Agents

TNFα inhibitors, including TNFα antibodies, or antigen binding portions thereof, may be used in the methods, uses, and compositions of the invention either alone or in combination with an additional therapeutic agent. It should be understood that the TNFα inhibitors can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the TNFα inhibitors. The additional agent also can be an agent that imparts a beneficial attribute to the therapeutic composition, e.g., an agent which effects the viscosity of the composition.

It should further be understood that the combinations which are to be included within this invention are those combinations useful for their intended purpose. The agents set forth below are illustrative for purposes and not intended to be limited. The combinations, which are part of this invention, can be the TNFα inhibitors of the present invention and at least one additional agent selected from the lists below. The combination can also include more than one additional agent, e.g., two or three additional agents if the combination is such that the formed composition can perform its intended function.

Non-limiting examples of therapeutic agents for Psoriasis with which an antibody, or antibody portion, of the invention can be combined include the following: small molecule inhibitor of KDR (ABT-123), small molecule inhibitor of Tie-2, calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, sulfasalazine.

TNFα inhibitors described herein may be used in combination with additional therapeutic agents such as a Disease Modifying Anti-Rheumatic Drug (DMARD) or a Nonsteroidal Antiinflammatory Drug (NSAID) or a steroid or any combination thereof.

Preferred examples of a DMARD are hydroxychloroquine, leflunomide, methotrexate, parenteral gold, oral gold and sulfasalazine. Preferred examples of non-steroidal anti-inflammatory drug(s) also referred to as NSAIDS include drugs like ibuprofen. Other preferred combinations are corticosteroids including prednisolone; the well known side effects of steroid use can be reduced or even eliminated by tapering the steroid dose required when treating patients in combination with TNFα inhibitors of this invention.

Preferred combinations of therapeutic agents may interfere at different points in the autoimmune and subsequent inflammatory cascade; preferred examples include TNF antagonists such as soluble p55 or p75 TNF receptors, derivatives, thereof, (p75TNFR1gG (Enbrel™) or p55TNFR1gG (Lenercept), chimeric, humanized or human TNF antibodies, or a fragment thereof, including infliximab (Remicade®, Johnson and Johnson; described in U.S. Pat. No. 5,656,272, incorporated by reference herein), PSORIASIS P571 (a humanized monoclonal anti-TNF-alpha IgG4 antibody), PSORIASIS P 870 (a humanized monoclonal anti-TNF-alpha antibody fragment), an anti-TNF dAb (Peptech), CNTO 148 (golimumab; Medarex and Centocor, see WO 02/12502), and adalimumab (HUMIRA®® Abbott Laboratories, a human anti-TNF mAb, described in U.S. Pat. No. 6,090,382 as D2E7). Additional TNF antibodies which can be to used in the invention are described in U.S. Pat. Nos. 6,593,458; 6,498,237; 6,451,983; and 6,448,380, each of which is incorporated by reference herein. Other combinations including TNFα converting enzyme (TACE) inhibitors; IL-1 inhibitors (Interleukin-1-converting enzyme inhibitors, IL-1RA etc.) may be effective for the same reason. Other preferred combinations include Interleukin 11. Yet another preferred combination are other key players of the autoimmune response which may act parallel to, dependent on or in concert with TNFα inhibitors function; especially preferred are IL-18 antagonists including IL-18 antibodies or soluble IL-18 receptors, or IL-18 binding proteins. Yet another preferred combination are non-depleting anti-PSORIASIS 4 inhibitors. Yet other preferred combinations include antagonists of the co-stimulatory pathway PSORIASIS 80 (B7.1) or PSORIASIS 86 (B7.2) including antibodies, soluble receptors or antagonistic ligands.

The TNFα inhibitors used in the invention may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα□ or IL-1 (e.g. IRAK, NIK, IKK, p38 or MAP kinase inhibitors), IL-1β converting enzyme inhibitors, TNFα □converting enzyme (TACE) inhibitors, T-cell signalling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™ and p55TNFRIgG (Lenercept)), sIL-1RI, sIL-1RII, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone hcl, hydrocodone bitartrate/apap, diclofenac sodium/misoprostol, fentanyl, anakinra, human recombinant, tramadol hcl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline hcl, sulfadiazine, oxycodone hcl/acetaminophen, olopatadine hcl, misoprostol, naproxen sodium, omeprazole, cyclophosphamide, rituximab, IL-1 TRAP, MRA, CTLA4-IG, IL-18 BP, anti-IL-18, Anti-IL15, BIRB-796, SCIO-469, VX-702, AMG-548, VX-740, Roflumilast, IC-485, PSORIASIS C-801, and Mesopram.

Non-limiting examples of therapeutic agents for psoriasis with which TNFα inhibitor of the invention can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL-1, IL-2, IL-6 (including Actemra (tocilizumab), IL-7, IL-8, IL-15, IL-16, IL-17, IL-18, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

The antibodies of the invention, or antigen binding portions thereof, may also be combined with agents, such as methotrexate, cyclosporin, FK506, rapamycin, mycophenolate mofetil, leflunomide, NSAIDs, for example, ibuprofen, corticosteroids such as prednisolone, phosphodiesterase inhibitors, adenosine agonists, antithrombotic agents, complement inhibitors, adrenergic agents, agents which interfere with signalling by proinflammatory cytokines such as TNFα or IL-1 (e.g. IRAK, NIK, IKK, p38 or MAP kinase inhibitors), IL-1β converting enzyme inhibitors, TNFα converting enzyme inhibitors, T-cell signalling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-11, IL-13 and TGFβ).

Additional examples of therapeutic agents for psoriasis in which a TNFα inhibitor can be combined include the following: combinations of TNF antagonists, for example, anti-TNF antibodies, D2E7 (PCT Publication No. WO 97/29131; HUMIRA®), CA2 (REMICADE), PSORIASIS P 571, TNFR-Ig constructs, (p75TNFRIgG (ENBREL) and p55TNFRIgG (LENERCEPT)) inhibitors and PDE4 inhibitors. TNFα inhibitors of the invention can be combined with corticosteroids, for example, budenoside and dexamethasone. TNFα inhibitors of the invention may also be combined with agents such as sulfasalazine, 5-aminosalicylic acid and olsalazine, and agents which interfere with synthesis or action of proinflammatory cytokines such as IL-1, for example, IL-1β converting enzyme inhibitors and IL-1ra. TNFα inhibitors may also be used with T cell signaling inhibitors, for example, tyrosine kinase inhibitors 6-mercaptopurines. TNFα inhibitors can be combined with IL-11. TNFα inhibitors can be combined with mesalamine, prednisone, azathioprine, mercaptopurine, infliximab, methylprednisolone sodium succinate, diphenoxylate/atrop sulfate, loperamide hydrochloride, methotrexate, omeprazole, folate, ciprofloxacin/dextrose-water, hydrocodone bitartrate/apap, tetracycline hydrochloride, fluocinonide, metronidazole, thimerosal/boric acid, cholestyramine/sucrose, ciprofloxacin hydrochloride, hyoscyamine sulfate, meperidine hydrochloride, midazolam hydrochloride, oxycodone hcl/acetaminophen, promethazine hydrochloride, sodium phosphate, sulfamethoxazole/trimethoprim, celecoxib, polycarbophil, propoxyphene napsylate, hydrocortisone, multivitamins, balsalazide disodium, codeine phosphate/apap, colesevelam hcl, cyanocobalamin, folic acid, levofloxacin, methylprednisolone, natalizumab and interferon-gamma

The TNFα inhibitors may also be combined with agents, such as alemtuzumab, dronabinol, Unimed, daclizumab, mitoxantrone, xaliproden hydrochloride, fampridine, glatiramer acetate, natalizumab, sinnabidol, a-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist) MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists, IL-4 agonists, and the humanized IL-6 antibody tocilizumab.

In yet another embodiment, the invention includes an article of manufacture or a method comprising the combination of a TNF inhibitor and an antibiotic or antiinfective agent. Antiinfective agents include those agents known in the art to treat viral, fungal, parasitic or bacterial infections. The term, “antibiotic,” as used herein, refers to a chemical substance that inhibits the growth of, or kills, microorganisms. Encompassed by this term are antibiotic produced by a microorganism, as well as synthetic antibiotics (e.g., analogs) known in the art. Antibiotics include, but are not limited to, clarithromycin (Biaxin®), ciprofloxacin (Cipro®), and metronidazole (Flagyl®).

Any one of the above-mentioned therapeutic agents, alone or in combination therewith, can be administered to a subject suffering from a TNFα-related disorder in which TNFα is detrimental, in combination with the TNFα antibody using a multiple variable dose treatment regimen. In one embodiment, any one of the above-mentioned therapeutic agents, alone or in combination therewith, can be administered to a subject suffering from an intestinal disorder in addition to a TNFα antibody to treat another to TNFα-related disorder, such as rheumatoid arthritis. It should be understood that the additional therapeutic agents can be used in combination therapy as described above, but also may be used in other indications described herein wherein a beneficial effect is desired.

The combination of agents used within the methods and pharmaceutical compositions described herein may have a therapeutic additive or synergistic effect on the condition(s) or disease(s) targeted for treatment. The combination of agents used within the methods or pharmaceutical compositions described herein also may reduce a detrimental effect associated with at least one of the agents when administered alone or without the other agent(s) of the particular pharmaceutical composition. For example, the toxicity of side effects of one agent may be attenuated by another agent of the composition, thus allowing a higher dosage, improving patient compliance, and improving therapeutic outcome. The additive or synergistic effects, benefits, and advantages of the compositions apply to classes of therapeutic agents, either structural or functional classes, or to individual compounds themselves.

### VI. Efficacy of TNFα Inhibitor

### Psoriasis

The invention also provides methods for determining whether a TNFα inhibitor is effective at treating psoriasis in a subject. Such methods may be used to determine the efficacy of a TNFα inhibitor, including those which are unknown or unconfirmed to have such efficacy. Using the methods described herein, effective TNFα inhibitors may be determined or confirmed, and, subsequently, used in the method of treating psoriasis. Further methods for determining whether a TNFα inhibitor is effective at treating psoriasis in a subject are described in U.S. Provisional Application Nos. 60/832,370 (filed Jul. 20, 2006), 60/851,830 (filed Oct. 6, 2006), and 60/857,352 (filed Nov. 6, 2006), each of which are incorporated herein by reference.

It should be noted that the Examples provided herein represent different methods of determining the efficacy of a TNFα inhibitor, such as a human TNFα antibody, or antigen-binding portion thereof. As such, data and results described in the Examples section which shows efficacy of a TNFα inhibitor, e.g., ability to maintain remission of psoriasis, are included in the methods of determining efficacy of the invention. Time points for determining efficacy will be understood by those of skill in the art to depend on the type of efficacy being determined, e.g., maintenance of remission.

In one embodiment, measurements in scores, e.g., the PASI response or PGA score of a subject, may be measured against a subject's baseline score. Generally, a baseline refers to a measurement or score of a patient before treatment, i.e. week 0. Other time points may also be included as a starting point in determining efficacy, however. For example, in determining the efficacy of a TNFα inhibitor for treating psoriasis in a patient population, a determination of the percentage of the patient population who were achieved a response, i.e., PASI 75 response, may be determined based on a time point from when remission was induced.

Patient populations described in the methods of the invention are generally selected based on common characteristics, such as, but not limited to, subjects diagnosed with psoriasis who are in remission as a result of being on a dosing regimen comprising a TNFα inhibitor. Such a patient population would be appropriate for determining the efficacy of the TNFα inhibitor for maintaining remission in psoriasis in the given patient population. In one embodiment, the patient population is an adult population, e.g., older than 17 years of age or older than 18 years of age.

In one embodiment, the methods of the invention for determining whether a TNFα inhibitor is an effective TNFα inhibitor, include determining changes, improvements, measurements, etc., in psoriasis using appropriate indices known in the art, e.g., PASI, from a patient population who has already been administered the TNFα inhibitor. Such a patient population may be pre-selected according to common characteristics, e.g., psoriasis, loss of response to infliximab, and may have already been given the TNFα inhibitor. Administration of the TNFα inhibitor may or may not be performed by the same person of ordinary skill who is determining the efficacy of the TNFα inhibitor in accordance with the teachings of the specification.

In one embodiment, the methods of the invention for determining whether a TNFα inhibitor is an effective TNFα inhibitor, include determining changes, improvements, measurements, etc., in psoriasis using appropriate indices known in the art, e.g., PASI, PGA, DLQI, status of psoriasis related disorders, etc. from a patient population who has already been administered the TNFα inhibitor. Such a patient population may be pre-selected according to common characteristics, e.g., psoriasis, loss of response to infliximab, and may have already been given the TNFα inhibitor. Administration of the TNFα inhibitor may or may not be performed by the same person of ordinary skill who is determining the efficacy of the TNFα inhibitor in accordance with the teachings of the specification.

Methods of the invention relating to determining efficacy, i.e., determining whether a TNFα inhibitor is an effective TNFα inhibitor, may also be applied to specific patient populations within the overall patient population who together have specific, common characteristics, i.e., a subpopulation. For example, the patient population may comprise patients on concomitant immunosuppressant (IMM) treatment with the TNFα inhibitor. In another example, the patient population may comprises patients not on concomitant IMM treatment.

In one embodiment, the invention provides a method for determining the efficacy of a TNFα inhibitor, including a human TNFα antibody, for treating psoriasis in a subject, using the Psoriasis Area Severity Index (PASI). The Psoriasis Area and Severity Index (PASI) is used by dermatologists to assess psoriasis disease intensity. This index is based on the quantitative assessment of three typical signs of psoriatic lesions: erythema, infiltration, and desquamation, combined with the skin surface area involvement. Since its development in 1978, this instrument has been used throughout the world by clinical investigators (Fredriksson T, Petersson U: Severe psoriasis—oral therapy with a new retinoid. Dermatologica 1978; 157: 238-41.) PASI is indicated as PASI 50 (a 50 percent improvement in PASI from baseline), PASI 75 (a 75 percent improvement in PASI from baseline), PASI 90 (a 90 percent improvement in PASI from baseline), and PASI 100 (a 100 percent improvement in PASI from baseline). The efficacy of a TNFα inhibitor for treatment of psoriatic arthritis in a patient population who has psoriasis, may be evaluated by determining the percentage of the patient population in whom a PASI 50, PASI 75, PASI 90, or PASI 100 response has been achieved following administration of the TNFα inhibitor.

The Physicians Global Assessment (PGA) is used to assess psoriasis activity and follow clinical response to treatment. It is a six-point score that summarizes the overall quality (erythema, scaling and thickness) and extent of plaques relative to the baseline assessment. A patient's response is rated as worse, poor (0-24%), fair (25-49%), good (50-74%), excellent (75-99%), or cleared (100%) (van der Kerkhof P. The psoriasis area and severity index and alternative approaches for the assessment of severity: persisting areas of confusion. Br J Dermatol 1997; 137:661-662).

The DLQI is an additional validated instrument used to assess dermatologic-related functional limitations. Characteristics of the DLQI include:
ten items on an overall scoring range of 0-30; higher scores represent greater quality of life impairment and lower scores represent lower quality of life impairment;
well-established properties of reliability and validity for the DLQI total score in a dermatology setting (see Badia et al. (1999) Br J Dermatol 141:698; Finlay et al. (1994) Clin Exp Dermatol 19:210; and Shikier et al. (2003) Health and Quality of Life Outcomes 1:53);
six subcategories: symptoms and feelings; daily activities; leisure; work/school; personal relationships; and treatment;
all data are observed values. Patients who discontinued before the time point were not included in this analysis.
   Ranges of DLQI scores can be evaluated for their correspondence to categories of to disease impact.

The PASI, PGA, and DLQI scores may be used as an index for measuring efficacy of a TNFα inhibitor in a patient population having psoriasis, where attaining a certain percentage of patients within a population who were administered the TNFα inhibitor and who maintain clinical remission, i.e. PASI<50 or PASI<75, indicates that the TNFα inhibitor is effective for treating of psoriasis. In one embodiment, the invention provides a method for determining whether a human TNFα antibody is effective for treating psoriasis.

The efficacy of a TNFα inhibitor for treating psoriasis in a patient population, i.e., PASI 75 response (also referred to herein as a PASI/PASI75 score), may be evaluated by determining the percentage of the patient population in treatment of psoriasis has been effective following administration of the TNFα inhibitor.

In one embodiment, the invention provides a method of determining the efficacy of a TNFα inhibitor for treating psoriasis in a subject comprising determining a Psoriasis Area Severity Index (PASI) score of a patient population having psoriasis and who was administered the TNFα inhibitor, wherein a PASI 75 response is achieved in at least about 77% of the patient population indicates that the TNFα inhibitor is an effective TNFα inhibitor for the treatment of psoriasis in a subject. In one embodiment, the method further comprises administering the effective TNFα inhibitor to a subject to treat psoriasis. The invention provides a method of treating psoriasis in a subject comprising administering an effective amount of a TNFα inhibitor to the subject such that treatment of psoriasis is maintained, wherein the effective human TNFα antibody was previously identified as achieving a PASI 75 response in at least about 77% of a patient population having psoriasis and a baseline PASI<10.

In one embodiment, the invention provides a method of treating psoriasis in a subject comprising administering an effective amount of a human TNFα antibody to the subject such that psoriasis is treated, wherein the effective human TNFα antibody was previously identified as achieving a PASI 75 response in at least about 77% of a patient population having psoriasis and a baseline PASI<10.

In one embodiment, a PASI 75 response is achieved in at least about 77% of the patient population indicates that the human TNFα antibody is an effective human TNFα antibody for the treatment of psoriasis in a subject. In one embodiment, a PASI 75 response is achieved in at least about 80% of the patient population indicates that the human TNFα antibody is an effective human TNFα antibody for the treatment of psoriasis in a subject. In one embodiment, a PASI 75 response is achieved in at least about 85% of the patient population indicates that the human TNFα antibody is an effective human TNFα antibody for the treatment of psoriasis in a subject. In one embodiment, a PASI 75 response is achieved in at least about 88% of the patient population indicates that the human TNFα antibody is an effective human TNFα antibody for the treatment of psoriasis in a subject. In one embodiment, a PASI 75 response is achieved in at least about 90% of the patient population indicates that the human TNFα antibody is an effective human TNFα antibody for the treatment of psoriasis in a subject.

Numbers intermediate to the above recited percentages, e.g., 78%, 79%, 80%. 81%, 82%, 83%, 84%, 85%, 86%, 87%, 88%, and 89%, as well as all other numbers recited herein, are also intended to be part of this invention. Ranges of values using a combination of any of the above recited values as upper and/or lower limits are intended to be included in the scope of the invention. For example, in one embodiment a PASI 75 response score of in at least between 77% and 90% of the patient population indicates that the TNFα inhibitor is an effective TNFα inhibitor for the treatment of psoriasis in a subject.

In one embodiment, the invention provides a method of determining the efficacy of a TNFα inhibitor for treating psoriasis in a subject comprising determining a Psoriasis Area Severity Index (PASI) score of a patient population having psoriasis and who was administered the TNFα inhibitor, wherein a PASI 75 response is achieved in at least about 32% of the patient population indicates that the TNFα inhibitor is an effective TNFα inhibitor for the treatment of psoriasis in a subject. In one embodiment, the method further comprises administering the effective TNFα inhibitor to a subject to treat psoriasis. The invention provides a method of treating psoriasis in a subject comprising administering an effective amount of a TNFα inhibitor to the subject such that treatment of psoriasis is maintained, wherein the effective human TNFα antibody was previously identified as achieving a PASI 75 response in at least about 32% of a patient population having psoriasis and a baseline PASI<10.

In one embodiment, a PASI 75 response is achieved in at least about 32% of the patient population indicates that the human TNFα antibody is an effective human TNFα antibody for the treatment of psoriasis in a subject. In one embodiment, a PASI 75 response is achieved in at least about 40% of the patient population indicates that the human TNFα antibody is an effective human TNFα antibody for the treatment of psoriasis in a subject. In one embodiment, a PASI 75 response is achieved in at least about 50% of the patient population indicates that the human TNFα antibody is an effective human TNFα antibody for the treatment of psoriasis in a subject. In one embodiment, a PASI 75 response is achieved in at least about 60% of the patient population indicates that the human TNFα antibody is an effective human TNFα antibody for the treatment of psoriasis in a subject. In one embodiment, a PASI 75 response is achieved in at least about 70% of the patient population indicates that the human TNFα antibody is an effective human TNFα antibody for the treatment of to psoriasis in a subject. In one embodiment, a PASI 75 response is achieved in at least about 80% of the patient population indicates that the human TNFα antibody is an effective human TNFα antibody for the treatment of psoriasis in a subject. In one embodiment, a PASI 75 response is achieved in at least about 90% of the patient population indicates that the human TNFα antibody is an effective human TNFα antibody for the treatment of psoriasis in a subject.

Numbers intermediate to the above recited percentages, e.g., 32%, 33%, 34%. 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, and 89%, as well as all other numbers recited herein, are also intended to be part of this invention. Ranges of values using a combination of any of the above recited values as upper and/or lower limits are intended to be included in the scope of the invention. For example, in one embodiment a PASI 75 response score of in at least between 32% and 90% of the patient population indicates that the TNFα inhibitor is an effective TNFα inhibitor for the treatment of psoriasis in a subject.

In one embodiment, a PASI 90 response is achieved in at least about 63% of the patient population indicates that the human TNFα antibody is an effective human TNFα antibody for achieving a clinical response in psoriasis in a subject. In one embodiment, a PASI 90 response is achieved in at least about 65% of the patient population indicates that the human TNFα antibody is an effective human TNFα antibody for achieving a clinical response in psoriasis in a subject. In one embodiment, a PASI 90 response is achieved in at least about 68% of the patient population indicates that the human TNFα antibody is an effective human TNFα antibody for achieving a clinical response in psoriasis in a subject. In one embodiment, a PASI 90 response is achieved in at least about 70% of the patient population indicates that the human TNFα antibody is an effective human TNFα antibody for achieving a clinical response in psoriasis in a subject. In one embodiment, a PASI 90 response is achieved in at least about 75% of the patient population indicates that the human TNFα antibody is an effective human TNFα antibody for achieving a clinical response in psoriasis in a subject. In one embodiment, a PASI 90 response is achieved in at least about 80% of the patient population indicates that the human TNFα antibody is an effective human TNFα antibody for achieving a clinical response in psoriasis in a subject.

Numbers intermediate to the above recited percentages, e.g., 63%, 64%, 65%. 66%, 67%, 68%, 69%, 70%, 71%, 72%, 73%, 74%, 75%, 76%, 77%, 78%, 79%, and 80%, as well as all other numbers recited herein, are also intended to be part of this to invention. Ranges of values using a combination of any of the above recited values as upper and/or lower limits are intended to be included in the scope of the invention. For example, in one embodiment a PASI 90 response score of in at least between 63% and 80% of the patient population indicates that the TNFα inhibitor is an effective TNFα inhibitor for the treatment of psoriasis in a subject.

In one embodiment, a PASI 90 response is achieved in at least about 25% of the patient population indicates that the human TNFα antibody is an effective human TNFα antibody for achieving a clinical response in psoriasis in a subject. In one embodiment, a PASI 90 response is achieved in at least about 30% of the patient population indicates that the human TNFα antibody is an effective human TNFα antibody for achieving a clinical response in psoriasis in a subject. In one embodiment, a PASI 90 response is achieved in at least about 40% of the patient population indicates that the human TNFα antibody is an effective human TNFα antibody for achieving a clinical response in psoriasis in a subject. In one embodiment, a PASI 90 response is achieved in at least about 50% of the patient population indicates that the human TNFα antibody is an effective human TNFα antibody for achieving a clinical response in psoriasis in a subject. In one embodiment, a PASI 90 response is achieved in at least about 60% of the patient population indicates that the human TNFα antibody is an effective human TNFα antibody for achieving a clinical response in psoriasis in a subject. In one embodiment, a PASI 90 response is achieved in at least about 62% of the patient population indicates that the human TNFα antibody is an effective human TNFα antibody for achieving a clinical response in psoriasis in a subject.

Numbers intermediate to the above recited percentages, e.g., 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, and 61%, as well as all other numbers recited herein, are also intended to be part of this invention. Ranges of values using a combination of any of the above recited values as upper and/or lower limits are intended to be included in the scope of the invention. For example, in one embodiment a PASI 90 response score of in at least between 24% and 62% of the patient population indicates that the TNFα inhibitor is an effective TNFα inhibitor for the treatment of psoriasis in a subject.

In one embodiment, a PASI 100 response is achieved in at least about 38% of the patient population indicates that the human TNFα antibody is an effective human TNFα antibody for achieving a clinical response in psoriasis in a subject. In one embodiment, a PASI 100 response is achieved in at least about 40% of the patient population indicates that the human TNFα antibody is an effective human TNFα antibody for achieving a clinical response in psoriasis in a subject. In one to embodiment, a PASI 100 response is achieved in at least about 45% of the patient population indicates that the human TNFα antibody is an effective human TNFα antibody for achieving a clinical response in psoriasis in a subject. In one embodiment, a PASI 100 response is achieved in at least about 48% of the patient population indicates that the human TNFα antibody is an effective human TNFα antibody for achieving a clinical response in psoriasis in a subject. In one embodiment, a PASI 100 response is achieved in at least about 50% of the patient population indicates that the human TNFα antibody is an effective human TNFα antibody for achieving a clinical response in psoriasis in a subject.

Numbers intermediate to the above recited percentages, e.g., 36%, 37%, 38%. 39%, 40%, 41%, 42%, 43%, 44%, 45%, 46%, 47%, 48%, 49%, and 50%, as well as all other numbers recited herein, are also intended to be part of this invention. Ranges of values using a combination of any of the above recited values as upper and/or lower limits are intended to be included in the scope of the invention. For example, in one embodiment a PASI 100 response score of in at least between 36% and 50% of the patient population indicates that the TNFα inhibitor is an effective TNFα inhibitor for the treatment of psoriasis in a subject.

In one embodiment, a PASI 100 response is achieved in at least about 15% of the patient population indicates that the human TNFα antibody is an effective human TNFα antibody for achieving a clinical response in psoriasis in a subject. In one embodiment, a PASI 100 response is achieved in at least about 20% of the patient population indicates that the human TNFα antibody is an effective human TNFα antibody for achieving a clinical response in psoriasis in a subject. In one embodiment, a PASI 100 response is achieved in at least about 25% of the patient population indicates that the human TNFα antibody is an effective human TNFα antibody for achieving a clinical response in psoriasis in a subject. In one embodiment, a PASI 100 response is achieved in at least about 30% of the patient population indicates that the human TNFα antibody is an effective human TNFα antibody for achieving a clinical response in psoriasis in a subject. In one embodiment, a PASI 100 response is achieved in at least about 35% of the patient population indicates that the human TNFα antibody is an effective human TNFα antibody for achieving a clinical response in psoriasis in a subject.

Numbers intermediate to the above recited percentages, e.g., 15%, 16%, 17%, 18%, 19%, 20%, 21%, 22%, 23%, 24%, 25%, 26%, 27%, 28%, 29%, 30%, 31%, 32%, 33%, and 34% as well as all other numbers recited herein, are also intended to be part of this invention. Ranges of values using a combination of any of the above recited values as upper and/or lower limits are intended to be included in the scope of the invention. For example, in one embodiment a PASI 100 response score of in at least between 15% and 35% of the patient population indicates that the TNFα inhibitor is an effective TNFα inhibitor for the treatment of psoriasis in a subject.

In one embodiment the invention provides a method of determining the efficacy of a TNFα inhibitor for achieving a clinical response in psoriasis in a subject comprising determining a Physician's Global Assessment (PGA) score of a patient population having psoriasis who was administered the human TNFα antibody, wherein a PGA score of “clear” or “almost clear” in at least about 77% of the patient population indicates that the human TNFα antibody is an effective human TNFα antibody for treating psoriasis in a subject.

In one embodiment, the invention provides a method of treating psoriasis in a subject comprising administering an effective amount of a human TNFα antibody to the subject, wherein the effective human TNFα antibody was previously identified as maintaining a PGA score of “clear” or “almost clear” in at least about 77% of a patient population having psoriasis.

In one embodiment, a PGA score of “clear” or “almost clear” in at least about 77% of a patient population having psoriasis indicates that the human TNFα antibody is an effective human TNFα antibody for treating psoriasis in a subject. In one embodiment, a PGA score of “clear” or “almost clear” in at least about 80% of a patient population having psoriasis indicates that the human TNFα antibody is an effective human TNFα antibody for treating psoriasis in a subject.

Numbers intermediate to the above recited percentages, e.g., 78%, and 79%, as well as all other numbers recited herein, are also intended to be part of this invention. Ranges of values using a combination of any of the above recited values as upper and/or lower limits are intended to be included in the scope of the invention. For example, in one embodiment a PGA score of “clear” or “almost clear” in at least between 77% and 90% of the patient population indicates that the TNFα inhibitor is an effective TNFα inhibitor for the treatment of psoriasis in a subject.

In one embodiment the invention provides a method of determining the efficacy of a TNFα inhibitor for achieving a clinical response in psoriasis in a subject comprising determining a Physician's Global Assessment (PGA) score of a patient population having psoriasis who was administered the human TNFα antibody, wherein a PGA score of “clear” or “almost clear” in at least about 45% of the patient population indicates that the human TNFα antibody is an effective human TNFα antibody for treating psoriasis in a subject.

In one embodiment, the invention provides a method of treating psoriasis in a subject comprising administering an effective amount of a human TNFα antibody to the subject, wherein the effective human TNFα antibody was previously identified as maintaining a PGA score of “clear” or “almost clear” in at least about 76% of a patient population having psoriasis.

In one embodiment, a PGA score of “clear” or “almost clear” in at least about 45% of a patient population having psoriasis indicates that the human TNFα antibody is an effective human TNFα antibody for treating psoriasis in a subject. In one embodiment, a PGA score of “clear” or “almost clear” in at least about 76% of a patient population having psoriasis indicates that the human TNFα antibody is an effective human TNFα antibody for treating psoriasis in a subject.

Numbers intermediate to the above recited percentages, e.g., 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, and 75%, as well as all other numbers recited herein, are also intended to be part of this invention. Ranges of values using a combination of any of the above recited values as upper and/or lower limits are intended to be included in the scope of the invention. For example, in one embodiment a PGA score of “clear” or “almost clear” in at least between 45% and 76% of the patient population indicates that the TNFα inhibitor is an effective TNFα inhibitor for the treatment of psoriasis in a subject.

It should be noted that the Examples provided herein represent different methods of determining the efficacy of a TNFα inhibitor, such as a human TNFα antibody, or antigen-binding portion thereof. As such, data and results described in the Examples section which shows efficacy of a TNFα inhibitor, e.g., ability to maintain remission of psoriasis, are included in the methods of determining efficacy of the invention.

Time points for determining efficacy will be understood by those of skill in the art to depend on the type of efficacy being determined, e.g., maintenance of remission. In one embodiment, measurements in scores, e.g., the PASI response or PGA score of a subject, may be measured against a subject's baseline score. Generally, a baseline refers to a measurement or score of a patient before treatment, i.e. week 0. Other time points may also be included as a starting point in determining efficacy, however. For example, in determining the efficacy of a TNFα inhibitor for treating psoriasis in a patient population, a determination of the percentage of the patient population who were achieved a response, i.e., PASI 75 response, may be determined based on a time point from when remission was induced.

Patient populations described in the methods of the invention are generally selected based on common characteristics, such as, but not limited to, subjects diagnosed with psoriasis who are in remission as a result of being on a dosing regimen comprising a TNFα inhibitor. Such a patient population would be appropriate for determining the efficacy of the TNFα inhibitor for maintaining remission in psoriasis in the given patient population. In one embodiment, the patient population is an adult population, e.g., older than 17 years of age or older than 18 years of age.

In addition, while the above methods are described in terms of patient populations, methods of efficacy described herein may also be applied to individual subjects. For example, a method for determining efficacy may comprise determining whether a subject who has psoriasis, and who is on a dosage regimen comprising a human TNFα antibody, is able to achieve a PASI 75 response to determining if the human TNFα antibody is an effective human TNFα antibody. In one embodiment, if the subject is able to achieve a PASI 75 response for at least about 24 weeks, then the human TNFα antibody is effective at treating psoriasis.

The Examples and discoveries described herein are representative of a TNFα inhibitor, i.e., adalimumab, which is effective for treating psoriasis. As such, the studies and results described in the Examples section herein may be used as a guideline for determining the efficacy of a TNFα inhibitor, i.e., whether a TNFα inhibitor is an effective TNFα inhibitor for the treatment of psoriasis. In one embodiment, methods of determining efficacy described herein may be used to determine whether a TNFα inhibitor is bioequivalent to another TNFα inhibitor.

In one embodiment, the article of manufacture of the invention comprises instructions regarding how to determine the efficacy of the TNF inhibitor for the treatment of psoriasis.

Additional methods for assessing efficacy of a TNFα inhibitor for the treatment of psoriasis are described below.

### Crohn's Disease

The invention also provides methods for determining whether a TNFα inhibitor is effective at treating Crohn's disease in a subject. Such methods may be used to determine the efficacy of a TNFα inhibitor, including those which are unknown or unconfirmed to have such efficacy. Using the methods described herein, effective TNFα inhibitors may be determined or confirmed, and, subsequently, used in the method of treating Crohn's disease. Further methods for determining whether a TNFα inhibitor is effective at treating Crohn's disease in a subject are described in U.S. patent application Ser. No. 11/804,725 and in PCT/US2007/008962, each of which are hereby incorporated by reference.

It should be noted that the Examples provided herein represent different methods of determining the efficacy of a TNFα inhibitor, such as a human TNFα antibody, or antigen-binding portion thereof. As such, data and results described in the Examples section which shows efficacy of a TNFα inhibitor, e.g., ability to maintain remission of Crohn's, are included in the methods of determining efficacy of the invention.

Time points for determining efficacy will be understood by those of skill in the art to depend on the type of efficacy being determined, e.g., maintenance of remission. In one embodiment, measurements in scores, e.g., a decrease in the CDAI score of a subject, may be measured against a subject's baseline score. Generally, a baseline refers to a measurement or score of a patient before treatment, i.e. week 0. Other time points may also be included as a starting point in determining efficacy, however. For example, in determining the efficacy of a TNFα inhibitor for maintaining remission of Crohn's disease in a patient population, a determination of the percentage of the patient population who maintained remission, i.e., CDAI score of less than 150, may be determined based on a time point from when remission was induced.

Patient populations described in the methods of the invention are generally selected based on common characteristics, such as, but not limited to, subjects diagnosed with Crohn's disease who are in remission as a result of being on a dosing regimen comprising a TNFα inhibitor. Such a patient population would be appropriate for determining the efficacy of the TNFα inhibitor for maintaining remission in Crohn's disease in the given patient population. In one embodiment, the patient population is an adult population, .e.g, older than 17 years of age or older than 18 years of age.

In one embodiment, the invention provides a method for determining the efficacy of a TNFα inhibitor, including a human TNFα antibody, for maintaining remission of Crohn's disease in a subject, using the Crohn's Disease Activity Index (CDAI). The CDAI was developed to provide a single index of degree of illness in Crohn's disease, where index values of 150 and below are associated with quiescent disease; values above 150 indicate active disease, and values above 450 are seen with extremely severe disease (see Best et al. Gastroenterology. 1976 March; 70(3):439-44). The CDAI may be used as an index for measuring efficacy of a TNFα inhibitor in a patient population having Crohn's disease, where attaining a certain percentage of patients within a population who were administered the TNFα inhibitor and who maintain clinical remission, i.e. CDAI <150, indicates that the TNFα inhibitor is effective for maintaining remission of Crohn's disease. In one embodiment, the invention provides a method for determining whether a human TNFα antibody is effective for maintaining remission of Crohn's disease.

The efficacy of a TNFα inhibitor for maintaining remission of Crohn's disease in a patient population who has achieved remission, i.e., CDAI<150 (also referred to herein as a CDAI/CDAI score of less than 150), may be evaluated by determining the percentage of the patient population in whom remission of Crohn's disease has been induced following administration of the TNFα inhibitor.

In one embodiment, the invention provides a method of determining the efficacy of a TNFα inhibitor for maintaining remission of Crohn's disease in a subject comprising determining a Crohn's Disease Activity Index (CDAI) score of a patient population having Crohn's disease and who was administered the TNFα inhibitor, to wherein a CDAI score of less than 150 maintained in at least about 49% of the patient population indicates that the TNFα inhibitor is an effective TNFα inhibitor for the treatment of Crohn's disease in a subject, including, but not limited to, maintenance of remission of Crohn's disease. In one embodiment, the method further comprises administering the effective TNFα inhibitor to a subject to maintain remission of Crohn's disease. The invention provides a method of maintaining remission of Crohn's disease in a subject comprising administering an effective amount of a TNFα inhibitor to the subject such that remission of Crohn's disease is maintained, wherein the effective amount of the TNFα inhibitor was previously identified as maintaining a CDAI score of less than 150 in at least about 49% of a patient population having Crohn's disease.

In one embodiment the invention provides a method of determining the efficacy of a TNFα inhibitor for maintaining remission of Crohn's disease in a subject comprising determining a Crohn's Disease Activity Index (CDAI) score of a patient population having Crohn's disease and who was administered more than one maintenance dose the TNFα inhibitor, wherein a CDAI score of less than 150 maintained in at least about 47% of the patient population indicates that the TNFα inhibitor is an effective TNFα inhibitor for the treatment of Crohn's disease in a subject.

In one embodiment the invention provides a method of determining the efficacy of a TNFα inhibitor for maintaining remission of Crohn's disease in a subject comprising determining a Crohn's Disease Activity Index (CDAI) score of a patient population having Crohn's disease and who was administered a human TNFα antibody, or antigen-binding portion thereof, wherein a CDAI score of less than 150 maintained in at least about 32% of the patient population indicates that the TNFα inhibitor is an effective TNFα inhibitor for the treatment of Crohn's disease in a subject.

In one embodiment, a CDAI score of less than 150 maintained in at least about 32% of the patient population indicates that the TNFα inhibitor is an effective TNFα inhibitor for the treatment of Crohn's disease in a subject. In one embodiment, a CDAI score of less than 150 maintained in at least about 36% of the patient population indicates that the TNFα inhibitor is an effective TNFα inhibitor for the treatment of Crohn's disease in a subject. In one embodiment, a CDAI score of less than 150 maintained in at least about 40% of the patient population indicates that the TNFα inhibitor is an effective TNFα inhibitor for the treatment of Crohn's disease in a subject. In one embodiment, a CDAI score of less than 150 maintained in at least about 41% of the patient population indicates that the TNFα inhibitor is an effective TNFα inhibitor for the treatment of Crohn's disease in a subject. In one embodiment, a CDAI score of less than 150 maintained in at least about 46% of the patient population indicates that the TNFα inhibitor is an effective TNFα inhibitor for the treatment of Crohn's disease in a subject. In one embodiment, a CDAI score of less than 150 maintained in at least about 47% of the patient population indicates that the TNFα inhibitor is an effective TNFα inhibitor for the treatment of Crohn's disease in a subject. In one embodiment, a CDAI score of less than 150 maintained in at least about 50% of the patient population indicates that the TNFα inhibitor is an effective TNFα inhibitor for the treatment of Crohn's disease in a subject. In one embodiment, a CDAI score of less than 150 maintained in at least about 60% of the patient population indicates that the TNFα inhibitor is an effective TNFα inhibitor for the treatment of Crohn's disease in a subject. In one embodiment, a CDAI score of less than 150 maintained in at least about 67% of the patient population indicates that the TNFα inhibitor is an effective TNFα inhibitor for the treatment of Crohn's disease in a subject. In one embodiment, a CDAI score of less than 150 maintained in at least about 70% of the patient population indicates that the TNFα inhibitor is an effective TNFα inhibitor for the treatment of Crohn's disease in a subject. In one embodiment, a CDAI score of less than 150 maintained in at least about 74% of the patient population indicates that the TNFα inhibitor is an effective TNFα inhibitor for the treatment of Crohn's disease in a subject. In one embodiment, a CDAI score of less than 150 maintained in at least about 79% of the patient population indicates that the TNFα inhibitor is an effective TNFα inhibitor for the treatment of Crohn's disease in a subject. In one embodiment, a CDAI score of less than 150 maintained in at least about 80% of the patient population indicates that the TNFα inhibitor is an effective TNFα inhibitor for the treatment of Crohn's disease in a subject. In one embodiment, a CDAI score of less than 150 maintained in at least about 83% of the patient population indicates that the TNFα inhibitor is an effective TNFα inhibitor for the treatment of Crohn's disease in a subject. In one embodiment, a CDAI score of less than 150 maintained in at least about 84% of the patient population indicates that the TNFα inhibitor is an effective TNFα inhibitor for the treatment of Crohn's disease in a subject. In one embodiment, a CDAI score of less than 150 maintained in at least about 85% of the patient population indicates that the TNFα inhibitor is an effective TNFα inhibitor for the treatment of Crohn's disease in a subject. In one embodiment, a CDAI score of less than 150 maintained in at least about 90% of the patient population indicates that the TNFα inhibitor is an effective TNFα inhibitor for the treatment of Crohn's disease in a subject. In one embodiment, a CDAI score of less than 150 maintained in at least about 94% of the patient population indicates that the TNFα inhibitor is an effective TNFα inhibitor for the treatment of Crohn's disease in a subject. In one embodiment, the CDAI improvement and/or maintenance of remission indicated by a CDAI score of less than 150 is found in at least about 32% of the patient population. In another embodiment, at least about 36%. In another embodiment, at least to about 40%. In another embodiment, at least about 41%. In another embodiment, at least about 46%. In another embodiment, at least about 47%. In another embodiment, at least about 50%. In another embodiment, at least about 60%. In another embodiment, at least about 67%. In another embodiment, at least about 70%. In another embodiment, at least about 74%. In another embodiment, at least about 79%. In another embodiment, at least about 80%. In another embodiment, at least about 83%. In another embodiment, at least about 84%. In another embodiment, at least about 85%. In another embodiment, at least about 90%. In another embodiment, at least about 94%.

Numbers intermediate to the above recited percentages, e.g., 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, as well as all other numbers recited herein, are also intended to be part of this invention. Ranges of values using a combination of any of the above recited values as upper and/or lower limits are intended to be included in the scope of the invention. For example, in one embodiment a CDAI score of less than 150 maintained in at least between 47% and 79% of the patient population indicates that the TNFα inhibitor is an effective TNFα inhibitor for the treatment of Crohn's disease in a subject.

In one embodiment, the invention provides a method of determining the efficacy of TNFα inhibitor, e.g., a human TNFα antibody, or antigen-binding portion thereof, for achieving a clinical response in Crohn's disease in a subject comprising determining a Crohn's Disease Activity Index (CDAI) score of a patient population having Crohn's disease and who was administered the TNFα inhibitor, wherein a decrease of at least 100 in the CDAI score of at least about 47% of the patient population indicates that the TNFα inhibitor is an effective TNFα inhibitor for achieving a clinical response in Crohn's disease in a subject. In one embodiment, the method further comprises administering the effective TNFα inhibitor to a subject to achieve a clinical response in Crohn's disease. The invention also provides a method of achieving a clinical response in Crohn's disease in a subject comprising administering an effective amount of a TNFα inhibitor to the subject such that a clinical response in Crohn's disease is achieved, wherein the effective amount of the TNFα inhibitor was previously identified as decreasing a CDAI score by at least 100 in at least about 47% of a patient population having Crohn's disease.

The invention also provides a method of determining the efficacy of a TNFα inhibitor, e.g., a human TNFα antibody, or antigen-binding portion thereof, for maintaining remission of Crohn's disease comprising determining a Crohn's Disease Activity Index (CDAI) score of a patient population having Crohn's disease and who to was administered the TNFα inhibitor, wherein A 100, i.e., a decrease of at least 100 in the CDAI score, in at least about 41% of the patient population indicates that the TNFα inhibitor is effective for maintaining remission of Crohn's disease.

In one embodiment, the invention provides a method of determining the efficacy of a TNFα inhibitor for achieving a clinical response in Crohn's disease in a subject comprising determining a Crohn's Disease Activity Index (CDAI) score of a patient population having Crohn's disease and who was administered the TNFα inhibitor, wherein a decrease of at least 100 in the CDAI score of at least about 47% of the patient population indicates that the TNFα inhibitor is an effective TNFα inhibitor for achieving a clinical response in Crohn's disease in a subject.

In one embodiment, the invention provides a method of determining the efficacy of a TNFα inhibitor for achieving a clinical response in Crohn's disease in a subject who has not received infliximab comprising determining a Crohn's Disease Activity Index (CDAI) score of a patient population having Crohn's disease and who was administered the TNFα inhibitor, wherein a decrease of at least 100 in the CDAI score of at least about 41% of the patient population indicates that the TNFα inhibitor is an effective TNFα inhibitor for achieving a clinical response in Crohn's disease in a subject in a subject who has net received infliximab.

In one embodiment, a decrease of at least 100 in the CDAI score of at least about 41% of the patient population indicates that the TNFα inhibitor is an effective TNFα inhibitor for achieving a clinical response in Crohn's disease in a subject. In one embodiment, a decrease of at least 100 in the CDAI score of at least about 48% of the patient population indicates that the TNFα inhibitor is an effective TNFα inhibitor for achieving a clinical response in Crohn's disease in a subject. In one embodiment, a decrease of at least 100 in the CDAI score of at least about 50% of the patient population indicates that the TNFα inhibitor is an effective TNFα inhibitor for achieving a clinical response in Crohn's disease in a subject. In one embodiment, a decrease of at least 100 in the CDAI score of at least about 52% of the patient population indicates that the TNFα inhibitor is an effective TNFα inhibitor for achieving a clinical response in Crohn's disease in a subject. In one embodiment, a decrease of at least 100 in the CDAI score of at least about 60% of the patient population indicates that the TNFα inhibitor is an effective TNFα inhibitor for achieving a clinical response in Crohn's disease in a subject. In one embodiment, a decrease of at least 100 in the CDAI score of at least about 64% of the patient population indicates that the TNFα inhibitor is an effective TNFα inhibitor for achieving a clinical response in Crohn's disease in a subject. In one embodiment, a decrease of at least 100 in the CDAI score of at least about 67% of the patient population indicates that the TNFα inhibitor is an effective TNFα inhibitor for achieving a clinical response in Crohn's disease in a subject. In one embodiment, a decrease of at least 100 in the CDAI score of at least about 70% of the patient population indicates that the TNFα inhibitor is an effective TNFα inhibitor for achieving a clinical response in Crohn's disease in a subject. In one embodiment, a decrease of at least 100 in the CDAI score of at least about 74% of the patient population indicates that the TNFα inhibitor is an effective TNFα inhibitor for achieving a clinical response in Crohn's disease in a subject. In one embodiment, a decrease of at least 100 in the CDAI score of at least about 79% of the patient population indicates that the TNFα inhibitor is an effective TNFα inhibitor for achieving a clinical response in Crohn's disease in a subject. In one embodiment, a decrease of at least 100 in the CDAI score of at least about 80% of the patient population indicates that the TNFα inhibitor is an effective TNFα inhibitor for achieving a clinical response in Crohn's disease in a subject. In one embodiment, a decrease of at least 100 in the CDAI score of at least about 83% of the patient population indicates that the TNFα inhibitor is an effective TNFα inhibitor for achieving a clinical response in Crohn's disease in a subject. In one embodiment, a decrease of at least 100 in the CDAI score of at least about 84% of the patient population indicates that the TNFα inhibitor is an effective TNFα inhibitor for achieving a clinical response in Crohn's disease in a subject. In one embodiment, a decrease of at least 100 in the CDAI score of at least about 85% of the patient population indicates that the TNFα inhibitor is an effective TNFα inhibitor for achieving a clinical response in Crohn's disease in a subject. In one embodiment, a decrease of at least 100 in the CDAI score of at least about 89% of the patient population indicates that the TNFα inhibitor is an effective TNFα inhibitor for achieving a clinical response in Crohn's disease in a subject. In one embodiment, a decrease of at least 100 in the CDAI score of at least about 90% of the patient population indicates that the TNFα inhibitor is an effective TNFα inhibitor for achieving a clinical response in Crohn's disease in a subject. In one embodiment, a decrease of at least 100 in the CDAI score of at least about 94% of the patient population indicates that the TNFα inhibitor is an effective TNFα inhibitor for achieving a clinical response in Crohn's disease in a subject. In one embodiment, a decrease of at least 100 in the CDAI score of at least about 41% of the patient population indicates that the TNFα inhibitor is an effective TNFα inhibitor for achieving a clinical response in Crohn's disease in a subject. In another embodiment, the percentage is at least about 45%. In another embodiment, at least about 48%. In another embodiment, at least about 50%. In another embodiment, at least about 52%. In another embodiment, at least about 60%. In another embodiment, at least about 64%. In another embodiment, at least about 67%. In another embodiment, at least about 70%. In another embodiment, at least about 74%. In another embodiment, at least about 79%. In another embodiment, at least about 80%. In another embodiment, at least about 89%. In another embodiment, at least about 90%. In another embodiment, at least about 95%. In another embodiment, to at least about 100%.

Numbers intermediate to the above recited percentages, e.g., 41%, 42%, 43%, 44%, 45%, 46%, 47%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93% are also intended to be part of this invention. Ranges of values using a combination of any of the above recited values as upper and/or lower limits are intended to be included in the scope of the invention. For example in one embodiment a decrease of at least 100 in the CDAI score of at least between 52%-74% of the patient population indicates that the TNFα inhibitor is an effective TNFα inhibitor for achieving a clinical response in Crohn's disease in a subject.

In one embodiment, the invention provides a method of determining the efficacy of a TNFα inhibitor, e.g., a human TNFα antibody, or antigen-binding portion thereof, for achieving a clinical response in Crohn's disease in a subject comprising determining a Crohn's Disease Activity Index (CDAI) score of a patient population having Crohn's disease and who was administered the TNFα inhibitor, wherein a decrease of at least 70 in the CDAI score of at least about 43% of the patient population indicates that the TNFα inhibitor is an effective TNFα inhibitor for achieving a clinical response in Crohn's disease in a subject. In one embodiment, the invention further comprises administering the effective TNFα inhibitor to a subject in need thereof. In one embodiment, the invention provides a method of achieving a clinical response in Crohn's disease in a subject comprising administering an effective amount of a TNFα inhibitor to the subject such that a clinical response in Crohn's disease is achieved, wherein the effective amount of the TNFα inhibitor was previously identified as decreasing a CDAI score by at least 70 in at least about 43% of a patient population having Crohn's disease.

In one embodiment, a decrease of at least 70 in the CDAI score of at least about 49% of the patient population indicates that the TNFα inhibitor is an effective TNFα inhibitor for achieving a clinical response in Crohn's disease in a subject. In one embodiment, a decrease of at least 70 in the CDAI score of at least about 50% of the patient population indicates that the TNFα inhibitor is an effective TNFα inhibitor for achieving a clinical response in Crohn's disease in a subject. In one embodiment, a decrease of at least 70 in the CDAI score of at least about 54% of the patient population indicates that the TNFα inhibitor is an effective TNFα inhibitor for achieving a clinical response in Crohn's disease in a subject. In one embodiment, a decrease of at least 70 in the CDAI score of at least about 56% of the patient population indicates that the TNFα inhibitor is an effective TNFα inhibitor for achieving a clinical response in Crohn's disease in a subject. In one embodiment, a decrease of at least 70 in the CDAI score of at least about 58% of the patient population indicates that the TNFα inhibitor is an effective TNFα inhibitor for achieving a clinical response in Crohn's disease in a subject. In one embodiment, a decrease of at least 70 in the CDAI score of at least about 60% of the patient population indicates that the TNFα inhibitor is an effective TNFα inhibitor for achieving a clinical response in Crohn's disease in a subject. In one embodiment, a decrease of at least 70 in the CDAI score of at least about 70% of the patient population indicates that the TNFα inhibitor is an effective TNFα inhibitor for achieving a clinical response in Crohn's disease in a subject. In one embodiment, a decrease of at least 70 in the CDAI score of at least about 80% of the patient population indicates that the TNFα inhibitor is an effective TNFα inhibitor for achieving a clinical response in Crohn's disease in a subject. In one embodiment, a decrease of at least 70 in the CDAI score of at least about 90% of the patient population indicates that the TNFα inhibitor is an effective TNFα inhibitor for achieving a clinical response in Crohn's disease in a subject. In one embodiment, a decrease of at least 70 in the CDAI score of at least about 43% of the patient population indicates that the TNFα inhibitor is an effective TNFα inhibitor for achieving a clinical response in Crohn's disease in a subject. In another embodiment, the percent of the patient population is about 49%. In another embodiment, at least about 49%. In another embodiment, at least about 54%. In another embodiment, at least about 56%. In another embodiment, at least about 58%.

Numbers intermediate to the above recited percentages, e.g., 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% are also intended to be part of this invention. Ranges of values using a combination of any of the above recited values as upper and/or lower limits are intended to be included in the scope of the invention. For example in one embodiment a decrease of at least 70 in the CDAI score of at least between 56%-70% of the patient population indicates that the TNFα inhibitor is an effective TNFα inhibitor for achieving a clinical response in Crohn's disease in a subject.

In one embodiment, the efficacy of a TNFα inhibitor is determined by determining the QOL or HRQL of a patient population having Crohn's disease. The QOL or HQRL may be determined by one or more patient reported outcomes (PRO) scales or scores, including, but not limited to, an Inflammatory Bowel Disease Questionnaire (IBDQ) score, Short Form Health Surveys (e.g., SF-36 or SF-12), FACIT-f (fatigue), the Zung Depression score and a Visual Analog Score system for assessing abdominal pain. Health related quality of life (HRQL) may be measured by a variety of types of measurement techniques. For example, in some embodiments, generic quality of life instruments (e.g. SF-36), disease-specific quality of life instruments (e.g. HAQ), to and/or health utility instruments (e.g. Health Utilities Index Mark 3 [HUI3]) can be used.

The invention also provides a method of determining the efficacy of a TNFα inhibitor for treating Crohn's disease using an Inflammatory Bowel Disease Questionnaire (IBDQ) score of a patient population having Crohn's disease. The IBDQ, a 32-item questionnaire, was developed to provide a measure of health status for clinical trials in inflammatory bowel disease (see Guyatt et al. Gastroenterology. 1989; 96:804-10, the contents of which are expressly incorporated herein by reference). It evaluates quality of life with respect to bowel function (e.g. loose stools and abdominal pain), systemic symptoms (fatigue and altered sleep pattern), social function (work attendance and the need to cancel social events) and emotional status (angry, depressed, or irritable). The score ranges from 32 to 224, with higher scores indicating a better quality of life. In one embodiment, the methods further comprises administering the effective TNFα inhibitor to a subject having Crohn's disease.

In one embodiment, the invention provides a method of determining the efficacy of a TNFα inhibitor to maintain remission of Crohn's disease in a subject comprising determining an Inflammatory Bowel Disease Questionnaire (IBDQ) score of a patient population having Crohn's disease who was administered the TNFα inhibitor, wherein an IBDQ score greater than 170 in at least about 74% of the patient population indicates that the TNFα inhibitor is an effective TNFα inhibitor for maintaining remission of Crohn's disease in a subject.

The invention includes a method of maintaining remission of Crohn's disease in a subject comprising administering an effective amount of a TNFα inhibitor to the subject, such that remission of Crohn's disease is maintained, wherein the effective amount of the TNFα inhibitor was previously identified as maintaining an IBDQ score greater than 170 in at least about 74% of a patient population having Crohn's disease.

In one embodiment, an IBDQ score greater than 170 in at least about 40% of the patient population indicates that the TNFα inhibitor is an effective TNFα inhibitor for maintaining remission of Crohn's disease in a subject. In one embodiment, an IBDQ score greater than 170 in at least about 50% of the patient population indicates that the TNFα inhibitor is an effective TNFα inhibitor for maintaining remission of Crohn's disease in a subject. In one embodiment, an IBDQ score greater than 170 in at least about 60% of the patient population indicates that the TNFα inhibitor is an effective TNFα inhibitor for maintaining remission of Crohn's disease in a subject. In one embodiment, an IBDQ score greater than 170 in at least about 70% of the patient population indicates that the TNFα inhibitor is an effective TNFα inhibitor for maintaining remission of Crohn's disease in a subject. In one embodiment, an IBDQ score greater than 170 in at least about 80% of the patient population indicates that the TNFα inhibitor is an effective TNFα inhibitor for maintaining remission of Crohn's disease in a subject. In one embodiment, an IBDQ score greater than 170 in at least about 83% of the patient population indicates that the TNFα inhibitor is an effective TNFα inhibitor for maintaining remission of Crohn's disease in a subject. In one embodiment, an IBDQ score greater than 170 in at least about 74% indicates that the TNFα inhibitor is an effective TNFα inhibitor for maintaining remission of Crohn's disease in a subject. In another embodiment, at least about 76%. In another embodiment, at least about 78%. In another embodiment, at least about 80%, In another embodiment, at least about 83%.

Numbers intermediate to the above recited percentages, e.g., 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82% are also intended to be part of this invention. Ranges of values using a combination of any of the above recited values as upper and/or lower limits are intended to be included in the scope of the invention. For example, in one embodiment an IBDQ score greater than 170 in at least between about 50%-83% of the patient population indicates that the TNFα inhibitor is an effective TNFα inhibitor for maintaining remission of Crohn's disease in a subject.

FACIT-fatigue measures the impact of disease or other conditions on levels of fatigue experienced by a patient. Studies showed that patients regard oppressive fatigue as a major determinant of their overall HRQL (Kirwan et al 2005). The FACIT-Fatigue was used to assess fatigue in patients enrolled in a number of the studies described below. The FACIT-Fatigue scale includes 13 specific items linked with fatigue: fatigue, weakness, listlessness, tiredness, trouble with starting things, trouble with finishing things, energy, activity, sleep, eating, help doing activities, frustration, and social activities. FACIT-Fatigue scores range from 0 to 52, with higher scores representing less fatigue. The instrument has been validated for the general population and for patients with autoimmune and other types of diseases. For example, the MCID (minimum clinically important difference) for FACIT-Fatigue in rheumatoid arthritis was determined to be at least a 4-point change from baseline (Cella et al, 2005).

In some embodiments of the present invention, the invention includes using a FACIT-F score of a patient population who has been administered a TNFα inhibitor to determine whether the TNFα inhibitor is effective at treating Crohn's disease or psoriasis. In some embodiments, for example, FACIT-F may be administered at baseline, at 1 or 2 time points during a study, and at the end of a study. The questionnaire asks each patient the following: “Below is a list of statements that other people with your illness have said are important. By circling one (1) number per line, please indicate how true each statement has been for you during the past 7 days.” The questions in the questionnaire used to determine the FACIT score includes the following, where each patient indicated the appropriate number (0-4): I feel fatigued; I feel weak all over; I feel listless (“washed out”); I feel tired; I have trouble starting things because I am tired; I have trouble finishing things because I am tired; I have energy; I am able to do my usual activities; I need to sleep during the day; I am too tired to eat; I need help doing my usual activities; I am frustrated by being too tired to do the things I want to do; I have to limit my social activity because I am tired. FACIT-F scores range from 0-52, with higher scores representing less fatigue. FACIT-F changes of ≧4 are considered clinically meaningful. The results of improvement in fatigue levels in patients receiving adalimumab can be compared to those receiving placebo, adalimumab in different amounts or dosing regimens, adalimumab concurrently with other therapies or other therapies in lieu of adalimumab.

The invention also provides a method of determining the efficacy of a TNFα inhibitor for treating Crohn's disease using an Visual Analog Score system for assessing the abdominal pain experienced by a patient population having Crohn's disease. Visual Analog Score systems may be used to assess patient pain for a variety of diseases, conditions and locations, to record pain intensity, increases in pain and/or pain relief. With a Visual Analog Score system, the patient reports the amount of pain experienced at the time of assessment and pain reports can be compared to previous or subsequent reports to estimate changes in patient-perceived pain.

The invention includes a method of determining the efficacy of a human TNFα antibody, or antigen-binding portion thereof, for achieving a clinical response in Crohn's disease in a subject comprising determining the percentage complete fistula closing of a patient population having Crohn's disease and who was administered the human TNFα antibody, or antigen-binding portion thereof, wherein complete fistula closing observed in at least about 28% of the patient population indicates that the human TNFα antibody, or antigen-binding portion thereof, is an effective human TNFα antibody, or antigen-binding portion thereof, for achieving a clinical response in Crohn's disease in a subject. In one embodiment, the percentage complete fistula closing observed in a patient population is at least about 28%. In anther embodiment, at least about 30%. In another embodiment, at least about 33%. In another embodiment, at least about 35%. In anther embodiment, at least about 37% of the patient population.

Methods of the invention relating to determining efficacy, i.e., determining whether a TNFα inhibitor is an effective TNFα inhibitor, may also be applied to specific patient populations within the overall patient population who together have specific, common characteristics, i.e., a subpopulation. For example, the patient population may comprise patients on concomitant immunosuppressant (IMM) treatment with the TNFα inhibitor. In another example, the patient population may comprises patients not on concomitant IMM treatment.

In addition, while the above methods are described in terms of patient populations, methods of efficacy described herein may also be applied to individual subjects. For example, a method for determining efficacy may comprise determining whether a subject who is in remission from Crohn's disease, and who is on a dosage regimen comprising a human TNFα antibody, is able to maintain a CDAI of less than 150 to determining if the human TNFα antibody is an effective human TNFα antibody.

In one embodiment, if the subject is able to maintain remission of Crohn's disease for at least about 26 weeks, then the human TNFα antibody is effective at maintaining remission of Crohn's disease.

The Examples and discoveries described herein are representative of a TNFα inhibitor, i.e., adalimumab, which is effective for treating Crohn's disease, including inducing and maintaining remission of Crohn's. As such, the studies and results described in the Examples section herein may be used as a guideline for determining the efficacy of a TNFα inhibitor, i.e., whether a TNFα inhibitor is an effective TNFα inhibitor for the treatment of Crohn's disease. In one embodiment, methods of determining efficacy described herein may be used to determine whether a TNFα inhibitor is bioequivalent to another TNFα inhibitor.

In one embodiment, the article of manufacture of the invention comprises instructions regarding how to determine the efficacy of the TNF inhibitor for the treatment of Crohn's disease.

The Short Form 36 (SF-36) Health Survey is a generic, patient reported, health-related quality of life (HRQOL) measurement instrument, typically with 8 domains and two summary scores for physical and mental health—physical functioning, role-physical, bodily pain, general health, vitality, social functioning, role-emotional, and mental health. These 8 domains were aggregated into Physical Component Summary (PCS) and Mental Component Summary (MCS) scores. The SF-36 has a scale of 0-100, with higher scores indicating better health-related quality of life. Minimum clinically important differences (MCID) are defined as improvements of 5-10 points in the individual domains scores and 2.5-5 points in the PCS and MCS (Kosinski M, et al. Arthritis Rheum 2000:43:1478-87). The SF-12 Health Survey is a subset of items from the SF-36 survey.

In some embodiments, HRQOL domains are measured at baseline, and after 12, 26, 42, 52, 76, and 104 weeks of therapy. In some of these analyses, mean scores and mean changes in each HRQOL domain are reported, as well as the PCS and MCS, at a variety of intervals, for example, at Weeks 12 and 104. Criteria-based interpretation was used to understand the meaning of differences in PCS scores for work loss and resource use and content-based interpretation for specific SF-36 items. In some embodiments, order to interpret the results of surveys using the SF-36 PRO, criteria-based and content-based interpretations are used to gain a further understanding of differences in SF-36 Physical Component Summary (PCS) scores (Ware J E, Kosinski M. SF-36 Physical & Mental Health Summary Scales: A Manual for Users of Version 1. 2nd ed. Lincoln, R1: QualityMetric Incorporated, November 2002). Content-based interpretation can be based on analyses of the content of individual SF-36 items within the survey for the general US population, such as “Does your health limit you in walking one block?” Criteria-based interpretation can be based on external criteria such as predicting job loss due to health problems and is also based on US population norms for PCS scores.

The Zung Depression scale is a PRO measure designed to assess levels of depression. The measure is self-administered and assesses the four common characteristics of depression: pervasive effect, physiological equivalents, other disturbances and psychomotor activities. One taking the survey will usually respond to twenty statements, either positively or negatively worded (typically ten of each), by stating how much of the time that they feel that way. The patient will rate each statement with a numerical value of one to four, with four equaling the strongest agreement with the statement, i.e. “most of the time”. Scores range from 25 to 100, with 25-49 as the normal range, 50-59 as mildly depressed, 60-69 as moderately depressed and 70 and above as severely depressed (Zung, W., Arch Gen Psych (1965) 12:63-70).

### VII. Additional Embodiments

The articles of manufacture described herein for use in the treatment of psoriasis and Crohn's disease may further contain labels or package inserts that contain additional information pertaining to the benefits and risks of TNFα antagonist (e.g., TNFα antibody) administration.

For example, in one embodiment, the package insert may contain clinical information indicating that the risks to fetuses and young infants in pregnant and nursing women exposed to adalimumab has not been established. In another embodiment, the label may indicate that the safety and effectiveness of adalimumab in pediatric patients have not been established. In another embodiment, the label may report the results of clinical studies indicating that the frequency of serious infection and malignancy among treated subjects over age 65 was higher than for those under age 65. In another embodiment, the label or package insert may indicate that doses of up to 10 mg/kg have been administered to patients in clinical trials without evidence of dose-limiting toxicities.

In another embodiment, the label or package insert may contain information pertaining to the storage and handling of the TNFα antibody (e.g., adalimumab). For example, the package insert may indicate that the antibody is supplied in prefilled syringes or pens, e.g., containing 1 mL with a fixed 27 gauge ½ inch needle, providing 40 mg (0.8 mL) of the antibody. In another embodiment, the label or package insert may indicate that the prefilled syringes or pens should be refrigerated (e.g., at 2 to 8° C.; 36 to 46° F.). In another embodiment, the label or package insert may indicate that the prefilled syringes or pens should not be frozen. In another embodiment, the label or package insert may indicate that the prefilled syringes or pens should be protected from exposure to light. In another embodiment, the label or package insert may indicate that a puncture-resistant container for disposal of the needles and syringes should be used.

In other embodiments, the label or package insert may contain information pertaining to patient counseling. In one embodiment, the package insert indicates that clinicians should advise patients of the potential benefits and risks of the TNFα antibody (e.g., adalimumab). In one embodiment, the package insert indicates that clinicians should instruct their patients to read the Medication Guide before starting antibody therapy and to reread each time the prescription is renewed. In one embodiment, the package insert indicates that clinicians should inform patients that the antibody may lower the ability of their immune system to fight infections (e.g., including tuberculosis and reactivation of hepatitis B virus infections. In one embodiment, the package insert indicates that clinicians should counsel patients about the risk of lymphoma and other malignancies. In one embodiment, the package insert indicates that clinicians should advise patients to seek immediate medical attention if they experience any symptoms of severe allergic reactions. In one embodiment, the package insert indicates that clinicians should advise latex-sensitive patients that the needle cap of the prefilled syringe contains latex. In one embodiment, the package insert indicates that clinicians should advise patients to report any signs of new or worsening medical conditions such as heart disease, neurological disease, or autoimmune disorders. In one embodiment, the package insert indicates that clinicians should advise patients to report any symptoms suggestive of a cytopenia such as bruising, bleeding, or persistent fever.

In related embodiments, the label or package insert contains a Medication Guide for patients who are to be treated with the TNFα antibody (e.g., adalimumab). For example, the Guide may contain information and questions for patients pertaining to the benefits and risks of treatment. In some embodiments, the package insert may inform patients to notify their clinician before starting treatment if they have one or more of the following: any kind of infection, even if it is very minor (such as an open sore); are being treated for an infection; have signs of an infection, such as a fever, cough, or flu-like symptoms; have warm, red, or painful skin; get a lot of infections or have infections that keep coming back; have or had hepatitis B infection; have TB, or have been in close contact with someone who has TB; have lived in an area where TB or histoplasmosis is common; were born in, lived in, or traveled to countries where there is more risk for getting TB; take the medicine Kineret (anakinra); are scheduled to have major surgery; have any numbness or tingling or have a disease that affects your nervous system such as multiple sclerosis or Guillain-Barré syndrome; have had heart failure or other heart conditions; have recently received or are scheduled to receive a vaccine; are allergic to rubber or latex; are allergic to the TNFα antibody (adalimumab) or to any of its ingredients (e.g., sodium phosphate, sodium citrate, citric acid, mannitol and polysorbate 80); if you are pregnant, planning to become pregnant or breastfeeding. In other embodiments, the package insert may inform patients to inform their clinician of all medicines that they are taking, including prescription and nonprescription medicines, vitamins and herbal supplements, especially Kineret (anakinra).

In other related embodiments, the Medication Guide may advise patients to notify their clinician after starting treatment if they develop one or more of the following: any kind of infection; a fever; fatigue; a cough; flu-like symptoms; warm, red, or painful skin; open sores; weight loss; loss of body fat and muscle (wasting); poor appetite; joint pain; nervous system problems (e.g., numbness or tingling, problems with vision, weakness in arms or legs, dizziness); blood problems (e.g., a fever that does not go away, bruising or bleeding very easily, or looking very pale); new heart failure or worsening of heart failure (e.g., shortness of breath, swelling of ankles or feet, sudden weight gain); immune reactions including lupus-like syndrome (e.g., chest discomfort or pain that does not go away, shortness of breath, joint pain, or a rash on cheeks or arms that gets worse in the sun).

In further embodiments, the label or package insert may contain instructions on injection techniques. For example, in one embodiment the label or package insert may contain instructions for patients or clinicians for administration of a prefilled pen containing the TNFα antibody (e.g., adalimumab) that includes one or more of the following steps:

(1) Setting up for an injection
find a clean flat surface
Take one dose tray containing a Pen from the refrigerator. 1 alcohol prep (swab), 1 cotton ball or gauze pad. Do not use if the seals on top and bottom of carton are broken or missing. Do not use a Pen that has been frozen or if it has been left in direct sunlight. Do not use the pen if the expiration date on the label has passes.

2) Choosing and preparing an injection site
Wash your hands well
Choose a site on the front of your thighs or your stomach area (abdomen).
If you choose your abdomen, you should avoid the area 2 inches around your belly button (navel).
Choose a different site each time you give yourself an injection. Each new injection should be given at least one inch from a site you used before. Never inject into areas where the skin is tender, bruised, red or hard or where you have scars or stretch marks.
If you have psoriasis, you should try not to inject directly into any raised, thick, red or scaly skin patches or lesions.
You may find it helpful to keep notes on the location of your injection sites.
Wipe the site where dose is to be injected with an alcohol prep (swab), using a circular motion. Do not touch this area again until you are ready to inject.

3) How to prepare your dose for injection with a Pen
Hold the Pen with the gray cap pointing up. Check the solution through the windows on the side of the Pen to make sure the liquid is clear and colorless. Do not use a Pen if the liquid is cloudy or discolored or has flakes or particles in it. Do not use if frozen.
Turn the Pen over and hold the Pen with the gray cap pointed down. Check to make sure that the amount of liquid in the Pen is the same or close to the fill line seen through the window. The fill line represents a full dose of the product. The top of the liquid may be curved. If the Pen does not have the full amount of liquid, do not use that pen. Call your pharmacist.

4) Injecting the dose
Hold the Pen with one hand. With your other hand, remove the gray cap (1) and discard cap. Pull the cap straight off. Do not twist the cap. Check that the small gray needle cover of the syringe has come off with the cap. After removal, the needle cover is held in the cap. Do not touch the needle. The white needle sleeve, which covers the needle, can now be seen. Do not put the gray cap (1) back on or you may damage the needle. Do not drop or crush the product as it contains a glass syringe that may break.
Remove the plum colored safety cap (2) to expose the plum colored push button at the top. Pull the cap straight off. Do not twist the cap. The Pen is now ready to use. Please note that the Pen is activated after removing the plum colored safety cap 2 and that pressing the button under the plum colored safety cap 2 will release the medicine from the syringe. Do not press the button until you are ready to inject HUMIRA. Do not put the colored cap (2) back on the pen as this could cause medicine to come out of the syringe.
Hold the Pen so that the window can be seen.
With your free hand, gently squeeze an area of the cleaned skin at the injection site. You will inject into this raised area of skin.
Place the end of the Pen straight (a 90° angle) and flat against the raised area of skin. Place the Pen so that it will not inject the needle into your fingers that are holding the raised skin.
With your first (index) finger, press the button to begin the injection. You may also use your thumb to press the plum colored button to begin the injection. Try not to cover the window. You will hear a ‘click’ when you press the button, which means the start of the injection. Keep pressing the button and continue to hold the Pen against the raised skin until all of the medicine is injected. This can take up to 10 seconds. It is important to keep holding the pen against the raised skin of your injection site for the whole time.
You will know that the injection has finished when marker appears fully in the window view and stops moving.
When the injection is finished, pull the Pen from the skin. The needle sleeve will move to cover the needle tip.
Press a cotton ball or gauze pad over the injection site and hold it for 10 seconds. Do not rub the injection site. You may have slight bleeding. This is normal.
Dispose of the Pen right away into your special sharps container.
Do not try to touch the needle. The needle sleeve is there to prevent you from touching the needle.

In another embodiment the label or package insert may contain instructions for patients or clinicians for administration of a prefilled syringe containing the TNFα antibody (e.g., adalimumab) that includes one or more of the following steps described above except that steps 3 and 4 are as follows:

3) How to Prepare Your Tnfα Antibody Dose for Injection with a Prefilled Syringe
Hold the syringe upright with the needle facing down. Check to make sure that the amount of liquid in the syringe is the same or close to the 0.8 mL line shown on the prefilled syringe. The top of the liquid may be curved. If the syringe does not have the correct amount of liquid, do not use that syringe. Call your pharmacist.
Remove the needle cover taking care not to touch the needle with your fingers or allow it to touch any surface.
Turn the syringe so the needle is facing up and slowly push the plunger in to push the air in the syringe out through the needle. If a small drop of liquid comes out of the needle that is okay. Do not shake the syringe.
   4) Injecting TNFα antibody
With your other hand, gently squeeze an area of the cleaned area of skin and hold it firmly. You will inject into this raised area of skin. Hold the syringe like a pencil at about a 45° angle to the skin.
With a quick, short, “dart-like” motion, push the needle into the skin.
After the needle is in, let go of the skin. Pull back slightly on the plunger. If blood appears in the syringe it means that you have entered a blood vessel. Do not inject TNFα antibody. Pull the needle out of the skin and repeat the steps to choose and clean a new injection site. Do not use the same syringe. Dispose of it in your special sharps container. If no blood appears, slowly push the plunger all the way in until all of the TNFα antibody is injected.
When the syringe is empty, remove the needle from the skin keeping it at the same angle it was when it was pushed into the skin.
Press a cotton ball or gauze pad over the injection site and hold it for 10 seconds. Do not rub the injection site. You may have slight bleeding. This is normal.
Dispose of the syringe right away into your special sharps container.

In further related embodiments, the Medication Guide or package insert may further include instructions on how to dispose of used needles and syringes. In one embodiment, Medication Guide or package insert instructs the patient or clinician to follow any special state or local laws regarding the disposal of needles and syringes. In one embodiment, the Medication Guide or package insert instructs the patient or clinician not throw the needle or syringe in the household trash or recycle trash. In another embodiment, the Medication Guide or package insert instructs the patient or clinician to Place the used needles and syringes in a container made specially for disposing of used syringes and needles (called a “Sharps” container), or a hard plastic container with a screw-on cap or metal container with a plastic lid labeled “Used Syringes”. In another embodiment, the Medication Guide or package insert instructs the patient or clinician to always keep the disposal container out of the reach of children. In another embodiment, the Medication Guide or package insert instructs the patient that when the container is about two-thirds full, tape the cap or lid down so it does not come off and dispose of it as instructed by your doctor, nurse or pharmacist. In still another embodiment, the Medication Guide or package insert instructs the patient that used alcohol pads, cotton swabs may be placed in the trash, unless otherwise instructed by your doctor, nurse or pharmacist. In still another embodiment, the Medication Guide or package insert instructs the patient that the dose tray and cover may be placed in your recycle trash.

The present invention is further illustrated by the following examples which should not be construed as limiting in any way.

### EXAMPLES

### Example 1

### Psoriasis Patients Treated Continuously with Adalimumab: Efficacy and Safety Results from Month 12 to 18

### Objective:

Adalimumab is a fully human, IgG₁ monoclonal antibody specific for tumor necrosis factor, a pivotal cytokine in the pathogenesis of psoriasis. This analysis was designed to determine the long-term (up to 18 months) efficacy and safety of adalimumab in patients with moderate to severe psoriasis.

### Methods:

REVEAL (Randomized Controlled EValuation of Adalimumab Every Other Week Dosing in Moderate to Severe Psoriasis TriAL) was a 52 week, randomized, double-blind, placebo-controlled, Phase III clinical trial of adalimumab in 1,212 patients for the treatment of moderate to severe chronic plaque psoriasis. Patients who completed REVEAL could subsequently enroll in an open-label extension (OLE), during which continuous adalimumab therapy was administered. The experience during the first 6 months of OLE for the subset of patients who had received continuous adalimumab for 12 months in REVEAL was summarized based on interim analyses conducted in May 2007. PASI responses were analyzed relative to the baseline of REVEAL for the intention-to-treat population, defined as patients who received continuous adalimumab dosing in the 52-week REVEAL, completed REVEAL, and received ≧1 dose of adalimumab in OLE.

Main inclusion criteria for REVEAL were: clinical diagnosis of psoriasis for ≧6 months; affected body surface area (BSA)≧10%; PASI≧12; and a Physician's Global Assessment (PGA) of at least “Moderate”. Main exclusion criteria was the previous use of systemic anti-TNF therapy. The study measured PASI 75, 90 and 100 response rates at weeks 0, 12, and 24 of the OLE period. PGA scores were assessed at weeks 0, 12 and 24 of the OLE period. Laboratory parameters and adverse events were also recorded.

The REVEAL study had two independent primary endpoints. The first endpoint was the proportion of patients achieving 75 percent improvement in skin clearance after 16 weeks. The second endpoint was the proportion of patients who lost adequate response through week 52 after stopping treatment with HUMIRA at week 33. Signs of psoriasis were evaluated using the Psoriasis Area and Severity Index (PASI), among other measures. Patients receiving adalimumab who achieved at least a PASI 75 response at week 16 continued to receive adalimumab on an open-label basis. At week 33, the 490 patients who maintained PASI 75 were randomized to receive placebo or continue receiving adalimumab. At week 52, the start of the OLE period, patients randomized at week 33, as well as patients originally in the placebo group at week 0, were able to enter the OLE period. Patients in the OLE period received 40 mg adalimumab EOW.

### Results:

PASI 75 response scores were measures at the end of weeks 4, 8, 12, 16 and 24 during the double-blind phase of the study. PASI 75, 90 and 100 response rates were also measured at weeks 0, 12 and 24 of the OLE (weeks 52, 64 and 76 after the start of to REVEAL). PGA scores were assessed at the same timepoints during OLE. Results are shown below in Tables 1-1, 1-2 and 1-3.

TABLE 1-1
PASI 75 Response: Week 0 to 24

| | Placebo† | Adalimumab‡ |
|---|---|---|
| Week 4 | 1.3% | 18.9%* |
| Week 8 | 3.0% | 54.1%* |
| Week 12 | 4.8% | 67.7%* |
| Week 16 | 6.5% | 71.0%* |
| Week 24 | | 70.3%** |

*p < 0.001 adalimumab vs placebo;
†n = 398;
‡n = 814
**Pooling of efficacy outcomes from Period B (wks. 16 to 33) and OLE ITT: NRI

TABLE 1-2
PASI Response Rates During the First 24 Weeks of OLE

| | PASI 75 | PASI 90 | PASI 100 |
|---|---|---|---|
| Week 0 | 85% | 84% | 87% |
| Week 12 | 59% | 60% | 63% |
| Week 24 | 35% | 33% | 34% |

n = 233;
ITT: Last observation carried forward (LOCF).

TABLE 1-3
PGA Scores of “Clear” or “Minimal” During
the First 24 Weeks of the OLE

| | PGA “Clear” or “Minimal” |
|---|---|
| Week 0 | 70% |
| Week 12 | 71% |
| Week 24 | 74% |

n = 233; ITT: LOCF

TABLE 1-4
Adverse Events During the First 24 Weeks of OLE

| Adverse Events (AEs) | Patients (%) |
|---|---|
| Any AE | 47 |
| AEs leading to withdrawal | 1 |
| Infectious AEs | 18 |
| Serious AE* | 3 |
| Serious infectious AE | 1 |
| Malignancy† | 1 |
| Lymphoma | 0 |
| Non-melanoma skin cancer | 0 |
| AEs occurring ≧5% of patients | |
| Nasopharyngitis | 5 |

*Serious AEs included a bile duct stone, coronary artery disease in a patient with a history of coronary artery bypass graft, myocardial infarction (MI) in a patient with a history of 2 previous MIs, renal cell carcinoma, gastroenteritis, and staphylococcal infection.
†Renal cell carcinoma and prostate cancer.

A total of 233 patients were included in the intention-to-treat population. Their PASI 75/90/100 response rates were 85%/59%/35% after 12 months of continuous adalimumab dosing, and 87%/63%/34% after 18 months. Six patients (2.6%) experienced serious adverse events, and 2 patients (0.9%) reported serious infections (between Months 12-18).

PGA scores of “clear” or “minimal were reported for 74% of patients.

### Conclusion:

Patients who had received adalimumab for 12 months experienced sustained improvement when continued through 18 months. Adalimumab was safe and well-tolerated for up to 18 months of treatment. Long-term adalimumab treatment of patients with moderate to severe psoriasis was associated with sustained and substantial degrees of improvement and a low risk of serious infections, which suggests a favorable benefit/risk balance.

### Example 2

### Methotrexate-treated Psoriasis Patients Transitioning to Adalimumab: Efficacy and Safety Outcomes

### Objective:

Adalimumab is a fully human, IgG₁ monoclonal antibody that inhibits TNF, a pivotal cytokine in the pathogenesis of psoriasis. This analysis was conducted to determine the efficacy and safety of transitioning methotrexate-treated psoriasis patients to adalimumab.

### Methods:

CHAMPION was a 16-week, Phase III, active- and placebo-controlled trial in which patients with moderate to severe chronic plaque psoriasis were randomized to receive placebo, methotrexate, or adalimumab. Patients who completed CHAMPION could subsequently enroll in an open-label extension (OLE) study, during which patients received adalimumab 40 mg every other week. Patient experience associated with transitioning from methotrexate to adalimumab was summarized based on interim analyses conducted in May 2007. PASI responses were analyzed relative to baseline of CHAMPION for the intention-to-treat population, defined as patients who were randomized to methotrexate in the 16-week CHAMPION, completed CHAMPION, and received ≧1 dose of adalimumab in the OLE.

### Results:

At week 16 of CHAMPION, PASI response rates were evaluated from placebo, methotrexate and adalimumab patients. Results are shown in Table 3-1.

TABLE 3-1
PASI Response Rates at Week 16 of CHAMPION

| | Placebo (N = 53) | MTX (N = 110) | ADA (N = 108) |
|---|---|---|---|
| PASI 50 | 30.2% | 61.8% | 88.0%*† |
| PASI 75 | 18.9% | 35.5% | 79.6%*† |
| PASI 90 | 11.3% | 13.6% | 51.9%*† |
| PASI 100 | 1.9% | 7.3% | 16.7%‡§ |

*p < 0.001 vs. placebo;
†p < 0.001 vs. MTX;
‡p < 0.01 vs. placebo;
§p < 0.05 vs. MTX.
Analysis: ITT, nonresponder imputation (NRI).
ITT population defined as population randomized in CHAMPION.
Saurat JH, et al. Br J Dermatol. 2007; DOI: 10.1111/j.1365-2133.2007.08315.x.a.

The intention-to-treat population comprised 95 patients. These patients received a mean methotrexate dose of 19.1 mg in the penultimate week of CHAMPION. Their PASI 75/90/100 response rates were 28%/14%/5% prior to starting adalimumab, 75%/47%/18% after 12 weeks of adalimumab therapy and 73%/53%/32% after 24 weeks of adalimumab therapy. No cases of rebound were observed, and no patients experienced serious infections during the first 24 weeks of the OLE.

Adverse events are presented in Table 3-2.

TABLE 3-2
Adverse Events through Week 24 of the OLE (N = 95)

| Adverse Events (AE) | Percentage of Patients |
|---|---|
| Any AE | 47 |
| AEs leading to withdrawal | 1 |
| Infectious AEs | 15 |
| Serious AE* | 5 |
| Serious infectious AE | 0 |
| Rebound psoriasis | 0 |
| Any malignancy | 0 |
| AEs occurring ≧5% of patients | |
| Nasopharyngitis | 6 |
| Injection-site reaction | 5 |

*Serious AEs during the OLE included pneumothorax, neuralgia, avascular necrosis of hip, spontaneous abortion, and motorbike accident.

### Conclusion:

The transition of patients with psoriasis from methotrexate to adalimumab was safe and well-tolerated (no rebound psoriasis observed when adalimumab was started 1 week after MTX was discontinued), with low risk of serious infections. Adalimumab therapy led to substantial skin improvements in these transitioning patients and was found to be more efficacious than MTX for treatment of moderate to severe chronic plaque psoriasis.

### Example 3

### Impact of Adalimumab (HUMIRA®) on Patient-Reported Outcomes Among Patients With Fistulizing Crohn's Disease in the CHARM Trail

Introduction: Fistulas occur in 17 to 43 percent of patients with Crohn's disease (CD) and fistulizing disease is associated with worsening quality of life. Complete and sustained fistula closure has been associated with adalimumab (ADA) therapy in the CHARM trial, a Phase III randomized, double-blinded, placebo-controlled assessment of ADA in maintaining clinical remission in patients with CD.¹

### Aims & Methods:

The impact of ADA maintenance therapy on CD-specific health-related quality of life among randomized patients with draining fistulas observed at screening visits and at baseline (BL) of the CHARM trial was assessed. Inflammatory Bowel Disease Bowel Disease Questionnaire (IBDQ) evaluations were conducted at BL and at Weeks 4, 12, 26, and 56 of the CHARM trial. IBDQ scores over time between groups receiving ADA, 40 mg every other week (EOW) or 40 mg every week (EW), or placebo (PBO), were compared using Analysis of Covariance. The proportions of patients achieving ≧16-point improvement in IBDQ from BL, the minimum clinically meaningful improvement, were compared using Chi-square.

### Results:

Of 117 patients with fistulizing CD who entered the study, 75 had IBDQ measurements after Week 4 and were followed through Week 56. Statistically significant and clinically meaningful results observed with ADA maintenance were sustained through Week 56. Mean changes in IBDQ scores from BL and the proportion of patients achieving ≧16-point gain in IBDQ from BL at Week 56 are presented (Table 3-1).

TABLE 3.1
Change From Baseline IBDQ and % of Patients With
≧16-Point Total IBDQ Gain at Week 56

| | | 40 mg ADA | 40 mg ADA |
|---|---|---|---|
| | PBO | EOW | EW |
| N | 26 | 21 | 28 |
| Mean Improvement in | 21.6 | 43.5* | 46.8* |
| Total IBDQ | | | |
| ≧16-point IBDQ | 46% | 76%* | 86%** |
| Improvement | | | |

*p < 0.05,
**p < 0.01, both vs. PBO.

### Conclusion:

Adalimumab maintenance therapy is associated with sustained and clinically meaningful improvement of CD-specific quality of life among patients with fistulizing Crohn's Disease as measured by the IBDQ.

### REFERENCES

1. Colombel J F, et al. Gastroenterology. 2007; 132:52-65.

### Example 4

### Impact of Adalimumab (HUMIRA®) on Patient-Reported Outcomes

### Introduction:

Adalimumab (ADA) is approved for the treatment of adults with moderate to severe Crohn's disease (CD) in the US. The CHARM trial was a 56-week randomized, double-blinded, placebo-controlled, Phase III study of ADA efficacy and safety in maintaining clinical remission in patients with moderately to severely active CD.¹

### Aims & Methods:

The effect of ADA maintenance on patient-reported outcomes (PROs) was assessed among Randomized Responders, patients (pts) with a decrease ≧70 points from baseline (BL) CDAI score at Week (Wk) 4 in CHARM. All patients received an induction regimen of open-label (OL) ADA 80 mg at BL (Wk 0), and 40 mg ADA at Wk 2. At Wk 4, patients were stratified by response and randomized to a maintenance regimen (40 mg ADA every other week or every week), or placebo. After Wk 12, patients with response loss or flare were allowed to switch to OL ADA. PROs (SF-36, IBDQ, FACIT-Fatigue, and Zung Depression) collected at Wks 0, 4, 12, 26, and 56, were evaluated for differences between the effects of ADA JO and ADA induction and maintenance (IM) therapy among Randomized Responders.

### Results:

In all, 499 responders were randomized at Wk 4 (JO, n=170; 1M, n=329). At Wk 0, patients had an impaired health-related quality of life (HRQL) as indicated by PROs. Patients improved across all 4 PROs during the induction phase (Wk 4 vs. BL). PRO measures from Wk 12 through Wk 56 showed a significantly improved quality of life for patients in the IM group compared with pateints in the IO group. SF-36 PCS was better for the IM group from Wk 12 through Wk 56 (all p<0.05 vs. JO), and MCS was also better for the IM group at Wks 26 and 56 (both p≦0.05). The improvement in disease specific IBDQ scores was very significant and sustained for the IM group (p<0.01 vs. IO, Wks 12-56) throughout the study. FACIT-Fatigue scores showed fatigue symptoms were lessened in the IM group (p<0.01 vs. JO at Wks 12-56). Reductions in Zung Depression scores showed significantly reduced depression symptoms in the IM group (p<0.01 vs. JO at Wks 12-56).

### Conclusion:

Adalimumab maintenance therapy resulted in significant and sustained improvements in CD-specific (IBDQ) and general (SF-36 PCS and MCS) HRQL measurements, compared with induction only therapy. Substantial and sustained benefits were observed for fatigue and depression outcomes in CD patients receiving ADA maintenance therapy.

### REFERENCES

1. Colombel J F, et al. Gastroenterology. 2007; 132(1):52-65.

### Example 5

### Impact of Steroid Discontinuation on Health Care Resource Utilization in Crohn's Disease

### Introduction:

Although effective for some patients (pts) with Crohn's Disease (CD), steroids have been associated with considerable adverse events with longer durations of therapy. The economic impact of steroid use (SU) in CD could be significant. The objective of this study was to determine whether discontinuation of SU among patients with CD lowers CD-related health care costs.

### Aims & Methods:

A total of 9,811 pts with CD-related SU were selected from an Integrated Healthcare Information Services (IHCIS) managed care database (1999-2005). The index date was defined as the first date of CD-related steroids use (CD documented with the past 30 days) after 3-month continuous insurance coverage eligibility. Patients with SU 60-90 days after the index date were steroid maintainers (SM). All others were steroid discontinuers (S-). Health care costs (total and CD-related) of S- and SM were evaluated over 3-months and compared descriptively. The same groups were then controlled for baseline (BL) characteristics using a regression-adjusted model and compared. Costs were inflation adjusted to year 2005 in US dollars ($).

### Results:

Of the 9,811 CD patients selected from IHClS, 5,614 were S- and 4,197 were SM. Mean age was 43.1 years for both groups. There were 44.4% (S-) and 46.7% (SM) males. Both groups had similar prior total and CD-related health care costs. In the follow-up period, both total and CD-related costs were statistically significantly lower for S- when compared with SM (both p<0.01). Similarly, total and CD-related regression-adjusted costs were also statistically significantly lower for S- when compared with SM (both p<0.01). Significantly lower CD-related health care costs were observed for all health care cost components of S-, including inpatient, outpatient, and prescription drug costs.

TABLE 5-1
Health Care Costs by Steroid Use Status

| | | | Regression-Adjusted | |
|---|---|---|---|---|
| | Descriptive Costs | | Costs | |
| | 3-month | 3-month CD- | 3-month | 3-month CD- |
| US $ | total cost | related cost | total cost | related cost |
| SM (n = 4,197) | 10,786 | 5,270 | 9,513 | 5,352 |
| S— (n = 5,614) | 7,759* | 3,275* | 8,477* | 3,209* |

*p < 0.01,
S— vs. SM for respective cost groups.

### Conclusions:

Steroid discontinuation was statistically significantly associated with lower total and CD-related health care costs. These results demonstrated continuous steroid use may have significant cost implications for society and third-party payers. Therapies that help patients taper off steroids may be associated with potential cost savings.

### Example 6

### Steroid Use and Patient Reported Outcomes: Analysis of Charm Trial Data

### Introduction:

A significant proportion of patients with moderate to severe Crohn's disease (CD) receiving adalimumab (ADA) in the CHARM study were able to discontinue steroid therapy. Long-term steroid therapy has been associated with decreased quality of life (QOL) reported by patients with ulcerative colitis. To date, however, the impact of long-term QOL for patients with Crohn's disease (CD) has not been widely reported.

### Aims & Methods:

Patient reported outcomes (PRO) were compared among 233 patients who had any CD-related steroid use at baseline (Week 0) from the Phase III randomized, double-blinded, placebo-controlled CHARM trial. PRO assessments, including Inflammatory Bowel Disease Questionnaire (IBDQ), SF-36 physical (PCS) and mental (MCS) components, Zung depression scale, FACIT-fatigue, and a VAS abdominal pain index were collected at weeks 0, 4, 12, 26, and 56. Week 12 PRO data for patients with CD who were steroid discontinuers vs. those who were steroid maintainers were compared in this analysis. Mean scores for all PRO measures were compared using t-tests.

### Results

Steroid discontinuers reported statistically better QOL vs. steroid maintainers as measured by IBDQ (169.98 vs. 158.34, p=0.040), SF-36 MCS (p=0.008), depression (p=0.016), and abdominal pain (p=0.029) (Table 6-1).

TABLE 6-1
Mean PRO at Week 12 by Steroid Discontinuation Status

| | |
|---|---|
| Steroid | Steroid |
| Maintainers | Discontinuers |

| PRO | N | Mean | N | Mean | p-value |
|---|---|---|---|---|---|
| IBDQ* | 165 | 158.34 | 46 | 169.98 | 0.040 |
| SF-36 MCS* | 126 | 45.26 | 36 | 50.28 | 0.008 |
| Depression^{†} | 165 | 48.87 | 45 | 43.83 | 0.016 |
| Fatigue* | 165 | 34.28 | 46 | 37.72 | 0.286 |
| Abdominal | 165 | 40.60 | 46 | 34.06 | 0.029 |
| Pain^{†} | | | | | |

*Higher scores correlate to an improved QOL.
^{†}Lower scores correlate to an improved QOL.

### Conclusions:

Patients with Crohn's disease who discontinued steroids reported statistically significant better quality of life than patients who maintained steroid treatment at 12 weeks. Therapies that help CD patients taper off steroids may improve their quality of life.

### Example 7

### The Effects of Adalimumab Maintenance Therapy on Health-Related Quality of Life of Patients with Crohn's Disease: Patient-Reported Outcomes of the CHARM Trial

The following examples expands on the studies referred to above in Examples 3, 4, and 6.

### Overview:

The objective of the study was to evaluate the impact of adalimumab maintenance therapy on health-related quality of life (HRQOL) in patients with moderate to severe Crohn's disease. In a phase III, randomized, double-blind clinical trial (CHARM) of moderate to severe Crohn's disease patients, HRQOL outcomes were compared between the adalimumab maintenance treatment groups (every other week and weekly injection) and the adalimumab induction-only group. The Zung Self-Rating Depression Scale, FACIT-Fatigue, visual analog pain scales, IBDQ, and SF-36 were analyzed for the 499 randomized responders (a decrease of ≧70 points from baseline in the CDAI Crohn's Disease Activity Index) at baseline and weeks 4, 12, 26, and 56.

Results showed that the HRQOL of participants in CHARM was substantially impaired at baseline. Following a 4-week adalimumab induction therapy, patients experienced statistically significant improvements in all HRQOL measures during weeks 0-4 (P<0.0001). Compared with patients who were assigned to placebo beginning at week 4, patients who received adalimumab 40 mg every other week reported less depression (P<0.01), fewer fatigue symptoms (P<0.001), greater improvements in the IBDQ (P<0.05), greater normalized SF-36 Physical Component Summary scores (P<0.05), and less abdominal pain (P<0.05) from weeks 12-56. They also reported greater normalized SF-36 Mental Component Summary scores at week 56 (P<0.05). Patients who received adalimumab 40 mg weekly reported less depression and fewer fatigue symptoms at week 56, as well as greater improvement in IBDQ and less abdominal pain from weeks 12-56 (all P<0.05 vs. placebo). In conclusion, adalimumab maintenance therapy provided sustained improvements in health-related quality of life for patients with moderate to severe Crohn's disease through week 56.

### Methods:

### Patients

The study design and methods have been described in detail elsewhere (9). Briefly, male and female patients between the ages of 18 and 75 were eligible to participate in the study if they had a diagnosis of Crohn's disease for at least 4 months prior to the beginning of the study (confirmed by endoscopic or radiologic evaluation), and a CDAI score between 220 and 450 at the time of enrollment. Patients with a history of having received TNF-antagonist therapy were permitted to enroll if they had discontinued therapy at least 12 weeks before study entry. Other concurrent treatments for Crohn's disease were allowed, including stable dosages (for ≧4 weeks before screening) of azathioprine, 6-mercaptopurine, methotrexate, 5-aminosalicylates, sulfasalazine, oral mesalamine, and Crohn's disease-related antibiotics, as well as stable dosages (for ≧2 weeks before screening) of prednisone (≦30 mg/day) or budesonide (≦9 mg/day) (9). Additional inclusion and exclusion criteria are detailed in the primary clinical efficacy and safety report of this trial (9).

### Protocol and Assignment

Patients were screened for two weeks prior to their baseline assessments. At the baseline visit (week 0), all 854 patients received open-label adalimumab 80-mg subcutaneous injection, followed by a 40-mg dose at week 2. At week 4, patients who experienced a decrease in CDAI scores of 70 or more points from baseline were considered responders. A total of 778 patients were randomized to one of three treatment arms: adalimumab 40 mg every other week (eow), adalimumab 40 mg weekly, and induction-only (adalimumab induction therapy at weeks 0 and 2, followed by placebo injections through the remainder of their participation in the study) (9). A total of 76 patients withdrew prior to randomization (9).

Among the 499 responders at week 4, 170 were randomized to the adalimumab induction-only arm (placebo from week 4 up to week 56, or last visit), 172 to the adalimumab 40-mg eow arm, and 157 to the adalimumab 40-mg weekly arm. Patients who experienced flares (increase in CDAI of ≧70 points vs. week 4 and a CDAI score of >220) or sustained non response (did not achieve a decrease in CDAI ≧70 points from. baseline) were allowed to switch to open-label adalimumab 40 mg eow at or after week to 12. The dosing frequency could be increased to weekly with recurrent flare or continued non response (9).

### Participant Flow

These analyses evaluated the 499 week-4 randomized responders. As previously reported, these 499 patients represented the randomized responder sample assessed in the predefined primary efficacy endpoint analysis (9).

### Data Collection and Health-Related Quality of Life Instruments

Patients were asked to complete the Zung Self-Rating Depression Scale, FACIT-F scale, IBDQ, SF-36, and a VAS of abdominal pain at baseline (week 0) and at weeks 4, 12, 26, and 56. Patients who discontinued prior to the end of the trial were asked to complete the surveys at the early termination visit (before Week 56).

The Zung Self-Rating Depression Scale consists of 20 self-rated questions, whose individual scores are aggregated into a single score. The scale has been validated in previous studies as a measurement of general depression (10). Scores range from 20-80, with greater scores indicating more severe symptoms of depression. A score <50 is considered to be within the normal range, whereas 50-59 represents mild depression, 60-69 is considered moderate depression, and a score >70 indicates severe depression (11).

The FACIT-F scale was first used to measure the severity of fatigue in anemia (12). The instrument consists of 13 items, each of which is scored on a 5 point Likert scale of fatigue symptoms. The total scores range from 0 to 52, with lower scores reflecting greater fatigue. A change of 3-4 points is considered clinically meaningful (13). The instrument has demonstrated good validity and responsiveness to change (12).

The IBDQ was designed specifically to assess overall HRQOL in patients with IBD and consists of 32 questions. The validity, reliability, and responsiveness of IBDQ scores to changes in Crohn's disease status have been previously established (14). Moreover, the IBDQ is the most commonly used disease-specific HRQOL instrument for IBD studies (15). Its subscales cover four general aspects of HRQOL: bowel-related symptoms (loose stools, abdominal pain); systemic complaints (fatigue, sleep pattern); social function (ability to attend work and social events); and emotional status (anger, depression, irritability). IBDQ scores range from 32-224, with greater scores indicating better HRQOL (16). An increase of ≧16 points in the total score represents a clinically meaningful improvement, and an overall score of ≧170 points correlates with remission (14, 17). In addition, the IBDQ scores correlate with CDAI scores and clinical symptoms of Crohn's disease. Therefore, IBDQ is a particularly valuable measure of HRQOL in IBD patients (14).

The SF-36, a commonly used generic instrument for HRQOL measurement which has been validated in multiple countries (18,19), consists of 36 questions spanning 8 domains: physical function, role limitations—physical, role limitations—emotional, vitality, general health perceptions, pain, social function, and mental health. The SF-36 can be summarized using two component scores: the Physical Component Summary (PCS) and the Mental Component Summary (MCS). Each summary score incorporates all eight domain scores, but with different weights for each domain. Greater summary scores indicate better HRQOL (7). Based on standard-scoring algorithms, the scores presented in this study were standardized and normalized to those of the general U.S. population. Therefore, each scale has a population mean score of 50 and standard deviation of 10. Normalized SF-36 scores permit the comparison of HRQOL of patients with Crohn's disease with the HRQOL of the general U.S. population. The standard scoring algorithm has been shown to be equivalent to country-specific scoring algorithms. It can be used in multinational studies (19). A change of 3-5 points in the MCS or PCS scale is generally accepted as a meaningful change (20). The SF-36 has been used to measure HRQOL of patients with Crohn's disease in clinical trials (8).

Finally, participants in CHARM were also asked to rate their abdominal pain using a VAS. VAS scales are frequently used in HRQOL analyses as a reliable measurement of pain symptoms, and have been validated in several studies (11). Patients were presented a line scale indicating “no abdominal pain” (far left) to “extreme pain” (far right). The indicated value on this continuum was then translated into a number between 0-100, with greater scores indicating greater pain severity and intensity.

### Statistical Analysis

Baseline characteristics, including patient demographics and disease information, of the week-4 responders (N=499) by treatment arm (induction-only, adalimumab 40-mg eow maintenance therapy, and adalimumab 40-mg weekly maintenance therapy) were evaluated using descriptive statistical techniques. Mean and standard deviations were reported for continuous variables, and number of patients and percentage of total patients were reported for categorical variables.

Differences in HRQOL measures between the two adalimumab maintenance groups and the induction-only group were assessed using least-squares-means from the analysis of covariance (ANCOVA). In this ANCOVA method, corresponding HRQOL baseline values and previous use of TNF antagonist (no, yes) were controlled for as covariates in the analysis. Patients who discontinued their randomized therapies early or who switched to open-label adalimumab 40-mg eow had their last observations carried forward (LOCF) through the remaining follow-up visits. For each HRQOL measure, only patients with values at either baseline or week 4, as well as one or more follow-up visit at or after week 12, were included in the analyses.

### Results:

### Baseline Characteristics

A total of 854 patients enrolled in the trial, 778 of whom were randomized at week 4 (9). Of these, 499 were classified as responders and were included in the current HRQOL analyses presented here. Because of differences in data availability, sample sizes varied slightly for each HRQOL assessment. The baseline characteristics were similar between treatment groups (Table 7-1). No statistically significant differences were observed among the treatment groups in the HRQOL scores at baseline or at the end of the induction phase (week 4)

At baseline the trial participants were experiencing substantial impairment in HRQOL (Table 7-2). The mean Zung Depression score was 55.5 points overall, indicating mild depression. Patients' mean FACIT-F scores were 22.9 points at baseline, approximately half of the average score for the U.S. general population, and similar to scores of patients with cancer-related anemia (12). The baseline mean IBDQ total score was approximately 125 points, 45 points below the IBDQ score of 170 that correlates with clinical remission. For the SF-36, both the mean normalized PCS and MCS scores for the induction-only and adalimumab maintenance-therapy groups were around 17 points below those of the general U.S. population averages, indicating a substantial burden of the disease (21). The mean VAS-reported abdominal pain was 57.5 points at baseline, reflecting moderate pain.

### Response to Induction Treatment

Following administration of the open-label induction regimen, there were statistically significant improvements for all HRQL measures at week 4 vs. week 0 (p<0.0001 for all scales, weeks 0-4) (Table 7-2).

### Depression

At the end of the induction phase, the mean values for reported depression symptoms on the Zung Depression scale had decreased from the baseline value of 55.5 points to 46.0 points, which corresponds to an improvement from mild depression to the normal range (P<0.0001) (Table 7-2). Following the induction phase, the mean values for symptoms of depression increased (worsened) for patients assigned to placebo, but slightly decreased (improved) for the patients who continued treatment with adalimumab 40-mg eow and 40-mg weekly (Table 7-3). The scores for the adalimumab eow maintenance to group reflected statistically significantly greater improvements in depression compared with induction-only patients at all time points after week 4 (P<0.01), while the group who received weekly injections exhibited statistically significantly greater improvements in comparison to placebo-treated patients only at week 56 (P<0.05).

### Fatigue

Following induction therapy, the FACIT-F scores increased dramatically from the baseline value (net change, 11.9 points, p<0.0001), greater than the minimal clinical meaningful change (3-4 points) (13) (Table 7-2). Following week 4, patients who received placebo reported worsening fatigue at subsequent visits (Table 7-4). In contrast, from weeks 4-12, the adalimumab 40-mg eow maintenance-therapy group further improved, and maintained a statistically significantly greater difference vs. the induction-only group from week 12 onwards (P<0.001). Similar results were observed in the patients assigned to weekly treatment with adalimumab at week 56 (P<0.05).

### Inflammatory Bowel Disease Questionnaire

During the induction phase, the mean IBDQ total score increased nearly 45 points across groups, indicating a substantial improvement in HRQOL (P<0.0001, week 4 vs. baseline) (Table 7-2). During subsequent visits, the mean IBDQ total scores of the two adalimumab maintenance-therapy groups trended to further increase, gaining approximately 5 points from weeks 4-56, while the patients assigned to placebo experienced deteriorating scores. By week 56, the mean IBDQ total scores for both adalimumab maintenance groups were higher than the 170-point threshold associated with remission, whereas placebo-treated patients had an average score >7 points below the threshold. The differences in mean IBDQ total score between the adalimumab-maintenance and induction-only groups were statistically significantly different at all visits after week 4 (P<0.001 for adalimumab eow and P<0.05 for adalimumab weekly). At week 56, patients in the adalimumab 40-mg eow arm had a mean IBDQ score approximately 14 points greater than that of the induction-only group (P<0.001). Consistent with the observations in IBDQ total scores, with one exception (Systemic Function sub-scale at week 12), there were statistically significant differences for the IBDQ subscale scores between the adalimumab maintenance groups and the induction-only group at every visit following week 4 (P<0.05 for both groups vs. induction-only group) (Table 7-5).

### Short Form 36 Health Survey

During induction therapy (baseline to week 4), the mean PCS score for all groups increased 8 points (about one standard deviation), indicating a clinically important improvement in physical well-being (P<0.0001, week 4 vs. baseline) (Table 2). After week 4, the PCS score for the adalimumab 40-mg eow maintenance group was significantly greater than the score of the induction-only group at all subsequent visits (P<0.05) (Table 7-6).

During induction therapy (baseline to week 4), the mean MCS scores improved approximately 10 points (slightly less than one standard deviation) (P<0.0001, week 4 vs. baseline) (Table 7-2). During subsequent visits, the scores for adalimumab maintenance groups trended upwards, while scores in patients assigned to placebo the induction-only group decreased slightly. For the eow maintenance group, the difference was statistically significant at week 56 (P<0.05) (Table 7-6).

An examination of results across all of the SF-36 sub domains showed that patients in the adalimumab eow maintenance group experienced greater improvements in HRQOL than patients in the induction-only group. The differences in role—physical (P≦0.001), bodily pain (P<0.05), social functioning (P<0.01), and mental health (P<0.05) were all statistically significant at week 56.

### Abdominal Pain Scores

During induction the mean self-reported abdominal pain score was nearly halved for the randomized responder sample (P<0.001, week 4 vs. baseline) (Table 7-2). After induction, the mean abdominal pain score reported by the induction-only group increased. Patients in the induction-only group reported more than a 4.6-point increase from weeks 4-12, and nearly 8 points from weeks 4-56 (Table 7-7). In contrast, reported pain scores decreased (representing improvements) for both maintenance groups from weeks 4-12, after which the scores remained fairly stable for the remainder of the study. The differences between the induction-only group and both adalimumab maintenance-therapy groups were statistically significant for all time points after week 4 (P<0.001 for eow, and P<0.05 for weekly) (Table 7-7).

### Discussion

In this analysis of the CHARM trial, baseline HRQOL measures indicated impairment despite the use of traditional Crohn's disease therapies. At baseline/screening, the percentages of all patients were receiving corticosteroids, immunosuppressive agents, or 5-aminosalicylates were 42%, 48%, and 41%, respectively. In addition, nearly half had previously used anti-TNF therapies.

Following induction, patients in all treatment groups had achieved statistically significant improvements across all HRQOL measures (baseline to week 4). After week 4, patients in the adalimumab eow and weekly maintenance-therapy groups to demonstrated sustained improvements in all HRQOL measures employed, vs. patients receiving induction-only therapy. Despite having realized similar gains during the induction phase, induction-only patients generally fared worse over the full course of the study. By contrast, HRQOL scores for adalimumab eow or weekly maintenance-therapy patients continued to improve, demonstrating sustainability of the early benefits achieved during the 4-week induction phase. The findings of the Zung Depression, FACIT-F, IBDQ, SF-36, and VAS abdominal pain scales all similarly point toward the ability of adalimumab maintenance therapy to sustain patients' improvements in HRQOL.

The results presented may be overly conservative because of the statistical treatment of missing observations using LOCF. Despite the clear benefits of maintenance therapy, the LOCF method may underestimate the worsening of outcomes for the induction-only group, which had more drop-outs and greater rates of switching to open-label adalimumab therapy than the other two groups. Since dropping out of the study altogether or switching to open-label therapy are likely to be correlated with worsening symptoms, the scores for the induction-only group may have further decreased if these patients had continued to receive placebo. Patients in the induction-only group would likely have had much worse HRQOL scores, on average, if they had the same dropout and switching rates as the other treatment groups. Therefore, the estimated benefits of maintenance therapy observed in the trial may potentially be considered conservative.

In CHARM, the baseline average depression symptoms scores were characteristic of mild depression. With adalimumab maintenance therapy, patients were able to improve to the normal range during the induction phase and continued to improve through week 56. These improvements in depressive symptoms may reflect the efficacy of adalimumab in achieving clinical response, clinical remission, fistula closure, and steroid tapering (9).

Baseline FACIT-F scores for CHARM patients were similar to those of patients with cancer-related anemia (12). Patients in all three groups experienced statistically improvements in fatigue symptoms during the induction phase (decrease of approximately 12 points). Following induction, the maintenance groups further improved or remained the same, while the induction-only group worsened slightly at every subsequent visit. The adalimumab eow maintenance group exhibited fewer fatigue symptoms vs. the induction-only group at all subsequent visits, and for both maintenance groups, these differences were statistically significant at week 56. Further, the differences between the eow group and the induction-only group were consistently more than 4 points from weeks 12-56, a clinically meaningful difference (13, 21). Moreover, this degree of change has been shown to be associated with an increase of hemoglobin concentrations in patients (22).

In conclusion, adalimumab maintenance therapy provided sustained benefits across varied aspects of HRQOL for patients with moderate to severe Crohn's disease. Notably, adalimumab maintenance therapy has been demonstrated to improve symptoms of depression and fatigue symptoms—a first for a biologic in the treatment of Crohn's disease. Physicians should take these into account as well when identifying their comprehensive treatment plan.

TABLE 7-1
Baseline (Week 0) Demographics and Clinical Characteristics

| | | Adalimumab | Adalimumab |
|---|---|---|---|
| | Adalimumab | Maintenance | Maintenance |
| | Induction- | Therapy | Therapy |
| | Only | (40 mg eow) | (40 mg weekly) |
| Number of patients | N = 170 | N = 172 | N = 157 |
| randomized at week 4 | | | |
| Male, n (%) | 65 (38%) | 61 (36%) | 62 (39%) |
| Mean age, yrs (SD) | 36.9 (11.9) | 36.4 (11.1) | 36.9 (11.8) |
| Mean body weight, kg | 70.4 (18.8) | 70.4 (17.3) | 69.8 (17.5) |
| (SD) | | | |
| Involved intestinal | | | |
| areas, n (%) | | | |
| Ileum | 113 (66%) | 125 (73%) | 119 (76%) |
| Colon | 130 (76%) | 126 (73%) | 119 (76%) |
| Ileum and colon | 75 (44%) | 81 (47%) | 84 (54%) |
| Gastro-duodenum | 8 (5%) | 12 (7%) | 10 (6%) |
| Other | 26 (15%) | 21 (12%) | 21 (15%) |
| Enterocutaneous or | 29 (17%) | 14 (8%) | 22 (14%) |
| perianal fistula at both | | | |
| screening and baseline, | | | |
| n (%) | | | |
| Mean CDAI score | 321.1 (67.1) | 315.7 (61.5) | 312.6 (58.3) |
| (SD) | | | |
| CRP concentration | 85 (50%) | 76 (44%) | 75 (48%) |
| ≧1.0 mg/dL, n (%) | | | |
| Previous TNF- | 83 (49%) | 86 (50%) | 75 (48%) |
| antagonist exposure, | | | |
| n (%) | | | |
| Concomitant | | | |
| medication, N (%) | | | |
| Any corticosteroid | 66 (39%) | 58 (34%) | 74 (47%) |
| Prednisone | 57 (34%) | 41 (24%) | 57 (36%) |
| Budesonide | 11 (6%) | 17 (10%) | 17 (11%) |
| Any | 83 (49%) | 77 (45%) | 79 (50%) |
| immunosuppressive | | | |
| agent | | | |
| Azathioprine | 60 (35%) | 52 (30%) | 44 (28%) |
| 6-Mercaptopurine | 11 (6%) | 8 (5%) | 18 (11%) |
| Methotrexate | 13 (8%) | 17 (10%) | 19 (12%) |
| 5-Aminosalicylates | 78 (46%) | 66 (38%) | 61 (69%) |
| Current smoker, N (%) | 63 (37%) | 59 (34%) | 50 (32%) |

TABLE 7-2
Baseline to Week 4 all HRQOL and PRO Measures¹

| | | Week 4 − Wk 0 |
|---|---|---|
| | Mean (SD) | Δ^{2,3} |
| SF-36, Physical Component | | |
| Summary^{†} | | |
| Baseline | 32.8 (8.4) | 7.8* |
| Week 4 | 40.6 (9.3) | |
| SF-36, Mental Component | | |
| Summary^{†} | | |
| Baseline | 32.8 (13.0) | 10.1* |
| Week 4 | 42.9 (11.9) | |
| IBDQ, Total Score^{†} | | |
| Baseline | 124.6 (28.7) | 44.3* |
| Week 4 | 168.9 (27.8) | |
| FACIT-Fatigue Scale^{†} | | |
| Baseline | 22.9 (10.1) | 11.9* |
| Week 4 | 34.8 (11.0) | |
| Zung Depression Scale^{‡} | | |
| Baseline | 55.5 (11.4) | −9.6* |
| Week 4 | 46.0 (11.3) | |
| Abdominal Pain^{‡} | | |
| Baseline | 57.5 (21.0) | −28.6* |
| Week 4 | 29.0 (19.2) | |

¹Among 499 randomized responders at Week 4. Last observation carried forward.
²Δ represents the mean difference between week 4 and baseline scores.
³P-values performed using paired t-test.
*P < 0.0001.
^{†}Greater scores indicate greater quality of life.
^{‡}Greater scores indicate poorer quality of life.

TABLE 7-3
Zung Depression Scale Scores Through Week 56¹

| | Adalimumab | Adalimumab | | Adalimumab | |
|---|---|---|---|---|---|
| | Induction- | Maintenance Therapy | | Maintenance Therapy | |
| | Only | (40 mg eow) | | (40 mg Weekly) | |
| | (N = 168) | (N = 169) | | (N = 155) | |
| | Mean (SD) | Mean (SD) | Δ² | Mean (SD) | Δ² |
| Baseline | 55.2 (11.7) | 54.8 (11.4) | −0.42 | 56.7 (11.1) | 1.44 |
| Week 4 | 46.1 (11.9) | 44.9 (10.7) | −1.20 | 47.0 (11.2) | 0.96 |
| Week 12 | 47.4 (12.8) | 43.4 (11.0) | −4.05^{‡} | 46.1 (11.5) | −1.32 |
| Week 26 | 47.4 (12.7) | 43.7 (10.9) | −3.70^{†} | 46.3 (12.1) | −1.12 |
| Week 56 | 47.9 (13.1) | 43.7 (11.0) | −4.25^{‡} | 45.9 (12.3) | −2.00* |

¹Among 499 randomized responders at week 4. Last observation carried forward.
²Δ represents the difference between adalimumab eow or adalimumab weekly groups' mean scores vs. the mean score of the “Induction-Only” group.
*P < 0.05.
^{†}P < 0.01.
^{‡}P < 0.001.

TABLE 7-4
FACIT-Fatigue Scale Scores Through Week 56¹

| | | Adalimumab | | Adalimumab | |
|---|---|---|---|---|---|
| | Adalimumab | Maintenance | | Maintenenace | |
| | Induction- | Therapy | | Threapy | |
| | Only | (40 mg eow) | | (40 mg weekly | |
| | (N = 168) | (N = 169) | | (N = 155) | |
| | Mean (SD) | Mean (SD) | Δ² | Mean (SD) | Δ² |
| Baseline | 23.0 (10.2) | 23.5 (10.3) | 0.45 | 22.1 (9.9) | −0.92 |
| Week 4 | 34.6 (11.3) | 35.6 (10.6) | 1.01 | 34.2 (11.2) | −0.39 |
| Week 12 | 33.4 (12.2) | 38.2 (10.5) | 4.80^{‡} | 34.6 (11.5) | 1.14 |
| Week 26 | 32.8 (12.1) | 37.1 (11.2) | 4.22^{‡} | 34.4 (12.1) | 1.55 |
| Week 56 | 32.5 (12.6) | 36.8 (11.2) | 4.29^{‡} | 35.0 (12.7) | 2.48* |

¹Among 499 randomized responders at week 4. Last observation carried forward.
²Δ represents the difference between adalimumab eow or adalimumab weekly groups' mean scores vs. the mean score of the induction-only group.
*P < 0.05.
^{‡}P < 0.001.

TABLE 7-5
IBDQ Subscores Through Week 56¹

| | | Adalimumab | | Adalimumab | |
|---|---|---|---|---|---|
| | Adalimumab | Maintenance | | Maintenance | |
| | Induction- | Therapy | | Therapy | |
| | Only | (40 mg eow) | | (40 mg weekly | |
| | (N = 140) | (N = 129) | | (N = 114) | |
| | Mean (SD) | Mean (SD) | Δ² | Mean (SD) | Δ² |
| Social | | | | | |
| Function | | | | | |
| Baseline | 21.9 (7.5) | 21.7 (7.4) | −0.2 | 21.6 (7.5) | −0.3 |
| Week 4 | 28.3 (6.7) | 29.2 (5.5) | 0.9 | 28.7 (6.0) | 0.4 |
| Week 12 | 27.7 (7.3) | 30.1 (5.2) | 2.4^{‡} | 29.7 (6.1) | 2.0^{†} |
| Week 26 | 27.3 (7.3) | 30.4 (5.3) | 3.1^{‡} | 29.6 (6.2) | 2.3^{†} |
| Week 56 | 27.4 (7.3) | 30.7 (5.1) | 3.3^{‡} | 30.0 (6.3) | 2.6^{‡} |
| Systemic | | | | | |
| Function | | | | | |
| Baseline | 15.8 (5.2) | 16.4 (4.9) | 0.6 | 15.7 (4.9) | −0.1 |
| Week 4 | 23.4 (5.7) | 24.1 (5.1) | 0.7 | 23.3 (50) | 0.0 |
| Week 12 | 22.3 (6.5) | 24.8 (5.8) | 2.6^{†} | 23.6 (5.6) | 1.3 |
| Week 26 | 21.9 (6.7) | 24.8 (6.1) | 2.9^{‡} | 23.4 (5.9) | 1.5* |
| Week 56 | 21.9 (6.8) | 24.7 (5.8) | 2.8^{‡} | 23.6 (6.3) | 1.7* |
| Emotional | | | | | |
| Function | | | | | |
| Baseline | 48.0 (13.6) | 48.9 (12.7) | 0.9 | 46.5 (12.3) | −1.5 |
| Week 4 | 63.8 (13.0) | 63.8 (12.1) | 0.0 | 61.7 (12.0) | −2.1 |
| Week 12 | 60.7 (14.2) | 65.9 (12.8) | 5.2^{†} | 63.9 (13.4) | 3.2^{†} |
| Week 26 | 59.9 (14.8) | 65.9 (12.9) | 6.1^{‡} | 63.2 (13.4) | 3.4^{†} |
| Week 56 | 59.9 (14.6) | 66.7 (12.7) | 6.8^{‡} | 64.0 (4.3) | 4.0^{†} |
| Bowel | | | | | |
| Function | | | | | |
| Baseline | 39.2 (8.7) | 39.5 (8.1) | 0.2 | 38.5 (8.7) | −0.7 |
| Week 4 | 52.8 (9.5) | 53.7 (8.0) | 1.0 | 53.3 (8.1) | 0.5 |
| Week 12 | 50.2 (9.4) | 54.6 (9.0) | 4.4^{‡} | 54.4 (9.7) | 4.2^{‡} |
| Week 26 | 50.1 (9.9) | 55.1 (9.0) | 5.0^{‡} | 54.9 (10.0) | 4.8^{‡} |
| Week 56 | 50.3 (10.2) | 55.1 (9.0) | 4.8^{‡} | 55.6 (10.2) | 5.3^{‡} |

¹Among 499 randomized responders at Week 4. Last observation carried forward.
²Δ represents the difference between adalimumab eow or adalimumab weekly groups' mean scores vs. the mean score of the induction-only group.
*P < 0.05.
^{†}P < 0.01.
^{‡}P < 0.001.

TABLE 7-6
Normalized and Standardized SF-36 Scores Through Week 56¹

| | Adalimumab | | Adalimumab | |
|---|---|---|---|---|
| | Maintenance | | Maintenance | |
| Adalimumab | Therapy | | Therapy | |
| Induction-Only | (40 mg eow) | | (40 mg weekly) | |
| (N = 106) | (N = 140) | | (N = 128) | |
| Mean (SD) | Mean (SD) | Δ² | Mean (SD) | Δ² |

Physical Component Summary

| | | | | | |
|---|---|---|---|---|---|
| Baseline | 32.7 (8.9) | 32.8 (8.8) | 0.13 | 32.8 (7.6) | 0.15 |
| Week 4 | 40.7 (9.9) | 41.0 (8.7) | 0.29 | 40.0 (9.4) | −0.70 |
| Week 12 | 41.0 (10.0) | 43.7 (9.5) | 2.74^{†} | 42.6 (9.6) | 1.64 |
| Week 26 | 41.1 (9.5) | 44.2 (10.1) | 3.07^{†} | 42.8 (9.6) | 1.67 |
| Week 56 | 41.8 (9.4) | 44.2 (9.3) | 2.43* | 43.9 (10.3) | 2.06 |

Mental Component Summary

| | | | | | |
|---|---|---|---|---|---|
| Baseline | 33.7 (13.2) | 33.6 (13.1) | −0.11 | 31.2 (12.7) | −2.52 |
| Week 4 | 44.1 (12.3) | 42.6 (12.3) | −1.49 | 42.2 (11.2) | −1.87 |
| Week 12 | 42.8 (13.1) | 45.1 (12.6) | 2.28 | 42.7 (14.0) | −0.13 |
| Week 26 | 42.3 (13.5) | 44.8 (12.5) | 2.56 | 42.5 (13.8) | 0.27 |
| Week 56 | 42.2 (13.3) | 45.5 (12.3) | 3.30* | 43.0 (14.4) | 0.75 |

¹Among 499 randomized responders at Week 4. Last observation carried forward.
²Δ represents the difference between adalimumab eow or adalimumab weekly groups' mean scores vs. the mean score of the induction-only group.
*P < 0.05.
^{†}P < 0.01.

TABLE 7-7
Abdominal Pain VAS Score Through Week 56¹

| | | Adalimumab | | Adalimumab | |
|---|---|---|---|---|---|
| | Adalimumab | Maintenance | | Maintenance | |
| | Induction- | Therapy | | Therapy | |
| | Only | (40 mg eow) | | (40 mg weekly | |
| | (N = 167) | (N = 169) | | (N = 155) | |
| | Mean (SD) | Mean (SD) | Δ² | Mean (SD) | Δ² |
| Baseline | 57.6 (22.2) | 56.7 (18.6) | −0.86 | 58.3 (22.1) | 0.69 |
| Week 4 | 28.3 (19.5) | 27.8 (19.4) | −0.49 | 31.0 (18.7) | 2.67 |
| Week 12 | 32.9 (24.5) | 24.0 (21.2) | −8.92^{‡} | 27.8 (23.1) | −5.11* |
| Week 26 | 34.8 (25.5) | 22.5 (21.1) | −12.28^{‡} | 26.8 (23.7) | −7.93^{†} |
| Week 56 | 36.0 (26.5) | 23.9 (22.4) | −12.10^{‡} | 26.7 (24.2) | −9.35^{‡} |

¹Among 499 randomized responders at Week 4. Last observation carried forward.
²Δ represents the difference between adalimumab eow or adalimumab weekly groups' mean scores vs. the mean score of the induction-only group.
*P < 0.05.
^{†}P < 0.01.
^{‡}P < 0.001.

### REFERENCES

1. Munkholm P, Langholz E, Davidsen M, et al. Disease activity courses in a regional cohort of Crohn's disease patients. Scand J Gastroenterol 1995; 30:699-706.
2. Cohen R D. The quality of life in patients with Crohn's disease. Aliment Pharmacol Ther 2002; 9:1603-9.
3. Wilson A, Reyes E, Ofman J. Prevalence and outcomes of anemia in inflammatory bowel disease: a systematic review of the literature. Am J Med 2004; 116(Suppl 7A):44S-49S.
4. Longobardi T, Jacobs P, Bernstein C N. Work losses related to inflammatory bowel disease in the United States: Results from the National Health Interview Survey. Am J Gastroenterol 2003; 98 (5):1064-1072.
5. Fuller-Thomson E, Sulman J. Depression and inflammatory bowel disease: Findings from two nationally representative Canadian surveys. Inflamm Bowel Dis 2006; 12:697-707.
6. Guyatt G, Mitchell A, Irvine E J, et al. A new measure of health status for clinical trials in inflammatory bowel disease. Gastroenterology 1989; 96:804-10.
7. Ware J E, Sherbourne C D. The MOS 36-item short-form health survey (SF-36). I. Conceptual framework and item selection. Med Care 1992; 30:473-83.
8. Feagan B G, Yan S, Bala, M, et al. The effects of infliximab maintenance therapy on health-related quality of life. Am J Gastroenterol 2003; 98:2232-8.
9. Colombel J F, Sandborn W J, Rutgeerts P, et al. Adalimumab for maintenance of clinical response and remission in patients with Crohn's disease: The CHARM trial. Gastroenterology 2007; 132:52-65.
10. Biggs J T, Wylie L T, Ziegler V E. Validity of the Zung self-rating depression scale. Br J Psychiatry 1978; 132:381-5.
11. McDowell I, Newell C. Measuring health: A guide to rating scales and questionnaires, 2^{nd} ed. New York: Oxford University Press, 1996.
12. Cella D, Lai J S, Chang C H, et al. Fatigue in cancer patients compared with fatigue in the general United States population. Cancer 2002; 94:2528-38.
13. Webster K, Cella D, Yost K. The functional assessment of chronic illness therapy (FACIT) measurement system: Properties, applications, and interpretation. Health Qual Life Outcomes 2003; 1:79.
14. Irvine E J, Feagan B, Rochon J, et al. Quality of life: A valid and reliable measure of therapeutic efficacy in the treatment of inflammatory bowel disease. Canadian Crohn's Relapse Prevention Trial Study Group. Gastroenterology 1994; 106:287-96.
15. Pallis A G, Mouzas L A, Vlachonikolis I G. The inflammatory bowel disease questionnaire: A review of its national validation studies. Inflamm Bowel Dis 2004; 10:261-9.
16. Irvine E J. Development and subsequent refinement of the inflammatory bowel disease questionnaire: A quality-of-life instrument for adult patients with inflammatory bowel disease. J Pediatr Gastroenterol Nutr 1999; 28:S23-S27.
17. Gregor J C, McDonald J W D, Klar N, et al. An evaluation of utility measurement in Crohn's disease. Inflamm Bowel Dis 1997; 3:265-76.
18. Brazier J E, Harper R, Jones N M, et al. Validating the SF-36 health survey questionnaire: new outcome measure for primary care. BMJ 1992; 305:160-4.
19. Ware J E Jr, Gandek B, Kosinski M, Aaronson N K, Apolone G, Brazier J, Bullinger M, Kaasa S, Leplege A, Prieto L, Sullivan M, Thunedborg K. The equivalence of SF-36 summary health scores estimated using standard and country-specific algorithms in 10 countries: results from the IQOLA Project. International Quality of Life Assessment. J Clin Epidemiol 1998; 51(11)167-1170.
20. Kosinski, M, Zhao S Z, Dedhiya S, et al. Determining minimally important changes in generic and disease-specific health related quality of life questionnaires in clinical trials of rheumatoid arthritis. Arthritis Rheum 2000; 43:1478-87.
21. Surtees P G, Wainwright N W, Khaw K T, Day N E. Functional health status, chronic medical conditions and disorders of mood. Br J Psychiatry 2003 October; 183:299-303
22. Patrick D L, Gagnon D D, Zagari M J, et al. Epoetin Alfa Study Group. Assessing the clinical significance of health-related quality of life (HRQOL) improvements in anaemic cancer patients receiving epoetin alfa. Eur J Cancer 2003; 39:335-45.

### Example 8

### Sustainability of Adalimumab in Improving the Quality of Life of Patients With Fistulizing Crohn's Disease: 2-Year Data From CHARM

Fistulas occur in 17%-43% of patients with Crohn's disease (CD) [1]. A previous analysis has demonstrated that adalimumab (ADA) improved qualify of life (QOL) among patients with fistulizing CD [2]. The following example examines the long-term efficacy of ADA on QOL in patients with fistulizing disease through 2 years after enrollment in the CHARM trial.

Patients in CHARM were randomized to placebo, ADA 40 mg every other week (EOW) or 40 mg weekly (EW). At or after Week 12, patients with flare (increase in CDAI of ≧70 points compared with Week 4 and CDAI>220) or non-response (did not attain a CDAI decrease of ≧70 points compared with baseline) were switched to open-label (OL) ADA 40 mg EOW. At the end of CHARM (56 weeks), patients could enroll into an open-label extension (OLE) where those who completed CHARM on blinded therapy received OL EOW and those already on OL maintained their therapy. In the OLE, patients could change from EOW to EW for flares or non-response. Pooling the 2 dosage groups, the analyses included patients with fistulas at baseline of CHARM assigned to ADA EOW or EW. QOL measures evaluated included IBDQ, and SF-36 Physical (PCS) and Mental (MCS) component summaries over time. Data were described and compared with baseline using the paired t-test. Last observation carried forward (LOCF) analyses were used. The proportion of patients with IBDQ>170 (which correlates with clinical remission) was calculated using both non-responder imputation (NRI) and LOCF.

Table 8-1 presents mean QOL measures for all fistulizing patients (N=48), EOW and EW treatment groups combined. Using LOCF, at Week 56 and 116, the percentage of patients achieving IBDQ>170 were 54.2% and 60.4% in the combined group. The non-responder imputation yielded similar results.

TABLE 8-1
Long-Term Efficacy: QOL Measures Among Fistulizing Pts (N = 48)

| | IBDQ | SF-36 PCS | SF-36 MCS |
|---|---|---|---|
| Time | Mean (SD) | Mean (SD) | Mean (SD) |
| CHARM | 124 (29) | 36 (8) | 38 (12) |
| baseline | | | |
| Week 56 | 169 (36) | 47 (9) | 46 (12) |
| Week 92 | 175 (33) | 47 (9) | 47 (12) |
| Week 116 | 171 (37) | 46 (10) | 47 (12) |

Combined EOW and EW therapy.
All p < 0.05 vs. baseline, LOCF.

In conclusion, the majority of fistulizing patients treated with ADA show significant QOL improvements over a 2-year period, demonstrating the sustainable efficacy of ADA in this hard-to-treat subset of CD patients.
[1] Schwartz, D A, et al. Aliment Pharmacol Ther. 2004; 19(9):953-967.
[2] Colombel J F, et al. Am J Gastroenterol. 2007; 102(Suppl 2) 5457.

### Example 9

### Adalimumab Sustains Quality-of-Life Improvements in Patients with Crohn's Disease: 2-Year Data From CHARM

The objective of the following study was to assess long-term effects of ADA on QOL in patients with CD through 2 years (yrs) from CHARM baseline (BL).

In CHARM, pts were randomized to placebo, 40 mg ADA every other wk (EOW), or 40 mg ADA wkly (EW). Patients with flare/non-response could receive open-label (OL) ADA at/after Wk 12. At the end of CHARM (56 wks), patients could enroll in an OL extension (OLE) in which those on blinded therapy received ADA EOW and those already on OL ADA maintained their therapies. In CHARM and OLE, patients could change from EOW to EW dosage for flares/non-response. In this analysis, patients initially randomized to ADA in CHARM were followed through 2 yrs of exposure. The percentage of patients from each originally randomized ADA group with IBDQ>170 (which correlates with clinical remission) was calculated using both last observation carried forward (LOCF) and non-responder imputation (NRI). LOCF analyses were performed for total IBDQ values and SF-36 Mental Component Summary (MCS) and Physical Component Summary (PCS) scores over time for EOW, EW, and combined ADA groups. Paired t-tests compared values at each visit with BL values.

Of 328 patients who entered the OLE, 144 had been randomized to ADA EOW to and 184 had been randomized to ADA EW in CHARM. The percentages (LOCF) of pts achieving IBDQ>170 at Wks 56 and 116 were 63.2% and 54.9% in EOW, 59.8% and 59.2% in EW, and 61.3% and 57.3% in the combined EOW+EW groups, respectively. NRI yielded similar results. Mean total IBDQ and SF-36 PCS and MCS scores through 2 yrs are presented (Table 9-1). Overall, patients sustained QOL improvements over 2 yrs with ADA maintenance.

TABLE 9-1
QOL Improvements With ADA Over 2 Yrs

| QOL | | | |
|---|---|---|---|
| ADA Group | CHARM BL | Wk 56 | Wk 116 |
| in CHARM | Mean (SD) | Mean (SD) | Mean (SD) |
| Total IBDQ | | | |
| EOW, n = 144 | 125 (30) | 178* (30) | 170* (36) |
| EW, n = 184 | 123 (28) | 173* (30) | 169* (35) |
| EOW + EW, | 124 (29) | 175* (31) | 170* (36) |
| n = 328 | | | |
| SF-36 PCS, MCS | | | |
| EOW, n = 144 | 37 (8), 38 (11) | 48* (8), 49* | 46* (11), 47* (11) |
| | | (10) | |
| EW, n = 184 | 36 (6), 37 (11) | 47* (9), 48* | 46* (10), 46* (12) |
| | | (11) | |
| EOW + EW, | 36 (7), 38 (11) | 47* (9), 48* | 46* (10), 46* (11) |
| n = 328 | | (11) | |

*P < 0.05 vs. BL. LOCF.

In conclusion, clinically important improvements in QOL achieved with ADA in the CHARM trial were sustained through 2 yrs of ADA maintenance therapy.
[1] Colombel J-F, et al. Gastroenterology. 2007; 132:52-65.
[2] Loftus E V, et al. Am J Gastroenterol. 2007; 102(52):S457.

### Example 10

### Adalimumab Maintains Long-Term Remission in Moderately to Severely Active Crohn's Disease Through 2 Years

The ability of adalimumab treatment to maintain long-term remission in moderately to severely active CD through a 2 year timespan was examined in 328 patients from the CHARM trial.

Patients received treatment as described above in Example 7. Patients randomized to placebo in CHARM were not analyzed. Various OLE outcome measurements were utilized: remission (CDAI<150), evaluated by the maintenance of remission at Months 18 and 24 from baseline of the CHARM study (i.e., Months 6 and 12 of the OLE); maintenance of response (clinical response 70 and 100 (CR-70 and CR-100), defined as a decrease from baseline CDAI of greater than or equal to 70 points or 100 points, respectively); change in IBDQ score; and improvement in SF-36 PCS and MCS scores over time. Baseline demographics are provided below in Table 10-1.

TABLE 10-1
Baseline Demographics

| | All ADA |
|---|---|
| Characteristic | (n = 328) |
| Male, % | 36 |
| Age (yrs), mean | 38 |
| Body weight (kg), mean | 72 |
| C-reactive protein (mg/dL), mean | 2.0 |
| Baseline CDAI score, mean | 308 |
| Previous corticosteroid exposure, % | 41 |
| Previous TNF-antagonist exposure, % | 46 |

*Patients randomized to adalimumab 40 mg every other week (EOW) or 40 mg every week (EW) who rolled over into OLE.
ADA, adalimumab.

Tables 10-2 and 10-3 below gives the results for long-term maintenance of remission and maintenance of CR-70 for 18 and 24 month post-56 weeks from the study double-blind period. Patients were randomized to adalimumab and in remission at Week 56 of CHARM, and subsequently enrolled in OLE.

TABLE 10-2
Patients in remission at end of period (months post-Week 56)

| Patient | | |
|---|---|---|
| Population | 18 months | 24 months |
| NRI/ITT | 78% | 77% |
| LOCF/ITT | 81% | 85% |
| NRI/RR | 81% | 79% |
| LOCF/RR | 84% | 87% |

ITT, intention-to-treat;
LOCF, last observation carried forward;
NRI, nonresponder imputation;
RR, randomized responder.
ITT (n = 145) and RR (n = 123).

TABLE 10-3
Patients maintaining CR-70 at end of period (months post-Week 56)

| Patient | | |
|---|---|---|
| Population | 18 months | 24 months |
| NRI/ITT | 88% | 83% |
| LOCF/ITT | 92% | 92% |
| NRI/RR | 89% | 83% |
| LOCF/RR | 92% | 91% |

ITT, intention-to-treat;
LOCF, last observation carried forward;
NRI, nonresponder imputation;
RR, randomized responder.
ITT (n = 145) and RR (n = 123).

Tables 10-4 and 10-5 below gives the results for long-term maintenance of remission and maintenance of CR-70 for 18 and 24 month post-26 weeks from the study double-blind period. Patients were randomized to adalimumab and in remission at Week 26 of CHARM, and subsequently enrolled in OLE.

TABLE 10-4
Patients in remission at end of period (months post-Week 26)

| Patient | | |
|---|---|---|
| Population | 18 months | 24 months |
| NRI/ITT | 66% | 69% |
| LOCF/ITT | 71% | 76% |
| NRI/RR | 70% | 73% |
| LOCF/RR | 74% | 79% |

ITT, intention-to-treat;
LOCF, last observation carried forward;
NRI, nonresponder imputation;
RR, randomized responder.
ITT (n = 148) and RR (n = 126).

TABLE 10-5
Patients maintaining CR-70 at end of period (months post-Week 26)

| Patient | | |
|---|---|---|
| Population | 18 months | 24 months |
| NRI/ITT | 77% | 74% |
| LOCF/ITT | 82% | 81% |
| NRI/RR | 80% | 76% |
| LOCF/RR | 85% | 84% |

ITT, intention-to-treat;
LOCF, last observation carried forward;
NRI, nonresponder imputation;
RR, randomized responder.
ITT (n = 194) and RR (n = 157).

Mean IBDQ scores rose from between 120 and 130 at CHARM baseline to over 160 at Week 4; at one year post-CHARM baseline, mean IBDQ scores were over 170 and remained at or above 170 at the assessments at around 24 months and at 29 months. Patients maintained the improvement seen in IBDQ scores after Week 56, as shown to Table 10-6.

TABLE 10-6
Patients Receiving ADA in CHARM and OLE Who Achieved
IBDQ = 170

| Time of Evaluation | % Achievers |
|---|---|
| CHARM Baseline | 5 |
| Week 4 | 41 |
| 1 Year | 61 |
| ~24 Months | 60 |
| 29 Months | 57 |

LOCF. McNemar test. IBDQ ≧ 170 correlates with clinical remission.

Tables 10-7 and 10-8 below gives the results for long-term maintenance of CR-100 for 18 and 24 month post-56 and post-26 weeks, respectively, from the study double-blind period. Patients were randomized to adalimumab and in remission at Week 56 or Week 26 of CHARM, and subsequently enrolled in OLE.

TABLE 10-7
Patients maintaining CR-100 at end of period (months post-Week 52)

| Patient | | |
|---|---|---|
| Population | 18 months | 24 months |
| NRI/ITT | 85% | 81% |
| LOCF/ITT | 88% | 89% |
| NRI/RR | 87% | 81% |
| LOCF/RR | 90% | 89% |

ITT, intention-to-treat;
LOCF, last observation carried forward;
NRI, nonresponder imputation;
RR, randomized responder.
ITT (n = 182) and RR (n = 149).

TABLE 10-8
Patients maintaining CR-100 at end of period (months post-Week 26)

| Patient | | |
|---|---|---|
| Population | 18 months | 24 months |
| NRI/ITT | 76% | 73% |
| LOCF/ITT | 80% | 79% |
| NRI/RR | 81% | 77% |
| LOCF/RR | 84% | 83% |

ITT, intention-to-treat;
LOCF, last observation carried forward;
NRI, nonresponder imputation;
RR, randomized responder.
ITT (n = 182) and RR (n = 149).

Improvement in Short Form 36 (SF-36) scores with adalimumab treating over time is shown in Tables 10-9 (PCS) and 10-10 (MCS).

TABLE 10-9
Improvement in SF-36 PCS with Adalimumab Over Time

| | Mean SF-36 |
|---|---|
| Time of Evaluation | PCS Scores |
| CHARM Baseline | 36 |
| Week 4 | 43 |
| 1 Year | 47 |
| ~24 Months | 46 |
| 29 Months | 46 |

LOCF.

TABLE 10-10
Improvement in SF-36 MCS with Adalimumab Over Time

| | Mean SF-36 |
|---|---|
| Time of Evaluation | MCS Scores |
| CHARM Baseline | 38 |
| Week 4 | 45 |
| 1 Year | 48 |
| ~24 Months | 47 |
| 29 Months | 47 |

LOCF

Adverse event data is presented in Table 10-11 below.

TABLE 10-11
Adverse Events of Interest

| Adverse events (AE), n = 315 | E (E/100-PYs) |
|---|---|
| Any AE | 802 (93.9) |
| Any serious AE | 213 (24.9) |
| Any AE leading to discontinuation | 170 (19.9) |
| Infectious AE | 507 (59.4) |
| Serious infections | 53 (6.2) |
| Malignant neoplasms | 12 (1.4) |
| Injection-site pain AE | 54 (6.3) |
| Opportunistic infections | 19 (2.2) |
| Congestive heart failure | 0 |
| Demyelinating disease | 2 (0.2) |
| Any fatal AE | 2 (0.2) |

In conclusion, Adalimumab showed sustained efficacy in maintaining CD resmission through 2 years of therapy. The vast majority of ADA-treated patient in remission after 1 year in CHARM maintained remission for an additional year in an OLE. Patients randomized to ADA in CHARM achieved clinically important improvements in quality of life, as measured by th IBDQ, and the QOL improvements were maintained over 2 years of ADA maintenance therapy.

### Example 11

### Adalimumab Maintains Long-Term Remission in Moderately to Severely Active Crohn's Disease After Infliximab Failure: 1-Year Follow-Up

In the study described below, the ability of adalimumab to induce remission in Crohn's Disease patients who were either intolerant of or lost responsiveness to infliximab was studied. After an initial screening period, 325 patients were placed in a double-blind, placebo controlled study for four weeks (160 mg at Wk 0 and 80 mg at Wk 2, or PBO). At week 4 (primary endpoint), patients entered a long-term, open-label extension (OLE) study (Sandborn W J, et al. Ann Intern Med 2007, 146(12): 829-838).

Table 11-1 shows efficacy outcomes at Week 4 for remission and patients achieving a CR-70 and CR-100 response, in percentages and numbers of patients (achievers/total).

TABLE 11-1
Efficacy Outcomes

| | Placebo | 160/80 mg |
|---|---|---|
| Remission* | 7% (12/166) | 21% (34/159) |
| CR-70 Response* | 34% (56/166) | 52% (82/159) |
| CR-100 Response† | 25% (41/166) | 38% (61/159) |

*p < 0.001,
†p < 0.01, both vs placebo.
CR-70, decrease in Crohn's Disease Activity Index (CDAI) ≧70 points;
CR-100, decrease in CDAI ≧100 points;
remission, CDAI <150 points.
Sandborn WJ, et al. Ann Intern Med 2007; 146(12): 829-38.

In the OLE period of the study, patients were administered 40 mg adalimumab EOW, with the option to adjust to 40 mg EW in case of flare/non-responsiveness. Patients were evaluated at 6 month and 12 months.

Table 11-2 shows the long-term maintenance of remission data, for intent to treat (ITT; placebo and adalimumab patients included) and randomized responder (RR, patients at week 4 who attained a CR-70 response and enrolled in OLE) patient groups. The table provides data for the 6-month OLE to 12-month OLE period (patients who were in remission at 6 months in OLE and still in remission at 1 year OLE).

TABLE 11-2
Long Term Remission

| | ITT | RR |
|---|---|---|
| 6-month OLE | 35% (107/310) | 57% (72/126) |
| 12-month OLE | 29% (89/310) | 40% (50/126) |
| 6 mo. OLE to 12 mo. OLE | 62% (66/107) | 57% (33/58) |

Tables 11-3 and 11-4 present data for the long-term maintenance of a CR-100 response and a CR-70 response, respectively.

TABLE 11-3
Maintenance of a CR-100 Response

| | ITT | RR |
|---|---|---|
| 6-month OLE | 51% (157/310) | 75% (94/126) |
| 12-month OLE | 44% (135/310) | 66% (83/126) |

ITT includes placebo and adalimumab patients.

TABLE 11-4
Maintenance of a CR-70 Response

| | ITT | RR |
|---|---|---|
| 6-month OLE | 60% (187/310) | 92% (116/126) |
| 12-month OLE | 50% (156/310) | 65% (82/126) |

ITT includes placebo and adalimumab patients.

Table 11-5 presents the adverse events of interest.

TABLE 11-15
Adverse Events

| Adverse events (AE), n = 315 | E (E/100-PYs) | |
|---|---|---|
| Any AE | 2,625 | (960.1) |
| Any serious AE | 117 | (42.8) |
| Any AE leading to discontinuation | 77 | (28.2) |
| Infectious AE | 431 | (160.6) |
| Serious infections | 19 | (6.9) |
| Malignant neoplasms | 8 | (2.9) |
| Injection-site pain AE | 11 | (4.0) |
| Opportunistic infections | 6 | (2.2) |

| | |
|---|---|
| Congestive heart failure | 0 |
| Demyelinating disease | 0 |
| Any fatal AE | 0 |

E/100-PYs, events per 100-patient-years.

In conclusion, adalimumab demonstrated sustained efficacy in maintaining clincial remission and response through 1 year of therapy for CD in patients who failed prior infliximab therapy. Two-thirds of responding patients maintained response and 40% achieved long-term remission. Safety was consistent with over 10 years of clinical experience with adalimumab.

### Example 12

### Health-Related Quality of Life (HRQOL) and Work Productivity Outcomes for Psoriasis Patients in Europe

### Background:

Psoriasis (Ps) dramatically affects patients' HRQOL and daily to functioning. Ps often leads to work impairment in 2 forms: absenteeism and presenteeism (reduced productivity while at work).

### Objectives:

With data on patient-reported outcomes, this study investigated the extent to which Ps impairs patients' HRQOL, or leads to absence from work, reduced productivity while at work, and decreases in daily activities.

### Methods:

The study consisted of respondents to a 2007 EU National Health and Wellness Survey (NHWS), an annual cross-sectional survey of representative samples of adults (≧18 years) from 5 countries (France, Germany, Italy, Spain, UK). The Internet survey was completed by 53,524 respondents in June 2007. We assumed self-reports of a diagnosis of Ps were bona fide. All respondents completed a self-administered questionnaire, which included the Short Form 12 (SF-12) and Work Productivity and Activity Impairment (WPAI) questionnaire. Results were stratified by disease severity based on BSA affected (mild, <2% BSA; moderate, 2-10%; severe, 10%) and compared with the non-Ps population. Bivariate analyses compared subgroups. Statistical testing employed Z-test in Quantum for percentages and t-tests for means.

### Results:

2,288 respondents reported being diagnosed with Ps by a physician. Of these, 598 reported moderate (n=484) or severe (n=114) disease. SF-12 results demonstrated poor quality of life, via both the Physical and Mental Component Summary scores, especially for patients with moderate and severe Ps. Overall, moderate and severe Ps patients reported significantly lower mental and physical health status than mild and non-Ps sufferers (PCS: 48.51, 46.62, 45.18, and 43.09; MCS: 46.86, 45.43, 42.74, and 41.12, for non-Ps, mild, moderate and severe Ps, respectively, p<0.05). For employed patients with severe Ps, a mean 4-hour absence from work per week was reported, vs. 2 hours per week for mild and non-Ps sufferers (p<0.05). For patients with moderate Ps, a mean of 3 hours' absence from work per week was reported, which was greater than the absence of non-Ps sufferers (p<0.05). If those with absenteeism alone (19%) were considered, mean hours missed were 16/week and 18/week for moderate and severe Ps patients, respectively. Moderate and severe Ps sufferers had greater work productivity and activity impairment vs. mild or non-Ps sufferers (p<0.05). Severe Ps sufferers had 29% impairment in work productivity and 28% impairment in daily activities, while moderate Ps patients had 24% and 25% impairments respectively. By comparison, mild Ps patients/non-Ps sufferer reported lower reductions in work impairment (20%/17%) and daily activities (19%/17%).

### Conclusions:

Psoriasis has both physical and mental impacts, which increase with severity. Associated loss in work productivity and daily activities has considerable costs to society. Therapies that help patients achieve mild disease or remission improve HRQOL and work productivity outcomes.

### Example 13

### Effect of Adalimumab Treatment on C-Reactive Protein in Patients With Moderate to Severe Psoriasis

### Background:

C-reactive protein (CRP), a biomarker of systemic inflammation, is elevated in patients with psoriasis (Ps) and is a predictor of future cardiovascular risk. Recent studies have demonstrated that patients with moderate to severe Ps are at increased risk for cardiovascular disease.

### Objectives:

To evaluate CRP concentrations in patients with moderate to severe Ps before and after treatment with adalimumab.

### Methods:

REVEAL (Randomized Controlled Evaluation of Adalimumab Every Other Week Dosing in Moderate to Severe Psoriasis Trial) was a 52-week (wk), double-blind, randomized, placebo-controlled, Phase III trial of adalimumab in patients with moderate to severe chronic plaque Ps conducted in the United States and Canada. During the first 16 weeks (Period A), patients were treated with adalimumab (80 mg at Wk 0, 40 mg every other wk from Wk 1-15) or placebo. This post-hoc analysis of REVEAL evaluated CRP concentrations in Ps patients at baseline and after 16 wks of treatment with adalimumab or placebo. Changes in CRP concentrations were also evaluated in subgroups with or without self-reported psoriatic arthritis (PsA) and with or without obesity (by body mass index [BMI]). The CRP assay had an upper limit of normal of 9.0 mg/L. The lower limit of detection was 4.0 mg/L; results 4.0 were recorded as 4.0 mg/L.

### Results:

Of 1,212 patients in REVEAL, 814 were randomized to adalimumab and 398 were randomized to placebo. At baseline, 28% of patients had PsA, and the mean BMI was 31 kg/m²(obese). For patients in the Ps group (i.e., without self-reported PsA), the mean CRP concentrations at baseline were 6.5 mg/L (range 4.0-70.8) for adalimumab-treated patients and 6.4 mg/L (range 4.0-61.3) for placebo-treated patients; the mean changes from baseline in CRP were −1.3 vs. 0.3 (p<0.01). Of those patients in the Ps group with an elevated CRP at baseline, 64.6% of adalimumab patients (53 of 82) vs. 33.3% of PBO patients (13 of 39) had a normal CRP at Wk 16 (p=0.0017). For patients in the PsA group, the mean CRP concentrations at baseline were 11.6 mg/L (range 4.0-206.0) for adalimumab-treated patients and 9.7 mg/L (range 4.0-106.0) for PBO-treated patients; the mean changes from baseline in CRP were −6.3 vs. −1.9 (p<0.01). Of those patients in the PsA group with an elevated CRP at baseline, 71.9% of adalimumab patients (41 of 57) vs. 42.9% of placebo patients (12 of 28) had a normal CRP at Week 16 (p=0.017). Of patients who were obese, 64.1% of adalimumab patients (59 of 92) and 36% of placebo patients (18 of 50) changed from an elevated CRP concentration at baseline to a normal CRP at Week 16 (p=0.0016).

### Conclusions:

Among patients with moderate to severe Ps, CRP concentrations were greater for those with PsA than with those with Ps alone. Adalimumab treatment led to significant reductions in CRP concentrations, regardless of the presence of PsA or obesity.

### Example 14

### Efficacy Outcomes for Patients with Psoriasis Who Interrupt Adalimumab Therapy

### Background:

Adalimumab is a fully human, IgG¹ monoclonal antibody specific for tumor necrosis factor (TNF), a potent inflammatory cytokine.

### Objective:

The purpose of this analysis was to determine whether interruption of adalimumab therapy affected efficacy outcomes in patients with psoriasis.

### Methods:

Patients with moderate to severe psoriasis (Psoriasis Area and Severity Index [PASI] score ≧12) who previously failed topical therapy and were anti-TNF therapy-naïve were enrolled in REVEAL (Randomized Controlled Evaluation of Adalimumab Every Other Week Dosing in Moderate to Severe Psoriasis Trial), a 52-week (wk) study in which patients received adalimumab 40 mg every other wk. At Wk 33 of REVEAL, patients who had initially been randomized to adalimumab and who had achieved a PASI 75 response vs. baseline were re-randomized in a double-blind manner to either continued adalimumab or placebo. Upon loss of adequate response (defined as <PASI 50 relative to baseline and at least a 6-point increase in PASI score relative to Wk 33), patients were eligible to receive adalimumab in an open-label extension (OLE). All patients who completed Wk 52 of REVEAL were eligible to receive adalimumab in the OLE. This analysis compared PASI 75 response rates between patients re-randomized at Wk 33 of REVEAL to placebo or adalimumab, for patients who lost or did not lose adequate response during REVEAL, using data from Wk 24 of the OLE relative to baseline.

### Results:

The percentage of patients who lost adequate response was significantly greater among those re-randomized to placebo (28%, 68/240) compared with those re-randomized to adalimumab (5%, 12/250) (p<0.001). Among patients re-randomized to the placebo group, PASI 75 response rates were 55% for patients who lost adequate response vs. 84% for patients who did not lose adequate response. Among patients re-randomized to adalimumab, PASI 75 response rates after 24 weeks of re-treatment in OLE were 55% for patients who lost adequate response vs. 83% for patients who did not lose adequate response.

### Conclusions:

Interruption of adalimumab was significantly associated with loss of adequate response. Patients who lost adequate response were less likely to achieve their previous efficacy upon re-treatment compared with patients who resumed treatment without prior loss of adequate response. These results suggest that adalimumab therapy should be used continuously in psoriasis treatment.

### Example 15

### Benefits of Loading Dose in an Adalimumab Therapeutic Regimen for Moderate to Severe Psoriasis

### Aims:

The primary goal of adalimumab treatment for moderate to severe chronic plaque psoriasis is to induce and maintain clinical response. In addition, improving patients' quality of life with an early clinical response may increase treatment compliance. This analysis employed Phase II clinical results and a modeling and simulation approach to evaluate time to achieving efficacious steady-state drug concentration with a single 80-mg loading dose of adalimumab.

### Methods:

In a Phase II, 12-week, placebo-controlled trial, patients with moderate to severe chronic plaque psoriasis were randomized to one of three arms: placebo (n=52); adalimumab 80 mg at Week 0, followed by 40 mg every other week (eow) beginning at Week 1 (n=45); or adalimumab 80 mg at Weeks 0 and 1, followed by 40 mg weekly beginning at Week 2 (n=50).¹ Adalimumab serum concentrations were measured by ELISA at baseline and Weeks 1, 2, 4, 8, 11, and 12.¹ A regimen of adalimumab 40 mg eow without a loading dose was not studied in this trial. Therefore, a population pharmacokinetic (PK) model was developed using the concentration data from the Phase II trial and clinical trial simulations were then conducted to predict and compare adalimumab concentration time profiles for two dosing regimens of interest: 40 mg eow vs. an 80-mg loading dose followed one week later by 40 mg eow.

### Results:

For patients in the Phase II trial, who were treated with adalimumab doses of 80 mg at Week 0 and 40 mg at Week 1, the mean adalimumab serum concentrations were 6.0 μg/mL and 8.8 μg/mL at Weeks 1 and 2, respectively. By comparison, the to steady-state concentrations during 40 mg eow dosing were 6.0 mg/mL and 6.9 μg/mL at Weeks 11 and 12. With the PK model, a total of 2,500 patients were simulated for each regimen (with and without an 80-mg loading dose one week before the start of a 40-mg eow dosing regimen). Clinical trial simulations demonstrated that a single 80-mg loading dose of adalimumab resulted in steady-state concentrations as early as Week 1, which was consistent with Phase II study results. The simulations also indicated that patients who do not receive loading doses may need approximately 12 weeks to reach therapeutic steady-state concentrations.

### Conclusions:

Clinical trial simulations demonstrated that a single 80-mg loading dose of adalimumab may help psoriasis patients achieve therapeutic steady-state drug concentrations substantially earlier (1 vs. 12 weeks) than without the loading dose.
References: ¹Gordon K B, et al. J Am Acad Dermatol. 2006; 55:598-606.

### Example 16

### Adalimumab is Efficacious in Patients With Moderate to Severe Psoriasis Regardless of Prior Exposure or Lack of Response to Systemic Therapies

### Aims:

Adalimumab is a fully human monoclonal antibody specific for tumor necrosis factor. This post-hoc subanalysis assessed response to adalimumab in patients with prior exposure to systemic therapies (biologics, non-biologics, and/or oral PUVA), and in patients who failed to respond to these therapies.

### Methods:

Data were pooled from three double-blind, placebo-controlled, efficacy and safety studies of adalimumab for the treatment of moderate to severe psoriasis: one 12-week, Phase II study of 147 patients (M02-528), and two 16-week, Phase III studies of 271 (CHAMPION) and 1,212 (REVEAL) patients. For the Phase III studies, study investigators collected patient histories of use of systemic psoriasis treatments over lifetime and response to systemic treatments administered within 12 months of study entry (patient recall of response to treatments administered more than 12 months before enrollment was not collected). For the Phase II study, patient histories of use of systemic treatments for psoriasis administered within 12 months of study entry and response to treatment within 12 months were collected. PASI response rates were assessed through Week 12 for the Phase II study and through Week 16 for the Phase III studies. Intention-to-treat analyses were conducted, with patients with missing PASI responses considered non-responders.

Results: The overall Week-4/Week-16 PASI 75 response rates were 19.4% (n=966)/72.1% (n=921) for adalimumab-treated patients, and 1.4% (n=503)/8.0% (n=451) for placebo-treated patients. PASI 75 response rates at Week 4/Week 16 for patients who had received systemic therapies were 21.7% (n=511)/72.7% (n=491) for adalimumab-treated patients, and 1.5% (n=269)/8.5% (n=247) for placebo-treated patients. PASI-75 response rates at Week 4/Week 16 for patients who had failed to respond to systemic therapy were 15.6% (n=160)/70.4% (n=152) for adalimumab-treated patients, and 0% (n=69)/8.1% (n=62) for placebo-treated patients. All results were statistically significantly greater for adalimumab vs. placebo (p<0.001). Additional results are provided in Tables 16-1 and 16-2, below.

TABLE 16-1
PASI 75 Response: Overall

| | Placebo | Adalimumab* |
|---|---|---|
| Week 4 | 1.4% | 19.4% |
| Week 8 | 3.8% | 54% |
| Week 12 | 5.8% | 68.1% |
| Week 16 | 8.0% | 72.1% |

*p < 0.001 adalimumab vs placebo.
ITT: patients with missing PASI scores considered nonresponders.

TABLE 16-2
PASI 75 Response: Week 16

| | Placebo | Adalimumab |
|---|---|---|
| Overall | 8.0% (N = 451) | 72.1%* (N = 921) |
| Lack of Response: MTX | 0.0% (N = 17) | 65.6%* (N = 32) |
| Lack of Response: | 20.0% (N = 5) | 71.4% (N = 7) |
| Cyclosporine | | |
| Lack of Response: | 50% (N = 4) | 83.3% (N = 6) |
| Oral PUVA | | |

*p < 0.001 adalimumab vs placebo.
ITT: patients with missing PASI scores considered nonresponders

Conclusions: Adalimumab is efficacious for the treatment of moderate-to-severe psoriasis. Adalimumab-treated patients who had received or had failed systemic therapies had similar responses compared with the overall population.

### Example 17

### Adalimumab for the Treatment of Fistulas in Patients with Crohn's Disease

### Overview:

This study was designed to evaluate the efficacy of adalimumab in the healing of draining fistulas in patients with active Crohn's disease (CD) as part of a Phase III, multicentre, randomised, double-blind, placebo-controlled study (CHARM). A subgroup of adults with moderate to severely active CD (CD Activity Index [CDAI] 220-450) who had draining fistulas at baseline were studied for ≧4 months. All patients received adalimumab induction therapy (80 mg at Week 0 and 40 mg at Week 2). At Week 4, all patients were randomised to receive placebo or adalimumab 40 mg every other week or weekly through Week 56 (irrespective of fistula status). Complete fistula healing (assessed at every visit) was defined as no drainage, either spontaneous or with gentle compression.

Of 854 patients enrolled, 778 were randomised at Week 4. At both screening and baseline visits, 117 patients had draining fistulas. Of these, 70 were randomized to receive adalimumab (combined 40 mg every other week or weekly) and 47 were randomized to receive placebo. At Week 26, complete fistula closure occurred in 21 of 70 patients (30%) in the combined adalimumab group and 6 of 47 patients (13%) in the placebo group (p=0.043). Of the patients in the combined adalimumab group, 100% of the 21 patients who had fistula closure at 26 weeks continued to have closure at 56 weeks, compared with 92% of the 6 patients in the placebo group. In patients with active CD, adalimumab therapy was more effective than placebo for inducing and then maintaining complete fistula healing.

### Methods:

### Study Design

Detailed CHARM study methodology was reported previously.⁶ This was a 56-week, multicentre, randomised, double-blind, placebo-controlled trial with a 4-week open-label induction period. At baseline, patients received open-label adalimumab 80 mg subcutaneously followed by 40 mg at Week 2. At Week 4, all patients still enrolled to were stratified by whether or not they achieved a clinical response (defined as achieving a decrease in Clinical Disease Activity Index [CDAI]≧70 points compared with baseline) and then randomized within each strata in a 1:1:1 ratio to one of three treatment groups, as follows: adalimumab 40 mg eow, adalimumab 40 mg weekly, or placebo.⁶

At or after Week 12, a patient with a flare or nonresponse could be switched to open-label treatment with 40 mg of adalimumab eow, which could be increased to 40 mg weekly if needed. Continued nonresponse on open-label adalimumab 40 mg weekly resulted in withdrawal from the study. A flare was defined as a recurrence of very active disease (CDAI increase ≧70 points from Week 4 and a CDAI>220). Nonresponse was defined as failure to achieve 70-point response at any visit at or after Week 12.

Patients classified as having fistulas were only those with draining fistulas at both screening and at baseline. Fistulas were assessed for spontaneous drainage or drainage with gentle compression at each study visit. Fistula efficacy endpoints included both complete fistula healing and at least 50% closure (ie, closure of at least 50% of draining fistulas that were present at baseline).

### Patients

Patients all had moderately to severely active CD, defined as a CDAI score between 220 and 450.¹² Enrollment with a history of infliximab treatment was permissible only if infliximab had been discontinued at least 12 weeks before the screening visit and the patient had experienced an initial response to the agent (as judged by the investigator). Demographic and baseline disease severity data, concomitant medication use at baseline, previous history of TNF antagonist use, and smoking history were collected for the entire population of patients in CHARM, but only patients with draining fistulas at screening and baseline are included in the subgroup analyses reported here. Baseline fistula data, that is number of fistulas and location, were noted and recorded.

### Efficacy Assessments

Complete fistula healing was defined as the absence of draining fistulas for at least the last two post-baseline evaluations on or before the study visit. Fistula response/improvement was defined as ≧□50% decrease from baseline in the number of draining fistulas for at least two consecutive visits. The following prespecified analyses were performed for the patients with fistulas: fistula closure by visit and determination of the mean number of draining fistulas per day of study. Fistula closure rates for patients with fistulas at baseline were also stratified by baseline immunosuppressant use (yes or no), baseline CD-related antibiotic use (yes or no), and previous history of use of any TNF antagonist (yes or no), as well as reason for discontinuation of the previous TNF antagonist (loss of response, adverse reaction, both). Assessments of clinical response and remission for the fistula cohort were also performed, including the percentage of patients with 70-point response (decrease in CDAI≧70), 100-point response (decrease in CDAI≧100), and clinical remission (CDAI<150)¹³ through Week 56. Also, the Inflammatory Bowel Disease Questionnaire (IBDQ)¹⁴ was used to assess health-related quality of life (HRQOL) based on changes from baseline in total IBDQ scores through Week 56 in the fistula cohort.

### Safety Assessments

To evaluate safety in the subset of patients with fistulas at baseline, serious adverse events and other adverse events of interest were collected throughout the study.

### Statistical Analysis

Fistula results were evaluated for all randomised patients with draining fistulas at both baseline and screening visits. The prespecified statistical analysis plan stipulated that fistula data from both adalimumab treatment groups (weekly and every other week) be combined (owing to small sample sizes and anticipated efficacy of adalimumab) and compared with the placebo group. All analyses were based on 2-sided tests with α=0.05. Continuous variables were compared using analyses of covariance adjusted for baseline values, and discrete variables were compared using Chi-square tests.

To calculate the draining fistulas per day, a new statistical approach was developed a priori to express the total fistula experience of each individual patient over the course of their participation in the study. Starting at Week 0 and ending at Week 56 (or study discontinuation), the number of draining fistulas at each pair of consecutive evaluations was averaged and multiplied by the elapsed days between the evaluations to obtain the number of draining fistula-days. The total number of draining fistula-days was summed from Week 0 through Week 56 (or study discontinuation) and divided by the total days in the study to obtain the average number of draining fistulas per day. The difference between each adalimumab group and the placebo group was assessed with least-squares means from the analysis of variance (ANOVA) with treatment as the only factor. Of note, the number of study days excluded days after discontinuation of double-blind study drug. Similarly, draining fistulas observed after study discontinuation or after discontinuation of double-blind treatment were excluded from the analysis. If a missing evaluation had non-missing double-blind evaluations before and after it, the average of the two non-missing evaluations was used to estimate the missing evaluation.

### Results:

### Patients

The demographics and patient disposition for the overall CHARM patient population were previously described.⁶ Of the 778 patients randomised at Week 4 to receive placebo (N=261), adalimumab 40 mg eow (N=260), or adalimumab 40 mg weekly (N=257), there was a total of 117 patients with draining fistulas at screening and baseline (placebo, n=47; 40 mg eow, n=30; and 40 mg weekly, n=40) (FIG. 2). Baseline characteristics are presented in table 17-1.

TABLE 17-1
Baseline demographics and clinical characteristics

| | | | Patients with fistulas at baseline | | | | | |
|---|---|---|---|---|---|---|---|---|
| | | | | | | | All patients | |
| | All patients | | Placebo | | Adalimumab | | with fistulas | |
| Characteristic | (N = 854) | | (n = 47) | | (n = 70) | | (N = 117) | |
| Male, n (%) | 326 | (38.2) | 15 | (31.9) | 34 | (48.6) | 49 | (41.9) |
| Age (y), mean (SD) | 37.1 | (11.9) | 36.5 | (10.1) | 35.9 | (12.2) | 36.1 | (11.4) |
| Baseline CDAI score, | 313.1 | (62.0) | 308.0 | (59.3) | 318.4 | (55.8) | 314.3 | (57.2) |
| mean (SD) | | | | | | | | |
| CRP (mg/dL) | | | | | | | | |
| Mean (SD) | 2.3 | (3.4) | 3.6 | (4.7) | 2.8 | (3.0) | 3.1 | (3.8) |
| Median (range) | 0.9 | (0.02-35.0) | 2.3 | (0.12-28.7) | 1.8 | (0.06-12.3) | 1.9 | (0.06-28.7) |
| CRP concentration | 407 | (47.7) | 32 | (68.1) | 42 | (60.0) | 74 | (63.2) |
| ≧1.0 mg/dL (10 mg/L), | | | | | | | | |
| n (%) | | | | | | | | |
| Previous TNF- | 424 | (49.6) | 31 | (66.0) | 41 | (58.6) | 72 | (61.5) |
| antagonist exposure, | | | | | | | | |
| n (%) | | | | | | | | |
| Concomitant | | | | | | | | |
| medication, n (%) | | | | | | | | |
| Any glucocorticoid* | 376 | (44.0) | 20 | (42.6) | 29 | (41.4) | 49 | (41.9) |
| Prednisone | 244 | (28.6) | 14 | (29.8) | 18 | (25.7) | 32 | (27.4) |
| Budesonide | 100 | (11.7) | 9 | (19.1) | 2 | (2.9) | 11 | (9.4) |
| Any | 399 | (46.7) | 26 | (55.3) | 31 | (44.3) | 57 | (48.7) |
| immunosuppressive | | | | | | | | |
| agent | | | | | | | | |
| Azathioprine | 275 | (32.2) | 21 | (44.7) | 24 | (34.3) | 45 | (38.5) |
| 6-Mercaptopurine | 81 | (9.5) | 4 | (8.5) | 3 | (4.3) | 7 | (6.0) |
| Methotrexate | 90 | (10.5) | 3 | (6.4) | 7 | (10.0) | 10 | (8.5) |
| 5-aminosalicylates^{†} | 334 | (39.1) | 13 | (27.7) | 24 | (34.3) | 37 | (31.6) |
| Current smoker, n | 303 | (35.5) | 18 | (38.3) | 20 | (28.6) | 38 | (32.5) |
| (%) | | | | | | | | |

CDAI, Crohn's Disease Activity Index;
CRP, C-reactive protein;
TNF, tumour necrosis factor.
*Includes betamethasone, budesonide, dexamethasone, deflazacort, cortisone, cloprednol, fluocortolone, glucocorticoids, hydrocortisone, methylprednisolone, prednisolone, prednisone, paramethasone, and prednylidene.
^{†}Aminosalicylic acid, balsalazide, mesalazine, olsalazine, sulfasalazine

Baseline characteristics of the patients with draining fistulas at screening and baseline were similar compared with the remainder of the patients, with the exception of numerically greater C-reactive protein concentrations and prior history of TNF-antagonist exposure in the patients with fistulising disease. Baseline fistula data are presented in Table 17-2.

TABLE 17-2
Baseline fistula data

| | | | Adalimumab |
|---|---|---|---|
| | | Adalimumab | 40 mg |
| | Placebo | 40 mg eow | weekly |
| Characteristic | (n = 47) | (n = 30) | (n = 40) |
| Draining cutaneous fistulas,* n (%) | | | |
| One | 30 (64) | 19 (63) | 23 (58) |
| Two | 7 (15) | 6 (20) | 7 (18) |
| Three | 6 (13) | 1 (3) | 5 (13) |
| Four | 4 (9) | 4 (13) | 5 (13) |
| Perianal fistulas,^{†} n (%) | | | |
| One | 29 (64) | 19 (63) | 21 (55) |
| Two | 7 (16) | 6 (20) | 7 (18) |
| Three | 6 (13) | 1 (3) | 5 (13) |
| Four | 3 (7) | 4 (13) | 5 (13) |

eow, every other week.
*Draining cutaneous fistulas includes perianal fistulas (n = 113) and abdominal fistulas (n = 4).
^{†}n = 45 for placebo, n = 30 for 40 mg adalimumab eow, n = 38 for 40 mg adalimumab weekly.

### Efficacy

Significantly greater percentages of patients receiving adalimumab treatment versus placebo had improvements of draining fistulas at Weeks 26 and 56 (Table 17-3). For patients with fistulas at baseline (n=117), patients who were treated with adalimumab (n=70) experienced a statistically significant and sustained increase in fistula closure over time compared with patients who received placebo (n=47): 30% or more of adalimumab patients experienced fistula closure, whereas less than 20% of the placebo patients experience fistula closure (week 12 through 56).

TABLE 17-3
Improvement in Draining Fistulas at Wk 26 and 56 (% of Patients)

| | Week 26 | Week 56 |
|---|---|---|
| Placebo (N = 47) | 28% | 26% |
| 40 mg EOW (N = 30) | 47% | 53% |
| 40 mg EW (N = 40) | 45% | 45% |
| Both ADA groups* (N = 70) | 46% | 49% |

During double-blind therapy in the randomised population of patients with draining fistulas at screening and baseline visits.
Note:
statistical analyses were not performed on individual adalimumab groups.
*p = 0.055 for combined adalimumab groups versus placebo at Week 26; p = 0.013 for combined adalimumab groups versus placebo at Week 56.
EOW, every other week;
EW, every week;
N, number of patients with fistulas at baseline

The new statistical methodology was used to calculate the number of draining fistulas per day during the double-blind period (Table 17-4). For all randomised patients, and also all randomised responder patients (those with Week-4 CDAI decreases ≧70 points versus baseline), there were significant decreases in the mean number of draining fistulas per day among adalimumab-treated patients compared with placebo-treated patients during the double-blind treatment period (randomised patients: mean=1.34 for placebo, mean=0.88 for adalimumab groups combined, p=0.002; randomised responder patients: mean=1.15 for placebo, mean=0.76 for adalimumab groups to combined, p=0.043).

TABLE 17-4
Mean Number of Draining Fistulas (Per Day, Double-Blind Period)

| | | Randomized |
|---|---|---|
| | All Randomized Patients^{†} | Responders^{‡} |
| Placebo (N = 47) | 1.34 | 1.15 |
| 40 mg EOW (N = 30) | 0.85 | 0.93 |
| 40 mg EW (N = 40) | 0.91 | 0.65 |
| Both ADA groups* (N = 70) | 0.88 | 0.76 |

*Statistical analyses were not performed on individual adalimumab groups, per prespecified statistical plan.
^{†}ITT population of patients with draining fistulas at screening and baseline visits.
^{‡}Randomised responder population (achieved 70-point response at Week 4) of patients with draining fistulas at screening and baseline visits.
eow, every other week;
ew, every week;
ITT, intention-to-treat;
N, number of patients with fistulas at baseline.

Baseline immunosuppressant or CD-related antibiotic use as well as previous exposure to TNF antagonists had no apparent effect on rates of fistula closure in patients receiving adalimumab or placebo at Weeks 26 or 56, although the numbers of patients in each group were small (Table 17-5, for all patients with fistulas at baseline (N=117) stratified by baseline concomitant therapies).

TABLE 17-5
Percentage of Patients with No Draining Fistulas at Weeks 26 and 56

| | No draining fistulas* | |
|---|---|---|
| | Week 26 | Week 56 |
| Subgroup | % Patients | % Patients |
| No baseline immunosuppressant use | | |
| Placebo (n = 21) | 19 | 19 |
| Both adalimumab groups (n = 39) | 33 (p = 0.369) | 36 (p = 0.241) |
| Baseline immunosuppressant use | | |
| Placebo (n = 26) | 8 | 8 |
| Both adalimumab groups (n = 31) | 26 (p = 0.092) | 29 (p = 0.051) |
| No baseline Crohn's disease-related | | |
| antibiotic use | | |
| Placebo (n = 28) | 14 | 14 |
| Both adalimumab groups (n = 44) | 32 (p = 0.162) | 36 (p = 0.059) |
| Baseline Crohn's disease-related | | |
| antibiotic use | | |
| Placebo (n = 19) | 11 | 11 |
| Both adalimumab groups (n = 26) | 27 (p = 0.264) | 27 (p = 0.264) |

*Patients who had no draining fistulas at the last two post-baseline evaluations in the double-blind period on or before the Week-26 or Week-56 visits were classified as no; otherwise, patients were classified as yes.

### Safety

Adalimumab was well-tolerated for up to 56 weeks of treatment in patients with fistulas at baseline (table 17-6), similar to the findings for all randomised patients reported by Colombel et al.⁶

TABLE 17-6
Summary of Safety in Cohort of Patients with Fistulas

| | Placebo | All adalimumab |
|---|---|---|
| | (n = 47) | (n = 70) |
| Event | n (%) | n (%) |
| Adverse event | 38 (80.9) | 59 (84.3) |
| Serious adverse event | 5 (10.6) | 9 (12.9) |
| Adverse event leading to discontinuation | 3 (6.4) | 4 (5.7) |
| of study medication | | |
| Infectious adverse event | 16 (34.0) | 31 (44.3) |
| Serious infectious adverse event* | 2 (4.3) | 5 (7.1) |
| Abscess (all) | 5 (10.6) | 8 (11.4) |
| Malignant neoplasm | 0 | 0 |
| Injection-site reaction (all) | 2 (4.3) | 3 (4.3) |
| Opportunistic infection^{†} | 1 (2.1) | 0 |
| Congestive heart failure | 0 | 0 |
| Demyelinating disorder | 0 | 0 |
| Death | 0 | 0 |

*Both of the placebo-treated patients had an abdominal abscess. The serious infectious adverse events for the 5 adalimumab-treated patients were a pulmonary embolus with pneumonia (n = 1); intra-abdominal abscess (n = 1); perianal abscess (n = 2); and scrotal abscess (n = 1).
^{†}Oral candidiasis.

### Discussion:

These data extend and define the beneficial effect of adalimumab on fistula healing in patients with CD presented in Example 3. This study also introduces and utilises a new methodology for evaluating the effect of treatment in patients with fistulising disease. The demographic characteristics of patients with fistulising disease did not differ substantially from the overall study population in CHARM. In this subgroup of patients, adalimumab demonstrated a consistent and significant reduction of draining fistulas when compared with placebo in patients with moderate to severe CD. Improvement in draining fistulas occurred in significantly more adalimumab-treated patients than placebo-treated patients at Weeks 26 and 56, consistent with previous findings regarding complete fistula closure.⁶ The effect of adalimumab on fistula closure was durable, as demonstrated by 100% of the adalimumab-treated patients who had fistula improvement at 26 weeks continuing to have closure at 56 weeks. In addition, two patients with draining fistulas at Week 26 had complete fistula healing at Week 56, suggesting that some patients may require long-term therapy to achieve complete healing.

Adalimumab therapy was associated with progressive increases in fistula closure over time, with separation in the rates of complete closure between placebo and adalimumab groups evident as early as 2 weeks after randomisation. Statistically significant differences in fistula closure between placebo and adalimumab were first observed at 12 weeks; this may have been appreciated earlier if a larger sample size had been available.

The efficacy of adalimumab was consistent in subgroups of patients who were receiving concomitant therapy with immunosuppressive agents or not, antibiotics or not, or who had a history of previous TNF-antagonist therapy use or not.

In addition to standard assessments of fistula closure at set time points, a new, prespecified statistical method was used to determine the number of draining fistulas per day. This new methodology may yield a more accurate picture of the total fistula experience of each individual patient over the full course of participation in the study by providing a longitudinal perspective. This analysis, which facilitates the comparison of subgroups of patients within an individual study, was prespecified in the statistical analysis plan for the CHARM study. A sample of the statistical analysis is depicted in Tables 17-7 and 17-8. Using the definition of complete fistula healing, hypothetical Patient 1 (Table 17-7) would have been assessed as not having successful closure of fistulas at either individual time point (Week 26 and Week 56), whereas hypothetical Patient 2 (Table 17-8) would have been assessed as having successful closure at Week 56. However, evaluation of “fistulas per day” demonstrates that hypothetical Patient 1 actually had a lower fistula burden over the study period than did hypothetical Patient 2, which more accurately reflects disease activity over the entire study period, instead of focusing on fistula counts at single points in time as does the definition of fistula healing. One limitation of this method is that between the visits, the numbers of draining fistulas on each day are assumed to remain the same and approximate the average number of draining fistulas observed at each visit. This assumption requires validation in appropriately constructed clinical trials.

TABLE 17-7
Hypothetical Patient 1: Minimal disease activity yet not achieveing
completing healing* at either Week 26 or Week 56

| | | Average | Patient |
|---|---|---|---|
| | | Fistula Count | Fistula Days |
| | Fistula Count | (AFC) | (PFD) |
| Baseline | 4 | | |
| | | 3.5 | 49 |
| Week 2 | 3 | | |
| | | 2 | 28 |
| Week 4 | 1 | | |
| | | 0.5 | 7 |
| Week 6 | 0 | | |
| | | 0 | 0 |
| Week 8 | 0 | | |
| | | 0 | 0 |
| Week 12 | 0 | | |
| | | 0.5 | 14 |
| Week 16 | 1 | | |
| | | 1 | 28 |
| Week 20 | 1 | | |
| | | 0.5 | 21 |
| Week 26 | 0 | | |
| | | 0 | 0 |
| Week 32 | 0 | | |
| | | 0 | 0 |
| Week 40 | 0 | | |
| | | 0.5 | 28 |
| Week 48 | 1 | | |
| | | 0.5 | 28 |
| Week 56/ | 0 | | |
| Early term | | | |

| | |
|---|---|
| Study Patient Fistula Days (SPFD) | 203 |
| Mean Number of Draining Fistulas | 0.52 |
| Per Day of Study | |

*Complete healing, no draining fistulas for at least their last 2 post-baseline evaluations in double-blind period.
** SPFD/Total Number of Days in Study = 203/392 days

TABLE 17-8
Hypothetical Patient 2: Significant disease activity yet achievement of
completing healing* at either Week 26 or Week 56

| | | Average | Patient |
|---|---|---|---|
| | | Fistula Count | Fistula Days |
| | Fistula Count | (AFC) | (PFD) |
| Baseline | 2 | | |
| | | 2 | 28 |
| Week 2 | 2 | | |
| | | 3 | 42 |
| Week 4 | 4 | | |
| | | 3.5 | 49 |
| Week 6 | 3 | | |
| | | 2.5 | 35 |
| Week 8 | 2 | | |
| | | 2.5 | 70 |
| Week 12 | 3 | | |
| | | 3 | 84 |
| Week 16 | 3 | | |
| | | 3 | 84 |
| Week 20 | 3 | | |
| | | 2 | 84 |
| Week 26 | 1 | | |
| | | 1.5 | 63 |
| Week 32 | 2 | | |
| | | 2 | 112 |
| Week 40 | 2 | | |
| | | 1 | 56 |
| Week 48 | 0 | | |
| | | 0 | 0 |
| Week 56/ | 0 | | |
| Early term | | | |

| | |
|---|---|
| Study Patient Fistula Days (SPFD) | 707 |
| Mean Number of Draining Fistulas | 1.80 |
| Per Day of Study** | |

*Complete healing, no draining fistulas for at least their last 2 post-baseline evaluations in double-blind period.
**SPFD/Total Number of Days in Study = 707/392 days

Infliximab, the only other TNF antagonist with marketing authorization for the treatment of CD throughout the European Union and the United States, has been shown to be effective in inducing and maintaining fistula closure.^{4,5} In the ACCENT II study (A to Crohn's Disease Clinical Study Evaluating Infliximab in a New Long-term Treatment Regimen II), 36% of patients who had responded to induction therapy and received maintenance infliximab therapy maintained complete fistula closure compared with 19% of patients receiving placebo (p=0.009).⁵ In ACCENT II, patients were included based on a minimal duration of fistula activity; only patients who responded to initial induction therapy were included in the analysis of maintenance efficacy.⁵ In CHARM, patients were included based on overall activity of their CD and not on fistula activity and all patients with fistulas were included in the analysis irrespective of initial response to adalimumab. Despite these differences, the fistula closure rates from ACCENT II at Week 54 are generally comparable to the fistula closure rates observed with adalimumab treatment at Week 56. In two studies of certolizumab pegol, a pegylated Fab′ fragment of an anti-TNF monoclonal antibody, the effect of certolizumab on fistula closure was not statistically significantly different compared with placebo.^{15,16}

Patients with fistulas at baseline tolerated sustained adalimumab treatment well. The safety profile of adalimumab in CHARM was consistent with studies of adalimumab in rheumatologic disorders and with previous studies of patients with CD. The safety of TNF antagonists in patients with fistulising disease is of concern, because of the greater risk of infection at baseline (especially abscesses) for these patients.¹⁷ There were no differences in the rates of adverse events, including infectious adverse events and, specifically, abscesses, in patients who received placebo versus those who received adalimumab at either dosage. This is consistent with previous reports.¹⁸

### Conclusions:

Significant and complete healing of draining fistulas was observed in adalimumab-treated patients, and healing of draining fistulas was maintained over time. Adalimumab maintenance therapy demonstrated a consistent, statistically significant benefit when compared with placebo. Adalimumab was well-tolerated with a safety profile consistent with previous studies in patients with CD.

### REFERENCES

1 Schwartz D A, Pemberton J H, Sandborn W J. Diagnosis and treatment of perianal fistulas in Crohn disease. Ann Intern Med 2001; 135:906-18.
2 Schwartz D A, Loftus E V Jr, Tremaine W J, et al. The natural history of fistulizing Crohn's disease in Olmsted County, Minnesota. Gastroenterology 2002; 122:875-80.
3 Lapidus A, Bernell O, Hellers G, et al. Clinical course of colorectal Crohn's disease: a 35-year follow-up study of 507 patients. Gastroenterology 1998; 114:1151-60.
4 Present D H, Rutgeerts P, Targan S, et al. Infliximab for the treatment of fistulas in patients with Crohn's disease. N Engl J Med 1999; 340:1398-405.
5 Sands B E, Anderson F H, Bernstein C N, et al. Infliximab maintenance therapy for fistulizing Crohn's disease. N Engl J Med 2004; 350:876-85.
6 Colombel J F, Sandborn W J, Rutgeerts P, et al. Adalimumab for Maintenance of Clinical Response and Remission in Patients With Crohn's Disease: the CHARM trial. Gastroenterology 2007; 132:52-65.
7 Sandborn W J, Hanauer S B, Rutgeerts P, et al. Adalimumab for maintenance treatment of Crohn's disease: results of the CLASSIC II trial. Gut 2007; 56:1232-9.
8 Hinojosa J, Gomollon F, Garcia S, et al. Spanish Scientific Group on Crohn's Disease and Ulcerative Colitis. Efficacy and safety of short-term adalimumab to treatment in patients with active Crohn's disease who lost response or showed intolerance to infliximab: a prospective, open-label, multicentre trial. Aliment Pharmacol Ther 2007; 25:409-18.
9 Hanauer S B, Sandborn W J, Rutgeerts P, et al. Human anti-tumor necrosis factor monoclonal antibody (adalimumab) in Crohn's disease: the CLASSIC-I trial. Gastroenterology 2006; 130:323-33.
10 Sandborn W J, Hanauer S, Loftus E V Jr, et al. An open-label study of the human anti-TNF monoclonal antibody adalimumab in subjects with prior loss of response or intolerance to infliximab for Crohn's disease. Am J Gastroenterol 2004; 99:1984-9.
11 Sandborn W J, Rutgeerts P, Enns R, et al. Adalimumab induction therapy for Crohn's disease previously treated with infliximab: a randomized trial. Ann Intern Med 2007; 146:829-38.
12 Best W, Becktel J, Singleton J, et al. Development of a Crohn's Disease Activity Index: National Cooperative Crohn's Disease Study. Gastroenterology 1976; 70:439-44.
13 Sandborn W J, Feagan B G, Hanauer S B, et al. A review of activity indices and efficacy endpoints for clinical trials of medical therapy in adults with Crohn's disease. Gastroenterology 2002; 122:512-30.
14 Irvine E J, Feagan B, Rochon J, et al. Quality of life: a valid and reliable measure of therapeutic efficacy in the treatment of inflammatory bowel disease. Canadian Crohn's Relapse Prevention Trial Study Group. Gastroenterology 1994; 106:287-96.
15 Sandborn W J, Feagan B G, Stoinov S, et al; PRECISE 1 Study Investigators. Certolizumab pegol for the treatment of Crohn's disease. N Engl J Med 2007; 357:228-38.
16 Schreiber S, Khaliq-Kareemi M, Lawrance I C, et al; PRECISE 2 Study

Investigators. Maintenance therapy with certolizumab pegol for Crohn's disease. N Engl J Med 2007; 357:239-50.
17 Present D H. Crohn's fistula: current concepts in management. Gastroenterology 2001; 124:1629-35.
18 Sands B E, Blank M A, Diamond R H, et al. Maintenance infliximab does not result in increased abscess development in fistulizing Crohn's disease: results from the ACCENT II study. Aliment Pharmacol Ther 2007; 23:1127-36.

### EQUIVALENTS

Those skilled in the art will recognize, or be able to ascertain using no more than routine experimentation, many equivalents to the specific embodiments of the invention described herein. Such equivalents are intended to be encompassed by the following claims. The contents of all references, patents, applications, and published patent applications cited throughout this application are incorporated herein by reference.

1. A method of determining the efficacy of a TNFα inhibitor for achieving a clinical response in Crohn's disease or maintaining remission of Crohn's disease in a subject comprising
determining the HRQL of a patient population having Crohn's disease and who were administered the TNFα inhibitor,
wherein a statistically significant improvement in the HRQL of the patient population indicates that the TNFα inhibitor is an effective for achieving a clinical response in Crohn's disease in a subject.

2. The method of claim 1, wherein determining the HRQL comprises using one or more Patient Related Outcome (PRO) scores or scales selected from the group consisting of IBDQ score, SF-36 PCS score, SF-36 MCS score, FACIT-fatigue score, Zung depression score, VAS score, and a combination thereof.

3. The method claim 1, wherein determining the HRQL comprises measuring the mean IBDQ score of the patient population, wherein a mean increase of 5 or more points in the IBDQ score of the patient population indicates that the TNFα inhibitor is effective for achieving a clinical response in Crohn's disease in a subject.

4. (canceled)

5. The method of claim 1, wherein determining the HRQL comprises measuring the SF-36 MCS score of the patient population, wherein an increase of 3 or more points in the SF-36 MCS of the patient population indicates that the TNFα inhibitor is effective for achieving a clinical response in Crohn's disease in a subject.

6. The method of claim 5, wherein a mean increase of 5 or more points in the SF-36 MCS of the patient population indicates that the TNFα inhibitor is effective for achieving a clinical response in Crohn's disease in a subject.

7. The method of claim 5, wherein a mean increase of 8 or more points in the SF-36 MCS of the patient population indicates that the TNFα inhibitor is effective for achieving a clinical response in Crohn's disease in a subject.

8-11. (canceled)

12. The method of claim 1, wherein determining the HRQL comprises measuring the mean FACIT-fatigue score, wherein a mean increase of 3 or more points in the FACIT-fatigue score of the patient population indicates that the TNFα inhibitor is effective for achieving a clinical response in Crohn's disease in a subject.

13. The method of claim 12, wherein a mean increase of 10 or more points in the FACIT-fatigue score of the patient population indicates that the TNFα inhibitor is effective for achieving a clinical response in Crohn's disease in a subject.

14. The method of claim 1, wherein determining the HRQL comprises measuring the mean Zung depression score of the patient population, wherein a mean decrease of 5 or more points in the Zung depression score of the patient population indicates that the TNFα inhibitor is effective for achieving a clinical response in Crohn's disease in a subject.

15. The method of claim 14, wherein a mean decrease of 9 or more points in the Zung depression score of the patient population indicates that the TNFα inhibitor is effective for achieving a clinical response in Crohn's disease in a subject.

16. The method of claim 1, wherein determining the HRQL comprises measuring the mean abdominal pain VAS score, wherein a mean increase of 4 or more points in the mean VAS score of the patient population indicates that the TNFα inhibitor is effective for achieving a clinical response in Crohn's disease in a subject.

17. The method of claim 1, wherein the determining the efficacy of the TNFα inhibitor further comprises determining the Crohn's Disease Activity Index (CDAI) score of the patient population, wherein a decrease of at least 70 in the CDAI score of at least 43% of the patient population indicates that the TNFα inhibitor is effective for achieving a clinical response in Crohn's disease in a subject.

18. The method of claim 1, further comprising administering the effective TNFα inhibitor to the subject to achieve a clinical response to Crohn's disease.

19. A method of achieving a clinical response in Crohn's disease in a subject comprising administering an effective TNFα inhibitor to the subject such that a clinical response in Crohn's disease is achieved, wherein the effective TNFα inhibitor was previously identified as causing a statistically significant improvement in a HRQL of a patient population having Crohn's disease as determined by a change in a PRO score selected from the group consisting of:
a) an increase 3 or more points in the SF-36 PCS score of a Crohn's Disease patient population;
b) an increase of about 3 or more points in the SF-36 MCS score of a Crohn's Disease patient population;
c) an increase of about 3 or more points in the FACIT-fatigue score of a Crohn's Disease patient population;
d) a decrease of about 5 points in the Zung depression score of a Crohn's Disease patient population;
e) a decrease of about 4 points in the Abdominal Pain VAS score of a Crohn's Disease patient population over 4 weeks of administration; and
f) a combination of two or more of (a)-(e).

20-37. (canceled)

38. A method of for maintaining remission of Crohn's disease in a subject. comprising administering an effective TNFα inhibitor to the subject such that a remission of in Crohn's disease is maintained, wherein the effective TNFα inhibitor was previously identified as causing a statistically significant improvement in a HRQL of a patient population having Crohn's disease as determined by a change in a PRO score selected from the group consisting of:
a) an increase 3 or more points in the SF-36 PCS score of a Crohn's Disease patient population;
b) an increase of about 3 or more points in the SF-36 MCS score of a Crohn's Disease patient population;
c) an increase of about 3 or more points in the FACIT-fatigue score of a Crohn's Disease patient population;
d) a decrease of about 5 points in the Zung depression score of a Crohn's Disease patient population;
e) a decrease of about 4 points in the Abdominal Pain VAS score of a Crohn's Disease patient population over 4 weeks of administration; and
f) a combination of two or more of (a)-(e).

39. The method of claim 1, wherein the TNFα inhibitor is a human TNFα antibody, or an antigen binding portion thereof, and wherein dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less.

40. The method of claim 39, wherein the human TNFα antibody, or an antigen-binding portion thereof, has the following characteristics:
a) dissociates from human TNFα with a K_{off} rate constant of 1×10⁻³ s⁻¹ or less, as determined by surface plasmon resonance;
b) has a light chain CDR3 domain comprising the amino acid sequence of SEQ ID NO: 3, or modified from SEQ ID NO: 3 by a single alanine substitution at position 1, 4, 5, 7 or 8 or by one to five conservative amino acid substitutions at positions 1, 3, 4, 6, 7, 8 and/or 9;
c) has a heavy chain CDR3 domain comprising the amino acid sequence of SEQ ID NO: 4, or modified from SEQ ID NO: 4 by a single alanine substitution at position 2, 3, 4, 5, 6, 8, 9, 10 or 11 or by one to five conservative amino acid substitutions at positions 2, 3, 4, 5, 6, 8, 9, 10, 11 and/or 12.

41. The method of claim 39, wherein the human TNFα antibody, or an antigen-binding portion thereof, comprises a light chain variable region (LCVR) having a CDR3 domain comprising the amino acid sequence of SEQ ID NO: 3, or modified from SEQ ID NO: 3 by a single alanine substitution at position 1, 4, 5, 7 or 8, and comprises a heavy chain variable region (HCVR) having a CDR3 domain comprising the amino acid sequence of SEQ ID NO: 4, or modified from SEQ ID NO: 4 by a single alanine substitution at position 2, 3, 4, 5, 6, 8, 9, 10 or 11.

42. The method of claim 39, wherein the human TNFα antibody, or an antigen-binding portion thereof, comprises a light chain variable region (LCVR) comprising the amino acid sequence of SEQ ID NO: 1 and a heavy chain variable region (HCVR) comprising the amino acid sequence of SEQ ID NO: 2.

43. The method of claim 39, wherein the human TNFα antibody, or an antigen-binding portion thereof, is adalimumab.

44-52. (canceled)


SEQUENCE LISTING (37 sequences)

SEQ ID NO: 1 - protein, 107 aa, Artificial [sequence omitted]

SEQ ID NO: 2 - protein, 121 aa, Artificial [sequence omitted]

SEQ ID NO: 3 - protein, 9 aa, Artificial
  D2E7 light chain variable region CDR3
QRYNRAPYX

SEQ ID NO: 4 - protein, 12 aa, Artificial
  D2E7 heavy chain variable region CDR3
VSYLSTASSLDX

SEQ ID NO: 5 - protein, 7 aa, Artificial
  D2E7 light chain variable region CDR2
AASTLQS

SEQ ID NO: 6 - protein, 17 aa, Artificial
  D2E7 heavy chain variable region CDR2
AITWNSGHIDYADSVEG

SEQ ID NO: 7 - protein, 11 aa, Artificial
  D2E7 light chain variable region CDR1
RASQGIRNYLA

SEQ ID NO: 8 - protein, 5 aa, Artificial
  D2E7 heavy chain variable region CDR1
DYAMH

SEQ ID NO: 9 - protein, 107 aa, Artificial
  2SD4 light chain variable region
DIQMTQSPSSLSASIGDRVTITCRASQGIRNYLAWYQQKPGKAPKLLIYAASTLQSGVPSRFSGSGSGTDFTLTISSLQPEDVATYYCQKYNSAPYAFGQGTKVEIK

SEQ ID NO: 10 - protein, 121 aa, Artificial
  2SD4 heavy chain variable region
QVQLVESGGGLVQPGRSLRLSCAASGFTFDDYAMHWVRQAPGKGLDWVSAITWNSGHIDYADSVEGRFAVSRDNAKNALYLQMNSLRPEDTAVYYCTKASYLSTSSSLDNWGQGTLVTVSS

SEQ ID NO: 11 - protein, 9 aa, Artificial
  2SD4 light chain variable region CDR3
QKYNSAPYA

SEQ ID NO: 12 - protein, 9 aa, Artificial
  EP B12  light chain variable region CDR3
QKYNRAPYA

SEQ ID NO: 13 - protein, 9 aa, Artificial
  VL10E4 light chain variable region CDR3
QKYQRAPYT

SEQ ID NO: 14 - protein, 9 aa, Artificial
  VL100A9 light chain variable region CDR3
QKYSSAPYT

SEQ ID NO: 15 - protein, 9 aa, Artificial
  VLL100D2 light chain variable region CDR3
QKYNSAPYT

SEQ ID NO: 16 - protein, 9 aa, Artificial
  VLL0F4 light chain variable region CDR3
QKYNRAPYT

SEQ ID NO: 17 - protein, 9 aa, Artificial
  LOE5 light chain variable region CDR3
QKYNSAPYY

SEQ ID NO: 18 - protein, 9 aa, Artificial
  VLLOG7 light chain variable region CDR3
QKYNSAPYN

SEQ ID NO: 19 - protein, 9 aa, Artificial
  VLLOG9 light chain variable region CDR3
QKYTSAPYT

SEQ ID NO: 20 - protein, 9 aa, Artificial
  VLLOH1 light chain variable region CDR3
QKYNRAPYN

SEQ ID NO: 21 - protein, 9 aa, Artificial
  VLLOH10 light chain variable region CDR3
QKYNSAAYS

SEQ ID NO: 22 - protein, 9 aa, Artificial
  VL1B7 light chain variable region CDR3
QQYNSAPDT

SEQ ID NO: 23 - protein, 9 aa, Artificial
  VL1C1 light chain variable region CDR3
QKYNSDPYT

SEQ ID NO: 24 - protein, 9 aa, Artificial
  VL0.1F4 light chain variable region CDR3
QKYISAPYT

SEQ ID NO: 25 - protein, 9 aa, Artificial
  VL0.1H8 light chain variable region CDR3
QKYNRPPYT

SEQ ID NO: 26 - protein, 9 aa, Artificial
  LOE7.A light chain variable region CDR3
QRYNRAPYA

SEQ ID NO: 27 - protein, 12 aa, Artificial
  2SD4 heavy chain variable region CDR3
ASYLSTSSSLDN

SEQ ID NO: 28 - protein, 12 aa, Artificial
  VH1B11 heavy chain variable region CDR3
ASYLSTSSSLDK

SEQ ID NO: 29 - protein, 12 aa, Artificial
  VH1D8 heavy chain variable region CDR3
ASYLSTSSSLDY

SEQ ID NO: 30 - protein, 12 aa, Artificial
  VH1A11 heavy chain variable region CDR3
ASYLSTSSSLDD

SEQ ID NO: 31 - protein, 12 aa, Artificial
  VH1B12 heavy chain variable region CDR3
ASYLSTSFSLDY

SEQ ID NO: 32 - protein, 12 aa, Artificial
  VH1E4 heavy chain variable region CDR3
ASYLSTSSSLHY

SEQ ID NO: 33 - protein, 12 aa, Artificial
  VH1F6 heavy chain variable region CDR3
ASFLSTSSSLEY

SEQ ID NO: 34 - protein, 12 aa, Artificial
  3C-H2 heavy chain variable region CDR3
ASYLSTASSLEY

SEQ ID NO: 35 - protein, 12 aa, Artificial
  VH1-D2.N heavy chain variable region CDR3
VSYLSTASSLDN

SEQ ID NO: 36 - DNA, 321 bp, Artificial
  D2E7 light chain variable region
gacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaa

SEQ ID NO: 37 - DNA, 363 bp, Artificial
  D2E7 heavy chain variable region
gaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagt